(19) United States
(12) Patent Application Publication
(10) Pub. No.: US 2005/0233963 A1
(43) Pub. Date: Oct. 20, 2005

(54) Heat shock response and virus replication

(75) Inventors: Pope Moseley, Albuquerque, NM (US); Matthew Cotten, Munich (DE); Brian Hjelle, Albuquerque, NM (US); Antonito Panganiban, Albuquerque, NM (US); Peter C. Angeletti, Lincoln, NE (US)

Correspondence Address: Henry D. Coleman;Coleman Sudol Sapone, P.C., 714 Colorado Avenue, Bridgeport, CT, 06605-1601, US

(73) Assignees: Science & Technology Corporation @ UNM; Wisconsin Alumni Research Foundation; Boehringer Ingelheim International

(21) Appl. No.: 11/047,063

(22) Filed: Jan. 31, 2005

Related U.S. Application Data
(62) Division of application No. 09/947,806, filed on Sep. 6, 2001.
(60) Provisional application No. 60/230,649, filed on Sep. 7, 2000.

(51) Int. Cl. A61K 38/17; A61K 48/00

(52) U.S. Cl. 514/12; 514/44

(57) ABSTRACT

The present invention discloses a method of inhibiting heat shock protein-dependent virus replication in cells and in animals. The present invention also discloses a method of identifying compounds which inhibit heat shock protein-dependent virus replication.

### CROSS REFERENCE TO RELATED APPLICATIONS

The present application claims priority under 35 U.S.C. § 119(e) to U.S. Provisional Application No. 60/230,649 filed on Sep. 7, 2000.

### STATEMENT REGARDING FEDERALLY SPONSORED RESEARCH AND DEVELOPMENT

The invention was made in part using funds obtained from the U.S. Government (National Institutes of Health Grant Nos. AR40771, AG14687, HL61389, AI41692 and GM63478). The U.S. Government may have certain rights in this invention.

### BACKGROUND OF THE INVENTION

Infection of an organism by a virus can produce effects ranging from minor symptoms to death. For successful viral infection to occur, viruses must redirect host biochemistry to replicate the viral genome an&produce and assemble progeny virions. Cellular heat shock responses, which are characterized as elevation and relocalization of heat shock proteins, occur during replication of many viruses (Nevins, 1982, Cell 29:913-919; Kan and Nevins, 1983, Mol. Cell. Biol. 3:2058-2065; Phillips et al., 1991, J. Virol. 65:5680-5692; Santomenna et al., 1990, J. Virol. 64:2033-2040; Ohgitani et al., 1999, J. Gen. Virol. 80:63-68; Kudayashi et al., 1994, Microbiol. Immunol. 36:321-325; Collins et al., 1982, J. Virol. 44:703-707). Viruses known to induce a heat shock response include herpes simplex, cytomegalovirus and adenovirus. The consensus has been that cells produce heat shock proteins early in a viral infection as a protective mechanism against the virus. Heat shock responses, which are stress responses by a cell, can occur following various types of perturbation. Such responses might be the reaction of the host to the synthesis of a foreign protein, or might be the consequence of the need of the virus to activate transcription. Alternatively, because heat shock proteins (HSPs) can facilitate protein folding, transport, and assembly (Mayer et al., 1998, Biol. Chem. 379:261-268; Kelley, 1999, Cun. Biol. 9:R305-308), the activation of a heat shock response by a virus might be a specific virus function which ensures proper synthesis of viral proteins and virions.

It has not been possible to determine whether a heat shock response is essential for virus replication, because the viral genes implicated in induction of such a response to date (for example, the adenovirus Ad5 E1 A gene) (Hint and Shenk, 1997, Ann. Rev. Genet. 31:177-212) also control other essential virus replication steps. Thus, inhibition of these genes by whatever means, results in inhibition of virus replication.

A heat shock type response associated with virus infection is not restricted to animal cells. DnaK and DnaJ, the bacterial heat shock protein 70 (hsp70) and heat shock protein 40 (hsp40) homologues, were originally identified as host factors essential for bacteriophage lambda replication (reviewed in Polissi et al., 1995, FEMS Microbiol. Rev. 17:159-169).

Specific heat shock proteins have been shown to be involved in virus replication. For example, hsp40 and hsp70 stimulate papillomavirus replication (Liu et al., 1998, J. Biol. Chem. 273:30704-30712) and DNA tumor virus T antigens have been shown to possess DnaJ-like activity that is important for remodeling nuclear protein complexes required for virus replication (Kelley, 1999, Curr. Biol. 9:R305-308; Campbell et al., 1997, Genes Dev. 11:1098-1100; Kelley and Georgopoulos, 1997, Proc. Natl. Acad. Sci. USA 94:3679-3684; Stubdal et al., 1997, Mol. Cell. Biol. 17:4979-4990; Srinivasan et al., 1997, Mol. Cell. Biol. 17:4761-4773). In addition, the viral R protein of HIV-1 has hsp70-like activity (Agostini et al., 2000, Exp. Cell Res. 259:398-403).

Members of the hsp70 family of heat shock proteins are the most highly responsive to heat. Hsp70 binds to peptides under conditions of high cellular ADP as is seen with states of energy depletion, including states such as torpor (Palleros et al., 1994, J. Biol. Chem. 269:13107-13114). ADP and ATP compete for a single site on hsp70. In addition to ADP/ATP, the hsp70 peptide complex includes the regulatory co-chaperone hsp40, which enhances both peptide binding and folding by the complex. Regulatory co-chaperones that associate with hsp70 often exert their effect by positively or negatively affecting the ATPase activity of hsp70. Hsp70 contains an intrinsic ATPase domain in the N-terminal segment, and cycles between an ATP-bound form and an ADP-bound form. ATP-bound hsp70 has relatively low affinity for substrate peptides, whereas the ADP-bound form has higher affinity and promotes more efficient protein folding. When a protein substrate occupies the substrate binding site of ADP-bound hsp70, a conformational change in the C-terminus takes place that results in tight association between hsp70 and the substrate. The ATP-bound hsp70 does not undergo this conformational change, and this accounts for the difference between high and low affinity substrate binding of hsp70. Hsp70 can also transfer substrate to hsp90, which can actively refold proteins.

Heat shock protein function in some cases appears to require protein-protein interactions. For example, hsp40 is recruited to hsp70 via interactions with the J domain of hsp40. Presentation of the J domain in the absence of other functional regions of hsp40 would be expected to block binding of hsp40 to hsp70 and to impair function. Indeed, the presence of a truncated hsp40 molecule comprising predominantly the J domain will impair hsp70/hsp40 induced refolding (Michels et al., 1999, J. Biol. Chem. 274:51:36757-36763).

Recent studies have suggested additional roles for viruses in interacting with a host cell's biochemical pathways. For example, gallus anti mort 1 (Gam1) protein, encoded by the avian adenovirus chicken embryo lethal orphan (CELO), was identified in an anti-apoptosis screen and was found to localize to the nucleus (Chiocca et al., 1997, J. Virol. 71:3168-3177). The nuclear location of Gam1 suggested that the protein might influence the expression of genes whose products could modulate apoptosis. Among these, hsp70 expression has been correlated with increased cell survival under stress (Gahai et al., 1997, J. Biol. Chem. 272:18033-18037; Mosser et al., 1997, Mol. Cell, Biol. 17:5317-5327). However, previous studies have not adequately addressed methods of inhibiting virus replication based on disrupting their reliance on cellular biochemical pathways.

There is a long felt need in the art for the development of new methods of inhibiting virus replication and for new antiviral compounds, especially compounds that target cellular functions essential for virus replication. The present invention satisfies these needs.

### SUMMARY OF THE INVENTION

The invention relates to a method of inhibiting virus replication in a cell wherein a heat shock protein is required for replication of the virus. The method comprises administering to a cell a virus replication-inhibiting amount of a heat shock protein inhibitor.

In one embodiment, the cell is an avian cell. In one aspect, the cell is a mammalian cell. In another aspect, the mammalian cell is a human cell.

In another embodiment of this aspect of the invention, the heat shock protein may be selected from the group consisting of a heat shock protein 27, a heat shock protein 40, a heat shock protein 70, and a heat shock protein 90α. In another aspect the heat shock protein is heat shock protein 40.

In another aspect, the heat shock protein inhibitor inhibits a heat shock protein interaction required for virus replication. In yet another aspect the heat shock protein inhibitor inhibits interaction of heat shock protein 40 with heat shock protein 70.

In one embodiment of the invention, the heat shock protein inhibitor is a peptide comprising a heat shock protein 40 J domain comprising SEQ ID NO:1. In yet another aspect the said heat shock protein inhibitor is a synthetic peptide comprising a heat shock protein 40 J domain. The invention also includes a heat shock protein 40 J domain which comprises from about amino acid 1 to amino acid 70 of SEQ ID NO:1.

The invention also relates to a method of inhibiting heat shock protein dependent virus replication comprising administering an isolated nucleic acid encoding a heat shock protein 40 J domain to a cell. When the nucleic acid is expressed in the cell the heat shock protein 40 J domain inhibits interaction of heat shock protein 40 with heat shock protein 70.

The invention further relates to a method of inhibiting virus replication wherein the viruses are selected from the group consisting of papillomavirus, cytomegalovirus, measles virus, Newcastle's disease virus, respiratory syncitial virus, herpes simplex virus, human immunodeficiency Virus 1, hantavirus and adenovirus. In one aspect the adenovirus is chicken embryo lethal orphan (CELO) virus.

In one embodiment, the heat shock protein inhibitor is selected from the group consisting of an isolated nucleic acid, an expression vector, an antisense nucleic acid, a protein, a peptide, an antibody, a transcription inhibitor, a translation inhibitor, and an antiviral agent.

The invention additionally relates to a method of inhibiting virus replication in an animal wherein a heat shock protein is required for virus replication. The method comprises administering to an animal a virus replication-inhibiting amount of a heat shock protein inhibitor. In one aspect, the heat shock protein required for virus replication is selected from the group consisting of heat shock protein 27, heat shock protein 40, heat shock protein 70, and heat shock protein 90α. In yet another aspect, the heat shock protein is heat shock protein 40.

In another aspect of this embodiment of the invention, the heat shock protein inhibitor inhibits interaction of a heat shock protein 40 with a heat shock protein 70. In yet another aspect the heat shock protein inhibitor is a peptide comprising a heat shock protein 40 J domain.

The invention also relates to a method of inhibiting heat shock protein dependent virus replication wherein the heat shock protein 40 J domain comprises from about amino acid 1 to amino acid 70 of SEQ ID NO:1.

The invention further relates to a kit for inhibiting heat shock protein dependent virus replication in a cell. The kit comprises a heat shock protein inhibitor, an applicator, and an instructional material for the use thereof. In one embodiment of the kit, the heat shock protein inhibitor is selected from the group consisting of a peptide comprising a heat shock protein 40 J domain, a nucleic acid encoding a heat shock protein 40 J domain, a nucleic acid complementary with a nucleic acid encoding a heat shock protein 40 J domain wherein the nucleic acid is in an antisense orientation, and an antibody that specifically binds with a heat shock protein 40 wherein when the antibody binds with hsp40 binding of hsp40 with hsp70 is inhibited.

The invention relates to a kit for inhibiting virus replication in an animal infected with a virus. The kit comprises a heat shock protein inhibitor, an applicator, and instructional materials.

The invention further relates to a method of inhibiting heat shock protein dependent virus replication comprising administering a virus replication-inhibiting amount of a flavonoid to a cell. In one embodiment, the flavonoid is selected from the group consisting of naringenin, naringin, morin, catechin, kaempferol, myricetin, phloretin, phlorizdin, rutin, 3-methylquercetin, and quercetin. In one aspect, the flavonoid is quercetin.

In another aspect, the invention relates to a method of using flavonoids to inhibit a virus is selected from the group consisting of a papillomavirus, a cytomegalovirus, a measles virus, a Newcastle's disease virus, a respiratory syncitial virus, a herpes simplex virus, a human immunodeficiency virus 1, a hantavirus and an adenovirus. In another aspect the virus is hantavirus. In yet another aspect, the virus is Sin Nombre hantavirus.

The invention also relates to an isolated nucleic acid complementary to a nucleic acid encoding a heat shock protein, or a fragment thereof; the complementary nucleic acid being in an antisense orientation. The complementary nucleic acid may be in the form of a vector and the vector therefore comprises the isolated nucleic acid.

The invention further relates to a composition comprising the isolated nucleic acid and a pharmaceutically-acceptable carrier. In one aspect, the invention also relates to a non-human transgenic mammal comprising the isolated nucleic acid.

Also included in the invention is a method of inhibiting heat shock protein dependent virus replication in a cell comprising administering a virus replication-inhibiting amount of an isolated, antisense orientation nucleic acid complementary to a nucleic acid encoding a heat shock protein, or a fragment thereof. In one aspect, the inhibited heat shock protein is selected from the group consisting of heat shock protein 27, heat shock protein 40, heat shock protein 70, heat shock protein 72 and heat shock protein 90.

In addition, the invention relates to a method of treating a virus related disease in an animal wherein a heat shock protein is required for virus replication. The method comprises administering a virus replication-inhibiting amount of a composition comprising an inhibitor of heat shock protein dependent virus replication and a pharmaceutically-acceptable carrier. In one aspect, the inhibitor is a flavonid. In yet another aspect, the inhibitor is quercetin.

The invention also includes a method of inhibiting heat shock protein dependent virus replication wherein the inhibitor is an isolated nucleic acid complementary to a nucleic acid encoding a heat shock protein, or a fragment thereof, and the complementary nucleic acid is in an antisense orientation.

Also included is a method of inhibiting heat shock protein dependent virus replication of human immunodeficiency virus-1 (HIV-1). The method comprises administering a virus replication-inhibiting amount of a heat shock protein inhibitor to a cell. In one aspect of the invention, the heat shock protein inhibitor comprises viral particle u binding protein (UBP), or a derivative or fragment thereof. In another aspect, the heat shock protein inhibitor inhibits a heat shock protein interaction required for virus replication. In yet another aspect, the heat shock protein inhibitor inhibits a heat shock protein function selected from the group consisting of heat shock protein ATPase activity and heat shock protein folding function activity.

The invention also relates to a non-human transgenic mammal comprising an isolated nucleic acid encoding a viral particle u binding protein (UBP), or a derivative or fragment thereof.

The invention further relates to a non-human transgenic mammal comprising an isolated nucleic acid encoding an inhibitor of heat shock protein dependent virus replication.

Also included in the invention is a method of inhibiting heat shock protein dependent virus replication in a cell comprising administering a virus replication-inhibiting amount of an isolated nucleic acid encoding viral particle u binding protein (UBP) or derivatives or fragments thereof. When the nucleic acid is expressed in the cell, the UBP protein, derivatives or fragment thereof inhibit the heat shock protein. In one aspect, the heat shock protein is heat shock protein 70. In another aspect, the heat shock protein is heat shock protein 90.

In addition, the invention relates to a method of treating a virus related disease or disorder in an animal. The method comprises administering a virus replication-inhibiting amount of a composition comprising an isolated nucleic acid encoding viral particle u binding protein (UBP) or derivatives or fragments thereof. The composition further comprises a pharmaceutically-acceptable carrier.

The invention further relates to a method of identifying a compound which inhibits heat shock protein dependent virus replication. The method comprises contacting a cell with a test compound, then comparing the level of heat shock protein function in that cell with the level of heat shock protein function in an otherwise identical cell not contacted with the test compound, wherein a lower level of heat shock protein function in the cell contacted with test compound compared with the level of heat shock protein function in an otherwise identical cell not contacted with test compound is an indication that the test compound inhibits heat shock protein function. When the test compound inhibits heat shock protein function, it is then added to a virus-infected cell. Then the level of virus replication in the cell is compared with the level of virus replication in an otherwise identical cell not contacted with test compound. A lower level of virus replication in the virus-infected cell contacted with test compound compared with the level of virus replication in an otherwise identical cell not contacted with test compound is an indication that the test compound inhibits virus replication.

In one aspect of this embodiment, the compound inhibits a heat shock protein selected from the group consisting heat shock protein 27, heat shock protein 40, heat shock protein 70, and heat shock protein 90. In another aspect, the compound inhibits heat shock protein 40. In yet another aspect, the compound inhibits heat shock protein 70. And in yet another aspect, the compound inhibits heat shock protein 90.

In one embodiment, the compound inhibits heat shock protein dependent virus replication of a virus selected from the group consisting of a papillomavirus, a cytomegalovirus, a measles virus, a Newcastle's disease virus, a respiratory syncitial virus, a herpes simplex virus, a human immunodeficiency virus 1, a hantavirus and an adenovirus. In one aspect the virus is selected from the group consisting of adenovirus, hantavirus, and human immunodeficiency virus-1.

In another aspect, the inhibited heat shock protein function is a heat shock protein interaction.

In yet another aspect, the inhibited heat shock protein function is ATPase activity. In yet another aspect, the inhibited heat shock protein function is heat shock protein folding activity.

In one aspect, the host cell is an avian cell. In yet another aspect, the host cell is a mammalian cell. In one aspect, the mammalian cell is a human cell.

In another aspect, the invention relates to a method of identifying an inhibitor of heat shock protein dependent virus replication wherein the heat shock protein function is a heat shock protein interaction. The method comprises contacting a cell with a test compound and comparing the level of interaction of a first heat shock protein with a second heat shock protein in the cell with the level of interaction of the first heat shock protein with the second heat shock protein in an otherwise identical cell not contacted with the test compound. A lower level of interaction of the first heat shock protein with the second heat shock protein in the cell contacted with the test compound compared with the level of interaction of the first heat shock protein with the second heat shock protein in an otherwise identical cell not contacted with test compound is an indication that the test compound inhibits a heat shock protein interaction. In one aspect of this embodiment, the first heat shock protein is selected from the group consisting of a heat shock protein 27, a heat shock protein 40, a heat shock protein 70, and a heat shock protein 90α. The invention also includes a compound identified by this method.

### BRIEF DESCRIPTION OF THE SEVERAL VIEWS OF THE DRAWINGS

The foregoing summary, as well as the following detailed description of preferred embodiments of the invention, will be better understood when read in conjunction with the appended drawings. For the purpose of illustrating the invention, there are shown in the drawings embodiments which are presently preferred. It should be understood, however, that the invention is not limited to the precise arrangements and instrumentalities shown. In the drawings:

FIG. 1, comprising FIG. 1a, FIG. 1b, and FIG. 1c, consists of images of SDS-polyacrylamide gels (left) and Western blots (right) depicting the effect of Gam1 expression on the levels of heat shock protein in cells.

FIG. 1a is an analysis of A549 cells infected with the indicated adenoviruses. Twenty-foul hours later, heat shock (43° C., 90 minutes) was applied as indicated. Forty-eight hours after infection, cell extracts were obtained and proteins obtained therefrom were resolved by SDS-PAGE and stained for total protein using Coomassie blue, or the proteins were assayed for the presence of hsp70 in a Western blot. Circles indicate viral transgene product. The conditions under which proteins were obtained for loading onto each lane are as follows: lanes 2-7, no heat shock; lanes 8-13, heat shock; non-infected cells (lanes 2 and 8); infection with Adβgal at 1,000 particles per cell (lanes 3 and 9) or 10,000 particles per cell (lanes 4 and 10); infection with AdGam1 at 100 particles per cell (lanes 5 and 11), 1,000 particles per cell (lanes 6 and 12), or 10,000 particles per cell (lanes 7 and 13).

FIG. 1b depicts an analysis of A549 cells infected with the indicated adenovirus at 1,000, 3,000 or 10,000 particles per cell. Protein extracts were prepared forty-eight hours after infection and were analyzed as described in FIG. 1A. The conditions under which proteins were obtained for loading onto each lane are as follows: lanes 2 and 12: non-infected cells; lanes 3-5, infection with AdGam1; lanes 6-8, infection with AdLuc; lanes 9-11, infection with AdEGFP.

FIG. 1c comprises Western blot analyses of Gam1 effects on heat shock proteins. A549 cells were infected with AdGam1 or AdLuc at 1,000, 3,000 or 10,000 particles per cell with extracts prepared 48 hours post-infection and immunoblotted for hsp70, hsp40, hsp27, hsc70, hsp90 or tubulin.

FIG. 2, comprising FIGS. 2a-2l, is a series of photomicrographs depicting the fact that hsp40 and hsp70 are upregulated by Gam1 expressed from a transfected plasmid. Immunofluorescence analysis of A549 cells which were not transfected (control, FIGS. 2a-2c, 2g-2i) or which were transfected with pGam1 (CMV immediate early promoter driving a Myc-Gam1 cDNA, FIGS. 2d-2f; 2j-2l) were stained for hsp40 (FIGS. 2a, 2d) or hsp70 (FIGS. 2g and 2j) and Myc for Myc-tagged Gam1 (FIGS. 2b, 2e, 2h, and 2k). Nuclei were counterstained with Hoechst dye (FIGS. 2c, 2f, 2i, 2l). In FIGS. 2f and 2l, arrows indicate cells with strong nuclear Gam1 expression and upregulation, and nuclear accumulation of hsp40 and hsp70, respectively.

FIG. 3, comprising FIGS. 3a-3i, is a series of photomicrographs illustrating that Gam1 induces relocation of hsp40 and hsp70 to the nucleus. Immunofluorescence analysis was performed on A549 cells infected with AdGam1 (FIGS. 3a-3c, 3g-3i) or AdGFP (FIGS. 3d-3f, 3j-3l) at 10,000 particles per cell. Cells were incubated with primary antibodies for anti-hsp40 (FIGS. 3a and 3d), anti-Myc for Myc-tagged Gam1 (FIGS. 3b and 3e), anti-hsp70 (FIGS. 3g, 3j) or GFP expression (FIGS. 3h and 3k), incubated with secondary antibodies, stained for expression, and then the nuclei were counterstained with Hoechst dye (FIGS. 3c, 3f, 3i, and 3l). In FIGS. 3c and 3l, arrows indicate cells with strong Gam1 expression and nuclear accumulation of hsp40 and hsp70; arrowheads indicate cells with weak (FIG. 3c) or no detectable Gam1 expression (FIG. 3i) and no nuclear accumulation of hsp40 or hsp70.

FIG. 4, comprising FIGS. 4a-4l, is a series of photomicrographs depicting that hsp40 and hsp70 are upregulated during CELO avian adenovirus infection. Immunofluorescence analysis was performed on LMH cells that were non-infected (FIGS. 4a-4c; 4g-4l) or infected with wild-type CELO (100 particles per cell: FIGS. 4d-4f; 4j-4l). Cells were labeled with anti-hsp40 (FIGS. 4a, 4d) or anti-hsp70 (FIGS. 4g, 4j) and with anti-CELO capsid (FIGS. 4b, 4e, 4h, 4k) antibodies. Nuclei were counterstained with Hoechst dye (FIGS. 4c, 4f, 4i, 4j).

FIG. 5, comprising FIGS. 5a-5e, depicts the fact that Gam1 is required for CELO replication and can be replaced by heat shock or hsp40.

FIG. 5a comprises two graphs wherein the left graph depicts replication of CELOdG alone and the right graph depicts replication of CELOdG plus AdGam1 at different passages. Luciferase activity (mean±s.d. of three cultures) is indicated for each passage.

FIG. 5b is an image of a Western blot.

FIG. 5c is a series of graphs depicting luciferase activity in LMH cells infected with 5 particles per cell of CELOwt or CELOdG.

Referring to both FIG. 5b and FIG. 5c, cells were incubated either without heat shock (FIG. 5b, lanes 1-3; FIG. 5c, lanes 1, 2), with a 90 minute, 45° C. heat shock twenty-four hours before infection (FIG. 5b, lanes 4-6; FIG. 5c, lanes 3 and 4), twenty-four hours after infection (FIG. 5b, lanes 7-9; FIG. 5c, lanes 5 and 6), or two hours before infection (FIG. 5b, lanes 10-12; FIG. 5c, lanes 7 and 8). After four days at 37° C., lysates were analyzed by immunoblotting for CELO capsid proteins (FIG. 5b) or for virus capable of transducing the luciferase gene into LMH cells (FIG. 5c).

FIG. 5d is a graph depicting luciferase activity in LMH cells (5×10⁵) infected with 1 virus particle per cell of CELOdG either alone or with 10,000 particles per cell of AdEGFP, AdGam1, Adhsp40 or Adhsp70. For each passage, lysates were prepared five days after infection, luciferase was measured (mean±s.d. of three results), and a fresh monolayer of LMH cells was infected (plus the indicated second virus).

FIG. 5e is an image of a Western blot depicting virus capsid proteins in LMH cells infected with 5 particles per cell of CELOwt, CELOdG or CELOdGhsp40. After five days at 37° C., equal quantities of lysate protein were analyzed by immunoblotting for virus capsid proteins. Lane 1, non-infected cells; lane 2, CELOwt; lane 3, CELOdG; lane 4, CELOdGhsp40.

FIG. 6, comprising FIGS. 6A, 6B, and 6C, illustrates Sin Nombre (SN) hantavirus (an RNA virus), reactivation by cold stress. Adult (10-14 weeks) outbred Peromyscus maniculatus N=5 per group) were inoculated in August 2000 (groups d60A and d90) or October 2000 (group d60B) and sacrificed 60 or 90 days later as indicated. The mice were maintained in an outdoor quarantine laboratory at ambient temperature. The graphs of FIG. 6A and FIG. 6B are aligned vertically to allow the temperature plot in FIG. 6A to be linked to specific experimental groups in FIG. 6B. FIG. 6A shows the ambient temperature at the laboratory in the 5 days immediately preceding the point at which the animals were euthanized. The ambient temperatures experienced by group 60A remained warm through the experimental period, never going below 0° C. The antigen load in this group remained low. A representative (negative) field from an immunohistochemical analysis (IHC) is shown in FIG. 6C (top micrograph, 400× in original). However, the ambient temperatures experienced in groups d60B and d90 were substantially lower, with overnight lows below freezing. In these latter groups, viral RNA load (FIG. 6B) was very high, at least equivalent to that seen in acute infection. The induction of SN virus replication was most prominent in brown adipose tissue (BAT) but reactivation was also seen in heart, either as a primary event or possibly as a result of secondary spread of virus from BAT or other tissues. The arrowheads in FIG. 6C point to examples of N antigen expression in BAT from representative animals. Rabbit anti-SN-N antibody was used as the primary antibody, followed by biotinylated anti-rabbit IgG and then alkaline phosphatase-streptavidin and AEC substrate for color development.

FIG. 7 depicts images from Western blot analyses of heat shock protein hsp70 and hantavirus N antigen levels in brown adipose tissue of cold-stressed deer mice. Hsp70 levels and viral N antigen levels increased in BAT of infected deer mice after cold stress. These mice were inoculated as juveniles with 5 ID₅₀ of SN77734 and euthanized at 14, 28, 120, and 180 days. The upper western blot examines hsp70 levels, and the lower blot, SN N antigen (aMr, 55 kDa). Viral N antigen levels were high in acute infection but had declined by 120 days. However, temperatures declined markedly 4 days before the 180 day timepoint, and SN viral antigen levels increased as measured by immunohistochemistry. While it appears that hsp70 and N antigen levels moved in lockstep in these studies of single animals, it is possible that HSP levels peaked before virus replication was stimulated and N antigen began to increase.

FIG. 8, comprising FIGS. 8A and 8B, demonstrates by western blot and immunohistochemical analyses that heat shock protein hsp72 expression can be experimentally induced in deer mice. FIG. 8A, depicts an image of a western blot showing hsp72 reactivity with Stressgen SPA 812 polyclonal antibody in liver: lane 1, untreated deer mouse; lane 2, mouse treated with phenylephrine IP (25 μg/kg) 6 hours before sacrifice; lane 3, mouse subjected to stress by placement in metabolic cage, removed 6 hours before sacrifice. Similar results were obtained with BAT. FIG. 8B comprises immunohistochemical analyses of hsp70 expression in adrenal glands (top panels) and BAT (bottom panels), comparing an unstressed mouse (left) with a mouse subjected to stress with phenylephrine (25 μg/kg) 6 hours before sacrifice. Note several-fold darker DAB stain in stressed adrenal and BAT. Original magnification was 200× (adrenals) and 400× (BAT).

FIG. 9 is an image of a western blot demonstrating that persistent hantavirus infection of Vero E6 cells induces hsc70, the constitutively expressed form of the hsp70 family. Uninfected cells (left lane) were compared with two independent isolates of SN77734 (middle lanes) and the California isolate of SN virus CC107 (far right lane) in this western blot. A monoclonal antibody specific for hsp70, i.e., does not bind to the constitutively expressed hsc70, failed to react with the middle band, which suggests that the induced form of the hsp70 family in Vero E6 cells is hsc70. However, in a separate study, it was found that an inducible form of the hsp70 family, hsp72 itself, was induced in infected or uninfected Vero E6 cells from the same very low basal level by heat shock. The observation that heat induced hsp72 but the virus induced hsc70 would not be unexpected, because the normally constitutively expressed hsc70 has been shown to be induced or associated with virus in preference to hsp70 in several types of viral infections (Sainis et al., 1994, FEBS Lett. 355:282-286; Saphire et al., 2000, J. Biol. Chem. 275:4298-4304).

FIG. 10, comprising FIGS. 10A, 10B, and 10C, demonstrates that heat shock (“ΔH”) reactivates SN virus in persistently infected cells. Confluent T25 flasks of persistently SN virus-infected and -uninfected Vero E6 cells were placed in a 43° water bath for 1.5 hours. FIG. 10A is an image of a western blot demonstrating hsp70 expression following heat shock. After the specified intervals, the cells were trypsinized and counted, and subjected to lysis with a Beadbeater in standard SDS-βME protein lysis buffer. Proteins from the equivalent of 2×10⁴ cells/lane were subjected to SDS-PAGE (12.5%) and transferred to nitrocellulose. The membrane was probed with Stressgen 812 hsp72-specific rabbit antibody. FIG. 10B is a graph depicting the production of SN77734 viral RNA in the clarified supernatant of Vero E6 cells that were subjected, or not subjected, to 1.5 hours of 43° heat shock. Five separate T25 flasks of cells were used, so that the cells could be trypsinized and subjected to study (FIG. 10A); for that reason, the viabilities were known at each time point, and in each case the viability was 100%. Peak induction was noted 96 hours after shock, the last timepoint studied. FIG. 10C is an image of a western blot analysis which demonstrates release of virions (SN virus N antigen) into supernatant. Each lane contains the equivalent of 12 μl of the same supernatant as in FIG. 10B, and is probed for N antigen by rabbit anti-SN N (1:5000). Purified recombinant N antigen (40 ng) was loaded in the left lane as a control. Note induction of N antigen at 72-96 hours with heat shock (+HS, lower panel) compared to the control (no HS; upper panel). By comparison, there was no increase in hsp70 released into medium.

FIG. 11 is a micrograph which demonstrates that infectious SN virus (hantavirus) can be quantitated by focus assay using antibody to N antigen. In this experiment infected Vero E6 cells were subjected to immunochemistry utilizing an antibody to N antigen, followed by horseradish peroxidase and DAB staining procedures. Stained SN virus foci are evidenced by dark brown clusters. The original magnification was 100×.

FIG. 12 demonstrates graphically that the flavonoid quercetin, an inhibitor of HSP induction, inhibits heat shock induced reactivation of SN hantavirus. Infected Vero E6 cells were subjected to heat shock (43°, 1.5 hours) with or without quercetin at 100 μM. SN viral RNA released into the medium was measured every 2 days for 10 days. It can be seen that heat shock induced high levels of viral RNA titers by day 10, but that cells heat shocked and treated with quercetin did not have increased levels of viral RNA titers. In fact, the titers in the heat shocked cells treated with quercetin were similar to those cells that were not subjected to heat shock. There was a statistically significant 7.3 fold inhibition (p=0.0003) of viral RNA titers in quercetin treated cells, compared to the statistically significant (p=0.013) 4.7 fold difference between cells subjected to heat and those not subjected to heat shock. Temporal changes in induction of viral RNA compared to other experiments may be due to slight differences in experimental conditions such as differences between freshly infected cells and persistently infected cells.

FIG. 13, comprising FIG. 13A, FIG. 13B, and FIG. 13C, illustrates by western and far-western analyses that UBP binds directly to hsp70 and hsc70 with high affinity. FIG. 13A: Thirty μg of HeLa cell lysate was separated by SDS-PAGE in three identical lanes, blotted to nitrocellulose and probed with either, no probe (none), GST alone, or GST-UBP. The far-western blot was then developed with anti-GST antibody to reveal bands which interact with UBP. The arrow to the right indicates a 70 kD band detected by the GST-UBP fusion. The numbers to the left of each blot represent molecular weights in kilodaltons. FIG. 13B: Identical blots containing 30 μg of HeLa lysate, 50 ng pure hsc70 and 50 ng pure hsp70, as indicated, were subject subjected to UBP Far-Western (UBP FW) or anti-hsp70 western analysis (αhsp70). The asterisk indicates the position of hsp90, also detected in HeLa cell lysate by UBP Far-Western interaction. FIG. 13C, Samples from the GST-UBP pull down assay were analyzed by western blot for the presence hsc70. At the bottom of the gel, the contents of each reaction are indicated by the plus symbols. The NaCl concentration of the washing buffer (100, 200, 300, and 500 mM) is indicated at the top of each lane. The arrow to the right of the blot indicates the position of the hsc70 band.

FIG. 14 demonstrates by far-western blot analysis and comparison to each fusion construct, the mapping of the UBP/hsc70 interaction. The term “hsc70” is used when referring to both hsp70 and its nearly identical constitutive counterpart, hsc70. Seven identical sets of blots containing 20 ng hsc70 and hsp70 were subjected to UBP far-western analysis. The blots were probed with GST fusions as indicated; full-length UBP, Δ1-93, Δ95-195, Δ288-313, N1/2 (a.a. 1-145), C1/2 (a.a. 145-313), and TPR2-4 (a.a. 95-195). The numbers to the left of the blots indicate the molecular weights in kilodaltons. The diagram below the blots indicates regions of UBP contained in each fusion construct. The column to the right summarizes the results, where a plus symbol indicates binding and a minus symbol indicates no binding.

FIG. 15, comprising FIG. 15A and FIG. 15B, demonstrates graphically that UBP inhibits hsc70-dependent ATPase activity. FIG. 15A: Twenty-eight nM hsc70 was incubated with 0, 12, 58, 118, or 176 nM UBP or BSA and 1 μCi of [α-³²P]ATP (13 nM) for 1 hr at 30° C. ADP was separated from ATP by thin-layer chromatography and quantified using a phosphorimager. Data are represented as the percent total ATPase activity where the hsc70 ATPase activity in absence of BSA or UBP is set to 100%. The hsc70 ATPase activity in the presence of BSA (diamonds) or UBP (squares) are graphed as shown. FIG. 15B: Twenty-eight nM hsc70 was incubated with 1 μCi of [α-³²P]ATP with or without UBP at a final concentration of 28 nM. Hydrolysis of ATP was monitored in the presence of hsc70 (triangle), hsc70 and UBP (circle), UBP alone (squares), or with no protein (diamond). The data are represented as the percent ATP hydrolysis and are plotted as a function of time from 0-75 minutes.

FIG. 16 demonstrates graphically that UBP inhibits hsc70-dependent refolding of denatured firefly luciferase. Heat-denatured luciferase was incubated with hsc70 (square), hsc70 and UBP (triangle), or no hsc70 (circle). Luciferase activity was quantified using a luminometer. Data are plotted as the relative light units (RLU) as a function of time from 0-100 minutes.

FIG. 17 demonstrates by far-western blot analysis and comparison to each fusion construct the mapping of the UBP/hsp90 interaction. Identical blots containing 20 ng hsp90 were developed by UBP far-western. The blots were probed with GST or GST fusions having: full-length UBP, Δ1-93, Δ95-195, Δ288-313, N1/2 (a.a. 1-145), C1/2 (a.a. 145-313), and TPR2-4 (a.a. 95-195). The numbers to the left of the blots indicate the molecular weights in kilodaltons. The regions of UBP contained in each fusion construct are diagrammed below the blots. The right column summarizes the results: a plus symbol indicates binding and a minus symbol indicates no binding.

FIG. 18, comprising FIG. 18A and FIG. 18B, depicts graphically (FIG. 18B) and photographically (FIG. 18A) that deletion mutants of ubp in Saccharomyces cerevisiae are defective for recovery from severe heat shock. Wt or ubp deletion strains were subject to heat shock at 55° C. for 1 hour then plated at appropriate dilutions to rich media. The plates (upper panel) at the top of the bar graph show representative surviving colonies from wt or ubp-deletion strains respectively. Surviving colonies were counted and the data were expressed as the percent total survivor where wt UBP was set to 100%.

FIG. 19 depicts a graph demonstrating that Gag protein is found co-complexed with hsp70 and UBP in the presence or the absence of Vpu. Lysates from HeLa cells transfected with either Vpu⁺ or Vpu⁻ proviral genomes were incubated with protein-A sepharose beads coated with either no antibody (None), anti-UBP antibody (αUBP), or anti-hsp70 antibody (αhsp70). Immunoprecipitates were subjected to a quantitative p24 Gag assay. The quantity of Gag detected in each condition is expressed in relative p24 units.

FIG. 20, comprising FIG. 20A and FIG. 20B, graphically illustrates that expression of UBP effects HIV-1 particle release in the presence of Vpu. FIG. 20A: HeLa cells were co-transfected with 1 μg of Vpu⁺ or Vpu⁻ proviral genomes and 10 μg pHIV-TARluc (Luc), pHIV-UBP (UBP), pHIV-UBP-N (UBP-N). FIG. 20B: Alternatively, HeLa cells were co-transfected with 1 μg of Vpu⁺ or Vpu⁻ proviral genomes and 10 μg pHIV-TARluc (Luc) or pHIV-FTPR. Thirty-six hours after transfection the amount of p24 Gag in the media and cell pellets was quantified using an antigen capture ELISA. The relative particle release is given by the ratio of extracellular to intracellular p24. The data were normalized to the control (Luc). The error bars represent the error of the mean for three independent experiments.

FIG. 21 illustrates and compares the constructs of several TPR-containing co-chaperones.

### DETAILED DESCRIPTION OF THE INVENTION

The invention relates to a method of inhibiting heat shock protein dependent virus replication in cells. It has been discovered in the present invention that the prior art view that during the heat shock response cells produce heat shock proteins (HSPs) early in a viral infection to protect themselves is incorrect. Instead, the present invention demonstrates that virus replication is dependent upon HSPs.

As used herein, “heat shock response” refers to a stress response by a cell to various perturbations, including viruses and heat shock. The response includes, but is not limited to, elevation and relocalization of a variety of HSPs, as well as interactions of HSPs with each other. Heat shock response should be construed to include other stress responses, such as cold stress, that activate HSPs.

Heat Shock Protein Regulation of Adenovirus Replication

The invention disclosed herein illustrates, inter alia, the importance of HSPs in adenovirus replication. In the present disclosure, the data presented in Example 1 establish that the Gam1 protein of CELO adenovirus induces expression and translocation of both hsp40 and hsp70 in A549 cells. It is further established that Gam1 protein is required for CELO adenovirus replication. Furthermore, it is disclosed herein that a replication-deficient adenovirus (Gam1-deficient) can be complemented for growth in LMH cells merely by overexpressing heat shock protein 40 (hsp40) in the cells. This is the first demonstration that a replication-deficient virus can be made competent using a human gene, namely an HSP. However, hsp70 was not found to be able to replace or complement Gam1. Also disclosed is the fact that cellular HSP-HSP interactions are required for virus replication. This supports the notion that induction of a heat shock response is an essential viral function for virus replication and is not merely a cellular adaptive response to infection.

Heat Shock Protein Regulation of Hantavirus Replication

The present disclosure also illustrates by way of in vivo and in vitro studies in Example 2 that hantavirus infection and reactivation are both associated with cellular HSP responses. Specifically disclosed herein are data establishing that heat shock, as well as other stressors, regulate HSP expression and hantavirus replication in infected animals and in infected cells in culture. For example, it is illustrated herein that stress induces higher levels of hantavirus viral antigen expression in mice and activates HSPs in brown adipose tissue of mice. It is further illustrated that hantavirus can induce HSPs. Persistent infection of animal cells with hantavirus induces higher levels of expression of hsc70, a member of the hsp70 family which is normally constitutively expressed, to the extent that hsc70 is constitutively over-expressed. Furthermore, it has been discovered that a known inhibitor of cellular HSP transcription, the flavonoid quercetin, can inhibit heat shock induced reactivation of hantavirus in infected cells. The invention also discloses that results obtained in vivo regarding stress and virus induced heat shock proteins and stress induced virus activation can be modeled in vitro. Thus, the present disclosure establishes that inhibitors of heat shock protein pathways can potentially inhibit both infection of uninfected cells and reactivation of virus in infected cells.

Heat Shock Protein Regulation of HIV-1 Replication

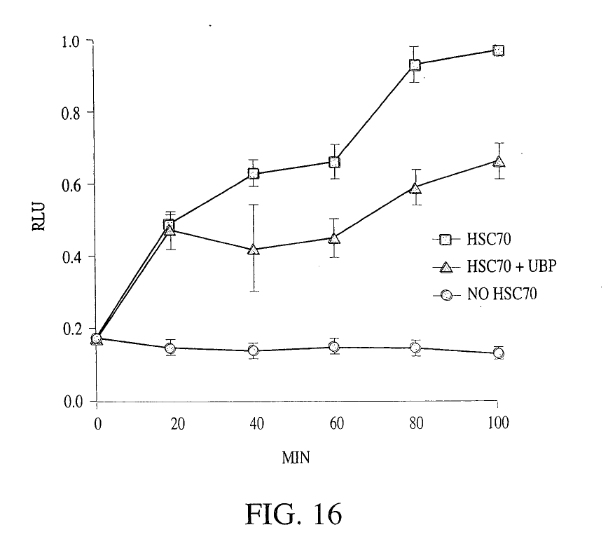

In addition, in Example 3 the present disclosure illustrates that a cellular protein (UBP) known to bind to the viral particle release regulating HIV-1 protein Vpu and inhibit virus replication, can also bind to cellular proteins, namely hsp70, hsc70, and hsp90. The data establish that UBP negatively regulates HSP function by decreasing HSP ATPase activity and substrate folding activity (FIGS. 15 and 16). In addition, the invention discloses that the tetratricopeptide repeats in the N-terminal half of UBP are necessary and sufficient for UBP-hsp70 interaction. Thus, the invention illustrates that UBP can play a role as a co-chaperone. The present further discloses that the viral protein Vpu functions at a later step than does UBP. In addition, the data establish that HIV Gag protein forms a co-complex with UBP and hsp70. Therefore, the disclosure presented herein establishes important roles for hsp40, hsp70, and hsp90 in virus replication and further establishes that methods described herein to inhibit these or other HSPs or their regulation are effective in inhibiting virus replication. The invention also establishes that the HIV-1 Gag protein plays a role in HSP activation and regulation or is modified by the action of HSPs. Moreover, the data establish that cellular-cellular protein interactions as well as viral-cellular protein interactions regulate virus replication. Thus, the invention provides methods of inhibiting virus replication by inhibiting cellular HSP pathways, functions, or interactions.

Without wishing to be bound by theory, the following are evident: numerous viruses, including bacteriophage, induce a heat shock response, so this is a widely used strategy for virus replication. Because the HSPs are involved in a potent immune response, there is enormous evolutionary “pressure” for viruses to move away from containing genes that induce expression of HSPs. The fact that they have not, and the fact that this HSP induction is shared by viruses across species from bacteria, to chickens, to humans, suggests that viruses may be reliant on cellular HSPs for critical points in their replication.

The present invention also relates to methods of identifying compounds which inhibit heat shock dependent virus replication. The invention encompasses the idea that modulating the cellular HSP response will be useful in inhibiting virus replication, and that a whole new class of antiviral compounds can be developed as a result of this discovery, e.g., those which block virus replication by inhibiting HSPs or HSP pathways. Not wishing to be bound by theory, it can be theorized that when an antiviral compound targets an essential cellular function, it is less likely that drug resistant virus strains will evolve and thus evade drug therapy.

The present invention includes novel mechanisms for the development of new antiviral compounds which target cellular functions essential for virus replication. The invention also includes novel antiviral compounds and methods of their use. These antiviral compounds are designed primarily to disrupt cellular protein interactions, in particular those interactions in the heat shock protein pathway. However, as is described in greater detail below, the invention also encompasses methods of inhibiting heat shock protein function and pathways as well as the identification and use of compounds which inhibit heat shock protein pathways.

Methods of Inhibiting Heat Shock Protein Dependent Virus Replication

The disclosure provides herein methods for inhibiting heat shock protein dependent virus replication specifically provided are examples using adenovirus, hantavirus, and HIV-1. Each aspect of the disclosure should be construed to apply to other viruses as well.

Inhibiting Heat Shock Protein Interactions with Proteins or Peptides

It has been discovered in the present invention that hsp40 is required for replication by the adenovirus tested herein and therefore a likely target for antiviral compounds is hsp40. Hsp40 is a known cofactor of hsp70; while manipulation of hsp40 alone may not have major detrimental effects on the cell, modulation of this protein affects virus replication. Viruses known to stimulate HSPs or to interact with HSPs during replication include adenovirus, herpes simplex virus, measles virus, Newcastle's disease virus, papilloma virus, respiratory syncitial virus (RSV), simian virus 40 (SV40), and cytomegalovirus. Thus, blocking or manipulating hsp40 to prevent proper interaction of hsp40 with hsp70, or blocking or manipulating other HSPs, is expected to inhibit the replication of a wide variety of viruses. Given these examples, the fact that HSPs can be induced in response to stress, and the fact that virus infection is inherently stressful to the cell, it is to be expected that many other viruses will also induce and require an HSP response.

By “heat shock protein interaction” is meant a functional relationship of one HSP with another HSP, or of an HSP with a component of its heat shock response pathway, and includes, but is not limited to, binding of one type of HSP to another type of HSP, such as, but not limited to, binding of hsp40 with hsp70. The interaction results in a modification of an activity or the gain or loss of activity. For example, it is known that hsp40 is a cofactor of hsp70 and that the hsp40 J domain appears to be required for the protein-protein interaction. The resulting interaction enhances hsp70 function. The invention should be construed to include other proteins, such as UBP (described below in Example 3), and other HSPs. While the present disclosure primarily focuses on hsp40 and hsp70, the disruption of the interaction of these two proteins should be construed as merely one example of the power of the present invention and the invention should therefore not be construed to be limited solely to the examples provided herein.

Accordingly, the invention includes methods that interfere with the interaction of hsp40 with hsp70, including, but not limited to, using an hsp40 J domain as an inhibitor of interaction of hsp40 with hsp70. Methods of using an hsp40 J domain as an inhibitor of the interaction of hsp40 with hsp70 include, but are not limited to, the following methods. Any transfection or infection method known to those skilled in the art can be used to introduce an expression vector comprising an hsp40 sequence. Appropriate expression vectors include a recombinant virus such as CELO or adenovirus type 5, vaccinia virus, retrovirus, semliki forest virus, baculovirus, and Epstein-Barr virus, or any other suitable recombinant virus system or expression vector system.

A preferred embodiment of the invention is the use of an hsp40 J domain as an inhibitor of hsp40-hsp70 interaction. The invention includes the human hsp40 J domain peptide from about amino acid 1 to amino acid 70 (SEQ ID NO:1). The amino acid sequence corresponding to the hsp40 J domain is about:

(SEQ ID NO:1)

MGKDYYQTLGLARGASDEEIKRAYRRQALRYHPDKNKEPGAEEKFKEIAE
AYDVLSDPRKREIFDRYGEE.

The accession number for the human hsp40 protein is SWISS-PROT accession number P25685 (SEQ ID NO:2) and for the gene it is GenBank accession number D85429 (SEQ ID NO:3).

The methods of the invention should not be construed to mean that an hsp40 J domain peptide is the only peptide or protein capable of inhibiting HSP function. This invention should be construed to include the use of other proteins or peptides that inhibit the interaction of hsp40 with hsp70 or the interaction of other HSPs with each other, when the interaction is required for virus replication. Based upon the discoveries described herein, overexpression of other wild-type or mutant proteins in the HSP pathway is expected to interfere with HSP functions, including mutations or deletions in hsp40 or hsp70 such as a deletion of the C-terminal acidic motif. In addition, overexpression of an HSP or another component of an HSP pathway may also lead to inhibition of HSP functions, perhaps by titrating essential, limiting components of the pathway. The invention should therefore also be construed to include methods of inhibiting other heat shock proteins, such as hsp27, hsc70, and hsp90α and their interactions with each other, or with other cellular proteins.

In another aspect, the invention includes a peptide, derivative, or fragment thereof, having at least about 30% homology with the hsp40 J domain amino acid sequence of SEQ ID NO:1. Preferably the peptide is about 35% homologous, more preferably the peptide is about 40% homologous, more preferably the peptide is about 45% homologous, more preferably the peptide is about 50% homologous, more preferably the peptide is about 60% homologous, more preferably the peptide is about 70% homologous, more preferably the peptide is about 90% homologous, more preferably the peptide is about 95% homologous, and even more preferably about 99% homologous with the hsp40 J domain amino acid sequence of SEQ ID NO:1.

One skilled in the art would appreciate, based upon the disclosure provided herein, that modified gene sequences, i.e. genes having sequences that differ from the gene sequences encoding the naturally-occurring protein, are also encompassed by the invention, so long as the modified gene still encodes a protein that functions to inhibit heat shock protein dependent virus replication in any direct or indirect manner. These modified gene sequences include modifications caused by point mutations, modifications due to the degeneracy of the genetic code or naturally occurring allelic variants, and further modifications that have been introduced by genetic engineering, i.e., by the hand of man.

Techniques for introducing changes in nucleotide sequences that are designed to alter the functional properties of the encoded proteins or polypeptides are well known in the art. Such modifications include the deletion, insertion, or substitution of bases, and thus, changes in the amino acid sequence. Changes may be made to increase the activity of a protein, to increase its biological stability or half-life, to change its glycosylation pattern, and the like. All such modifications to the nucleotide sequences encoding such proteins are encompassed by this invention.

The skilled artisan would appreciate, based on the disclosure herein, that a non-heat shock protein or co-chaperone protein which interacts with an HSP can be used to inhibit an HSP function or HSP-HSP interaction that is required for virus replication. Overexpression of such a non-heat shock protein or co-chaperone protein is expected to interfere with HSP function as described about. The skilled artisan would also appreciate that HSP modification of viral proteins is expected to be important and that interfering with that modification would also be expected to affect virus replication. Furthermore, the skilled artisan would appreciate that fragments or portions of inhibitory proteins or peptides which interact with an HSP can be utilized to inhibit HSP function or interaction. In addition, the skilled artisan would appreciate that mimetics of inhibitors can be created using techniques known in the art. These mimetics can be created to have similar structural and binding properties to inhibitors discovered to have the ability to inhibit heat shock dependent virus replication.

The invention discloses methods for inhibiting heat shock protein dependent HIV-1 replication by inhibiting HSP interactions with other HSPs or non-HSP proteins and peptides. The general methods of the invention useful for inhibiting heat shock protein interactions are outlined for adenovirus in detail above. The general method applies to hantavirus and HIV-1 as well.

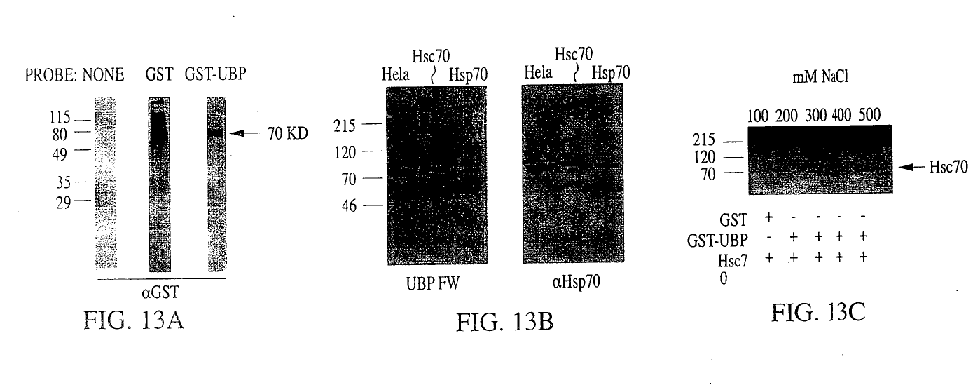

In one aspect of the invention, the cellular viral particle u binding protein UBP can be used to inhibit hsp70 activity. In another aspect of the invention UBP can be used to inhibit other HSP functions. By way of example, FIGS. 13-21 illustrate that UBP, which is known to inhibit HIV-1 viral particle release, binds to hsp70 and reduces its activity. Furthermore, UBP binds to other HSPs, including hsp90 and hsc70, as well as to the viral protein Gag.

Inhibiting Heat Shock Protein Function Using Recombinant Cell or Transgenic Techniques

In another embodiment, transgenic techniques can be used to derive a transgenic animal, the gen cells of which comprise an hsp40 J domain transgene or transgenes for other proteins or peptides discovered to have heat shock protein inhibitory activity. This technique can be used to make, for example, a virus-resistant livestock strain.

The invention includes a recombinant cell comprising, inter alia, an isolated nucleic acid encoding hsp40 J domain, an antisense nucleic acid complementary thereto, a nucleic acid encoding an antibody that specifically binds hsp40 J domain, and the like. In one aspect, the recombinant cell can be transiently transfected with a plasmid encoding a portion of the nucleic acid encoding hsp40 J domain. The nucleic acid need not be integrated into the cell genome nor does it need to be expressed in the cell. Moreover, the cell may be a prokaryotic or a eukaryotic cell and the invention should not be construed to be limited to any particular cell line or cell type. Such cells include, but are not limited to, fibroblasts, hepatocytes, skeletal muscle cells, and adipocytes.

In one aspect, the recombinant cell comprising an isolated nucleic acid encoding mammalian hsp40 J domain is used to produce a transgenic non-human mammal. That is, the exogenous nucleic acid, or transgene as it is also referred to herein, is introduced into a cell, and the cell is then used to generate the non-human transgenic mammal. The cell into which the transgene is introduced is preferably an embryonic stem (ES) cell. However, the invention should not be construed to be limited solely to ES cells comprising the transgene of the invention nor to cells used to produce transgenic animals. Rather, a transgenic cell of the invention includes, but is not limited to, any cell derived from a transgenic animal comprising a transgene, a cell comprising the transgene derived from a chimeric animal derived from the transgenic ES cell, and any other cell or ES cell comprising the transgene which may or may not be used to generate a non-human transgenic mammal.

Further, it is important to note that the purpose of transgene-comprising, i.e., recombinant, cells should not be construed to be limited to the generation of transgenic mammals. Rather, the invention should be construed to include any cell type into which a nucleic acid encoding a mammalian HSP or regulator of an HSP is introduced, including, without limitation, a prokaryotic cell and a eukaryotic cell comprising an isolated nucleic acid encoding mammalian hsp40 J domain. The invention should not be construed to be limited solely to hsp40, but should be construed to include other HSPs as well as other proteins, such as UBP (see HIV-1 in Example 3, below). It will be appreciated by those of skill in the art that benefit can be obtained from a sense or antisense configuration, depending on the particular function that is being targeted.

Such a cell expressing an isolated nucleic acid encoding an hsp40 J domain can be used to provide hsp40 J domain peptide to a cell, tissue, or whole animal where a higher level of hsp40 J domain competes with endogenous hsp40 and can be useful to treat or alleviate a disease, disorder or condition associated with expression and/or activity of endogenous activity. Such diseases, disorders or conditions can include, but are not limited to the viral associated diseases described herein. Therefore, the invention includes a cell expressing hsp40 J domain to decrease hsp40 and hsp70 interaction and/or activity, where increasing hsp40 J domain peptide expression, protein level, and/or activity can be useful to treat or alleviate a disease, disorder or condition.

As noted herein, the invention includes a non-human transgenic mammal comprising an exogenous nucleic acid inserted into a desired site in the genome thereof thereby deleting the coding region of a desired endogenous target gene, i.e., a knock-out transgenic mammal. Further, the invention includes a transgenic non-human mammal wherein an exogenous nucleic acid encoding hsp40 J domain is inserted into a site the genome, i.e., a “knock-in” transgenic mammal. The knock-in transgene inserted may comprise various nucleic acids encoding, for example, a tag polypeptide, a promoter/regulatory region operably linked to the nucleic acid encoding hsp40 J domain not normally present in the cell or not typically operably linked to hsp40 J domain. The invention should not be construed to include solely a recombinant cell or transgenic animal comprising an hsp40 J domain, but should be construed to include other HSPs as well as other non-HSP peptides which regulate HSPs, including UBP.

The generation of the non-human transgenic mammal of the invention is preferably accomplished using the method which is now described. However, the invention should in no way be construed as being limited solely to the use of this method, in that, other methods can be used to generate the desired knock-out or knock-in mammal.

In the preferred method of generating a non-human transgenic mammal, ES cells are generated comprising the transgene of the invention and the cells are then used to generate the knock-out animal essentially as described in Nagy and Rossant (1993, In: Gene Targeting, A Practical Approach, pp.146-179, Joyner ed., IRL Press). ES cells behave as normal embryonic cells if they are returned to the embryonic environment by injection into a host blastocyst or aggregate with blastomere stage embryos. When so returned, the cells have the full potential to develop along all lineages of the embryo. Thus, it is possible, to obtain ES cells, introduce a desired DNA therein, and then return the cell to the embryonic environment for development into mature mammalian cells, wherein the desired DNA may be expressed.

Precise protocols for the generation of transgenic mice are disclosed in Nagy and Rossant (1993, In: Gene Targeting, A Practical Approach, Joyner ed. IRL Press, pp. 146-179) and are therefore not repeated herein. Transfection or transduction of ES cells in order to introduce the desired DNA therein is accomplished using standard protocols, such as those described, for example, in Sambrook et al. (1989, Molecular Cloning: A Laboratory Manual, Cold Spring Harbor Laboratory, New York), and in Ausubel et al. (1997, Current Protocols in Molecular Biology, John Wiley & Sons, New York). Preferably, the desired DNA contained within the transgene of the invention is electroporated into ES cells, and the cells are propagated as described in Soriano et al. (1991, Cell 64:693-702).

The transgenic mammal of the invention can be any species of non-human mammal. Thus, the invention should be construed to include generation of transgenic mammals encoding the chimeric nucleic acid, which mammals include mice, hamsters, rats, rabbits, pigs, sheep and cattle. The methods described herein for generation of transgenic mice can be analogously applied using any mammalian species. Preferably, the transgenic mammal of the invention is a rodent and even more preferably, the transgenic mammal of the invention is a mouse.

To identify the transgenic mammals of the invention, pups are examined for the presence of the isolated nucleic acid using standard technology such as Southern blot hybridization, PCR, and/or RT-PCR, or the presence of the protein or peptide can be detected by techniques known to those of skill in the art.

Alternatively, recombinant cells expressing an hsp40 J domain peptide can be administered in ex vivo and in vivo therapies where administering the recombinant cells thereby administers the protein to a cell, a tissue, and/or an animal. Additionally, the recombinant cells are useful for the discovery of hsp40 J domain signaling pathways. This technique is also useful for determining other HSP signaling pathways.

Those of skill in the art will appreciate that other uses exist for recombinant cells and transgenic animals.

To prevent toxicity from expression of the inhibitor outside of the context of virus infection, the expression of the inhibitor can be placed under the control of transcriptional elements known to be specifically up-regulated during infection by the pathogen. For example, expression of the inhibitor could be controlled by the HIV LTRJTAR which is strongly upregulated by TAT during HIV infection.

Expression of a multimer of a protein or a peptide with hsp40 J domain properties or with the properties of other heat shock proteins, derivative, or fragments could alter the interactions between hsp40 and hsp70. Furthermore, synthetic peptides encompassing the J domain, or any other heat shock protein interaction-inhibiting peptides, and, optionally including peptide sequences that facilitate intracellular uptake of such peptides e.g., proteins or peptides derived from HSV VP22, HIV TAT or other peptides demonstrated to promote intracellular delivery could be used to inhibit hsp40 interaction with hsp70.

Using Antibodies to Inhibit Heat Shock Protein Dependent Virus Replication

The invention also includes a method by which antibodies can be generated and used as inhibitors of heat shock protein interactions and function wherein virus replication is heat shock protein-dependent. The preparation and use of antibodies to inhibit protein function is a technique known by those skilled in the art. The generation of polyclonal antibodies is accomplished by inoculating the desired animal with the antigen and isolating antibodies which specifically bind the antigen therefrom.

Monoclonal antibodies can be used effectively intracellularly to avoid uptake problems by cloning the gene and then transfecting the gene encoding the antibody. Such a nucleic acid encoding the monoclonal antibody gene obtained using the procedures described herein may be cloned and sequenced using technology which is available in the art.

Monoclonal antibodies directed against full length or peptide fragments of a protein or peptide may be prepared using any well known monoclonal antibody preparation procedure. Quantities of the desired peptide may also be synthesized using chemical synthesis technology. Alternatively, DNA encoding the desired peptide may be cloned and expressed from an appropriate promoter sequence in cells suitable for the generation of large quantities of peptide. Monoclonal antibodies directed against the peptide are generated from mice immunized with the peptide using standard procedures as referenced herein. A nucleic acid encoding the monoclonal antibody obtained using the procedures described herein may be cloned and sequenced using technology which is available in the art. Further, the antibody of the invention may be “humanized” using the existing technology known in the art. In another aspect, antisense nucleic acids complementary to HSP mRNAs can be used to block HSP function by inhibiting translation of an HSP and this can be done by transfecting a gene with the appropriate sequence linked to a promoter to control its expression. HSP genes have been sequenced and based on this data antisense nucleic acids can be readily prepared using techniques known to those skilled in the art.

To generate a phage antibody library, a cDNA library is first obtained from mRNA which is isolated from cells, e.g., the hybridoma, which express the desired protein to be expressed on the phage surface, e.g., the desired antibody. cDNA copies of the mRNA are produced using reverse transcriptase. cDNA which specifies immunoglobulin fragments are obtained by PCR and the resulting DNA is cloned into a suitable bacteriophage vector to generate a bacteriophage DNA library comprising DNA specifying immunoglobulin genes. The procedures for making a bacteriophage library comprising heterologous DNA are well known in the art and are described, for example, in Sambrook et al. (1989, Molecular Cloning: A Laboratory Manual, Cold Spring Harbor, N.Y.).

Bacteriophage which encode the desired antibody, may be engineered such that the protein is displayed on the surface thereof in such a manner that it is available for binding to its corresponding binding protein, e.g., the antigen against which the antibody is directed. Thus, when bacteriophage which express a specific antibody are incubated in the presence of a cell which expresses the corresponding antigen, the bacteriophage will bind to the cell. Bacteriophage which do not express the antibody will not bind to the cell. Such panning techniques are well known in the art and are described for example, in Wright et al., (supra).

Processes such as those described above, have been developed for the production of human antibodies using M13 bacteriophage display (Burton et al., 1994, Adv. Immunol. 57:191-280). Essentially, a cDNA library is generated from mRNA obtained from a population of antibody-producing cells. The mRNA encodes rearranged immunoglobulin genes and thus, the cDNA encodes the same. Amplified cDNA is cloned into M13 expression vectors creating a library of phage which express human Fab fragments on their surface. Phage which display the antibody of interest are selected by antigen binding and are propagated in bacteria to produce soluble human Fab immunoglobulin. Thus, in contrast to conventional monoclonal antibody synthesis, this procedure immortalizes DNA encoding human immunoglobulin rather than cells which express human immunoglobulin.

The procedures just presented describe the generation of phage which encode the Fab portion of an antibody molecule. However, the invention should not be construed to be limited solely to the generation of phage encoding Fab antibodies. Rather, phage which encode single chain antibodies (scFv/phage antibody libraries) are also included in the invention. Fab molecules comprise the entire Ig light chain, that is, they comprise both the variable and constant region of the light chain, but include only the variable region and first constant region domain (CH1) of the heavy chain. Single chain antibody molecules comprise a single chain of protein comprising the Ig Fv fragment. An Ig Fv fragment includes only the variable regions of the heavy and light chains of the antibody, having no constant region contained therein. Phage libraries comprising scFv DNA may be generated following the procedures described in Marks et al., 1991, J. Mol. Biol. 222:581-597. Panning of phage so generated for the isolation of a desired antibody is conducted in a manner similar to that described for phage libraries comprising Fab DNA.

The invention should also be construed to include synthetic phage display libraries in which the heavy and light chain variable regions may be synthesized such that they include nearly all possible specificities (Barbas, 1995, Nature Medicine 1:837-839; de Kruif et al. 1995, J. Mol. Biol. 248:97-105).

By the term “synthetic antibody” as used herein, is meant an antibody which is generated using recombinant DNA technology, such as, for example, an antibody expressed by a bacteriophage as described herein. The term should also be construed to mean an antibody which has been generated by the synthesis of a DNA molecule encoding the antibody and which DNA molecule expresses an antibody protein, or an amino acid sequence specifying the antibody, wherein the DNA or amino acid sequence has been obtained using synthetic DNA or amino acid sequence technology which is available and well known in the art.

Inhibiting Heat Shock Protein Function Using Antisense Techniques

In one embodiment, antisense nucleic acids complementary to hsp40 mRNA or hsp70 mRNA can be used to block the expression or translation of the corresponding mRNAs. Antisense oligonucleotides as well as expression vectors comprising antisense nucleic acids complementary to nucleic acids encoding HSPs can be prepared and used based on techniques routinely performed by those of skill in the art. The antisense oligonucleotides of the invention include, but are not limited to, phosphorothioate oligonucleotides and other modifications of oligonucleotides. Methods for synthesizing oligonucleotides, phosphorothioate oligonucleotides, and otherwise modified oligonucleotides are well known in the art (U.S. Pat. No. 5,034,506; Nielsen et al., 1991, Science 254: 1497) This invention should not be construed to include only hsp40 or hsp70 and should not be construed to include only these particular antisense methods described.

Oligonucleotides which contain at least one phosphorothioate modification are known to confer upon the oligonucleotide enhanced resistance to nucleases. Specific examples of modified oligonucleotides include those which contain phosphorothioate, phosphotrioester, methyl phosphonate, short chain alkyl or cycloalkyl intersugar linkages, or short chain heteroatomic or heterocyclic intersugar (“backbone”) linkages. In addition, oligonucleotides having morpholino backbone structures (U.S. Pat. No. 5,034,506) or polyamide backbone structures (Nielsen et al., 1991, Science 254: 1497) may also be used.

The examples of oligonucleotide modifications described herein are not exhaustive and it is understood that the invention includes additional modifications of the antisense oligonucleotides of the invention which modifications serve to enhance the therapeutic properties of the antisense oligonucleotide without appreciable alteration of the basic sequence of the antisense oligonucleotide.

Alignment of the inducible hsp70 gene family member hsp72 in disparate mammal species (African green monkey, rat, man, and cow) shows that there is a 26-bp consensus sequence that begins 21 nt 3′ of the transcription start site. The position and length of this sequence make it an ideal target for translational inhibition by an oligonucleotide. By comparison, the sequence of the African green monkey hsc70 (constitutively expressed) gene in this region differs at 4 positions, and should not be subject to inhibition by the presence of the hsp72-specific oligonucleotides, since even 1 nucleotide difference is enough to permit sequence specificity (Chang et al., 1991, Biochemistry 30:8283-8286; Stein and Chang, 1993, Science 261:1004-1112). In fact, the comparable region of hsc70 is also closely conserved and oligonucleotides inhibitors for hsc70 can be used as well so that the effects of hsp72 and hsc70 (hsp73) can be compared. Phosphorothioate oligonucleotides, which have very low sensitivity to nuclease degradation, will be used. The oligonucleotides in question also lack CG motifs, which should help reduce toxicity for in vivo use.

The oligonucleotide inhibitors of hsp70 and hsc70 can be used independently in the cell culture system essentially as described herein for quercetin and hantavirus (Example 2). The phosphorothioate oligonucleotides enter cells readily without the need for transfection or electroporation, which avoids subjecting the cells to nonspecific inducers of a stress response that might confound the experiment. Effective inhibitory concentrations for phosphorothioates range between 1 and 50 μM, so a titration curve for diminution of HSP signal in western blots will be done. Once inside the cells, the PT-oligonucleotides hybridize with the nascent mRNA very close to the transcriptional start site, which is usually a good site for maximum effect of antisense oligonucleotide inhibition.

Pre-existing hsp72 and hsc70 may be sufficient for Sin Nombre (SN) virus (hantavirus) infection to proceed at normal kinetics, but the effectiveness of quercetin and specific antisense inhibitors of both molecules is expected to be helpful in dissecting the role of those proteins. The same may be true for other viruses as well. The ability to selectively inhibit transcription of hsc70, hsp70, or both genes with antisense molecules, is expected to also inhibit the induction of increased SN virus replication in persistently infected cells. Thus, the invention provides methods for the use of antisense oligonucleotides that will be effective at diminishing steady-state levels of the protein of interest, and that inhibition of hsp70 or other important HSPs will reduce steady-state synthesis of virions as assessed by expression of N antigen, infectious virus, and RNA in the supernatant.

Suppression of intracellular hsp72 expression is compatible with cell viability, although it may reduce viability in the face of a heat stress. One of skill in the art will know that it may be necessary to titrate the heat stress to avoid excessive cytotoxicity before testing the depression of SN virus, or other viruses, in persistently infected cells, or the effects of the inhibition on the permissiveness of Vero E6 cells to SN virus infection. The invention should not be construed to be limited solely to hantavirus infection and should be construed to include all other viruses which are dependent upon heat shock proteins for replication and the cells which they infect.

Inhibiting Heat Shock Proteins or Heat Shock Protein Pathways

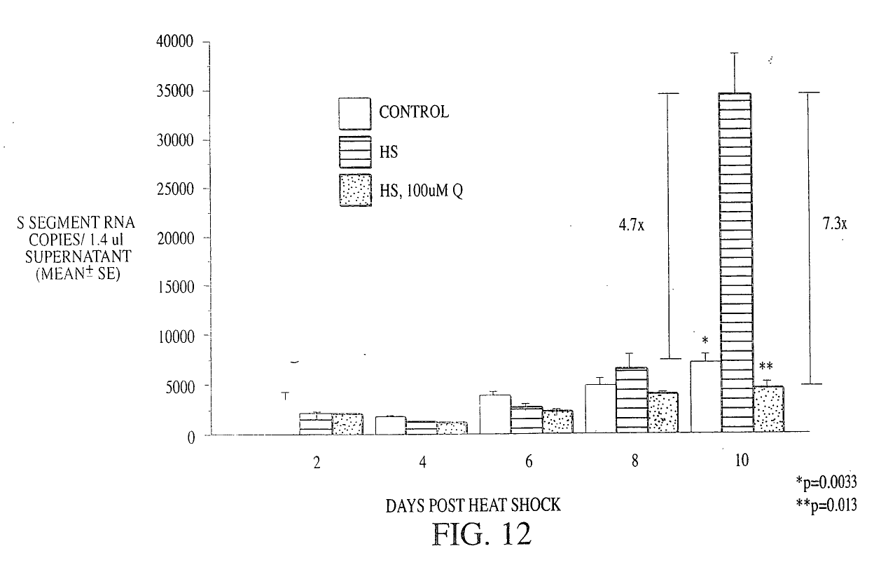

The invention described herein also suggests that hsp70 is required for hantavirus reactivation. The invention also relates to the inhibition of HSPs or HSP pathways by flavonoids. Flavonoids are any of a large group of aromatic oxygen heterocyclic compounds that are widely distributed in higher plants. The flavonoid quercetin, a known inhibitor of hsp70 induction, is shown herein to inhibit heat shock induced reactivation of hantavirus. Based on the adenovirus data described above and because hsp40 interaction with hsp70 enhances the efficiency of the complex, inhibiting hsp40 should be effective in inhibiting hantavirus replication. Methods of inhibiting hsp70 should inhibit hantavirus, as shown by the quercetin data of Example 2 and FIG. 12. The method is not limited to the viruses described herein (adenoviruses, hantavinises, and HIV), and should be construed to include other viruses as well.

In one embodiment, the compounds used for their ability to inhibit heat shock protein dependent virus replication include flavonoids such as quercetin. The invention includes assays for their use, as generally described in Example 2 and FIG. 12.

In one aspect of the invention, quercetin can be administered at concentrations of 1 nM to 1 M to virus-infected cells to inhibit heat shock protein dependent virus replication. In another aspect quercetin can be added at concentrations of 100 nM to 500 μM. In yet another aspect quercetin can be administered at concentrations of 1 μM to 250 μM. One of skill in the art call easily titrate the amount of quercetin to be used.

In one embodiment, after quercetin has been administered to virus-infected cells, heat shock protein function and virus replication can be determined. In one aspect, the amount of hsp70 mRNA present following quercetin treatment of virus infected cells can be determined. In another aspect, the heat shock protein function to be measured is the amount of hsp70 protein that is present, which is all indirect measurement of hsp70 function. In yet another aspect, hsp70 function can be measured more directly by measuring its ATPase activity, transport activity, or folding activity. In one aspect, virus replication can be measured by release of virus antigen, such as N antigen. By way of example viral RNA titers can be measured to determine the effect of quercetin.

In one embodiment quercetin is added to virus-infected Vero E6 cells. It should not be construed that Vero E6 cells are the only cells to which quercetin can be administered to inhibit virus replication. It should be construed that quercetin can be added to other cell types and species of cells as well. It should also be construed that quercetin can be administered in vivo. Quercetin can be administered to birds as wells as to mammals. Preferably the mammal is a human.

The invention should not be construed to be limited solely to the flavonoids described herein, but should be construed to include all other flavonoids and structurally related compounds as well, including naringenin, naringin, morin, catechin, kaempferol, myricetin, phloretin, phlorizdin, rutin, and 3-methylquercetin. The invention should also not be construed to be limited solely to the assays described herein, but should be construed to include all other virus replication and HSP function assays as well. Moreover, the invention should not be construed to solely encompass quercetin, but instead should include other small molecules identified using the assays described herein.

The method of the invention is useful for inhibiting virus replication in cells or animals infected with a virus that is dependent upon heat shock proteins or heat shock responses for its replication. The method is not limited to the cells described herein, and should be construed to include avian as well as mammalian cells. The method should also be construed to include livestock, pets and humans.

In one embodiment, the viruses being inhibited by inhibitors of heat shock protein dependent virus replication include adenoviruses, hantaviruses, parvoviruses, and HIV-1. The invention should not be construed to be limited solely to the inhibition of these viruses, but should be construed to include all viruses whose replication is heat shock protein dependent.

The method of the invention is useful for inhibiting virus replication by inhibiting cellular HSPs, including hsp27, hsp40, hsp70 and hsp90α. The heat shock proteins listed herein should be construed to include all the members of the families of each, including constitutive and inducible forms. For example, the need for molecular chaperone function in unstressed cells is met by constitutive forms of HSPs such as hsc70, a constitutively expressed form of hsp70.

“Heat shock protein inhibitor,” as used herein, refers to any agent, the application of which results in the inhibition of a heat shock protein function or heat shock pathway function. “HSP function” as used herein should be construed to comprise the interaction of one HSP with another HSP, the interaction of an HSP with a non-HSP, or any function of an HSP that is required for or enhances virus replication. Inhibition of function can be direct, such as in the case of an inhibitor that directly inhibits a required interaction of two heat shock proteins or that directly inhibits the action or function of a single heat shock protein.

Inhibition of HSP function can also be indirect, such as inhibiting the synthesis or secondary modifications of a heat shock protein or its mRNA, or inhibiting the pathway by which a heat shock protein elicits its effect. In mammalian cells, HSPs can be regulated at the level of transcription by the heat shock factor HSF1 (Lis and Wu, 1993, Cell 74:1-4). By way of example, a heat shock protein interaction inhibitor can be an isolated nucleic acid, an antisense nucleic acid, an antiviral agent, an antibody, a protein, a peptide, a synthetic peptide, a cytokine, or other compounds or agents such as small molecules. An inhibitor should not be construed to be limited to being derived only from the aforementioned classes of molecules. Methods for using or developing an inhibitor are described herein or are known to those skilled in the art.

It will be recognized by one of skill in the art that the various embodiments of the invention as described above relating to adenovirus, also encompass other viruses, including hantavirus and HIV-1. Furthermore, the embodiments of the invention described herein for hantavirus apply to adenovirus as disclosed above and in Example 1, as well as to HIV-1 as disclosed below in Example 3. Thus, it should not be construed that the embodiments described herein for adenovirus or HIV-1 or hantavirus do not apply to each of the other viruses disclosed herein.

Methods of Identifying Compounds which Inhibit HSP Dependent Virus Replication

The invention includes a method of identifying compounds that can be used as antiviral agents. This includes, but is not limited to, a method of identifying compounds which inhibit heat shock protein dependent virus replication in cells infected with virus. Another aspect of the invention includes more specifically, a method for identifying compounds which inhibit a heat shock protein interaction which is required for virus replication. The method includes techniques for screening effects of compounds on heat shock protein interaction and virus replication and for identifying compounds which produce these effects. Virus replication can be measured using various assays known to those skilled in the art. The invention also includes a method of identifying compounds which inhibit heat shock protein dependent virus replication in animals. Preferably the animal is a human.

The invention discloses herein methods for measuring heat shock protein interactions and heat shock protein function, as well as various methods for measuring virus replication. In addition, methods for analyzing the results of the various types of assays in conjunction with one another are included to demonstrate the effect of an inhibitor of heat shock protein dependent virus replication.

In one aspect the method used for screening inhibitors of heat shock protein dependent virus replication includes assays to measure protein folding activity or ATPase activity of an HSP, coupled with an assay to measure virus replication, which can include quantitative focus assays, TaqMan RT-PCR assays, or virion/antigen release assays, as detailed in Examples 1-3.

In one embodiment the method used for identifying inhibitors of heat shock dependent virus replication includes selecting proteins which stably bind to hsp40 or hsp70 in a far-western analysis. By way of example, the cellular protein UBP, which inhibits HIV-1 viral particle release, was shown in Example 3 by far-western analysis to bind to hsp70. The method should be construed to include identifying proteins which stably bind to other HSPs as well.

In another embodiment, the method used for identifying proteins that interact with or inhibit an HSP includes using a yeast two hybrid screen for proteins that interact with the HSP. This technique cain be applied by those well skilled in the art as outlined in Example 3 for HIV-1 regulation.

In yet another embodiment, the method used for identifying proteins that interact with hsp40 or hsp70 includes using co-immunoprecipitation techniques. For example, it would be known to one of skill in the art that using an antibody against an HSP, the HSP can be precipitated and one of skill in the art can establish conditions by which another protein which interacts with the HSP is precipitated as well.

In one aspect the identified compounds include proteins and peptides and mutants, derivatives and fragments, thereof.

In yet another aspect, the invention includes the identification of compounds, including, but not limited to, small molecules, drugs or other agents, for their ability to disrupt HSP functions or the interaction of one HSP with another HSP. For example, high throughput screens can be established to identify small molecules that inhibit hsp40/hsp70 binding. This assay can be based on the refolding of luciferase which is known to be influenced by the interaction of hsp40 with hsp70. The invention should not be construed to include the use of assays to identify only inhibitors of hsp40/hsp70 interactions, but should be construed to include assays to identify inhibitors of other heat shock protein interactions as well.

In one embodiment, the compounds screened for their ability to inhibit heat shock protein dependent virus replication include flavonoids such as quercetin. The invention includes assays, as generally described in Example 2 and FIG. 12. The invention should not be construed to be limited solely to the flavonoids described herein, but should be construed to include all other flavonoids and structurally related compounds as well, including naringenin, naringin, morin, catechin, kaempferol, myricetin, phloretin, phlorizdin, rutin, and 3-methylquercetin. The invention should also not be construed to be limited solely to the assays described herein, but should be construed to include all other virus replication and HSP function assays as well.

In one aspect of the invention, an assay can be performed in which the non-heat shock protein UBP, or mutants, fragments or derivatives thereof, can be used to interact with an HSP and its ability to inhibit HSP dependent virus replication would be correlated with its ability to reduce HSP ATPase activity and/or HSP refolding activity. This assay can be used to test modification of UBP or it can be used to measure the effects of candidate inhibitors of heat shock protein dependent virus replication as described above, and in further detail below.

UBP is a member of the tetratricopeptide (TPR) protein family. In another aspect of the invention, a different member of the TPR family other than UBP can be used. The invention should be construed to include other members of the TPR family as well as other assays to measure HSP activity and function. By way of example, HIV-1 methods of the invention are disclosed in Example 3 and FIGS. 13-21.

A compound identified as an inhibitor of heat shock protein-dependent virus replication by the present invention can be administered to any animal, including a human. The compound or known inhibitor may be administered via any suitable mode of administration, such as intramuscular, oral, subcutaneous, intradermal, intravaginal, rectal, or intranasal administration. The preferred modes of administration are oral, intravenous, subcutaneous, intramuscular or intradermal administration. The most preferred mode is subcutaneous administration. The invention contemplates the use of an inhibitor of heat shock protein-dependent virus replication to inhibit virus replication in animals. Preferably the animal is a human.

Assays for Testing Inhibitors of Heat Shock Protein Function and Interaction

The present disclosure establishes a series of assays for identifying inhibitors of on heat shock protein function and heat shock protein interactions and for inhibitors of virus replication. These assays can be then be used in conjunction with one another to identify and assay for the inhibitors which inhibit heat shock protein dependent virus replication. All of the cellular, biochemical and molecular assays described herein should be construed to be useful for the invention.

In one aspect, the invention discloses assays for measuring the effects of inhibitors on levels of HSPs both in vivo and in vitro. These assays include sampling cells, conditioned media, tissues, and blood.

In one embodiment HSPs are measured by western blot analyses. Included with these analyses are various techniques described herein such as immunoprecipitation and co-immunoprecipitation. In one aspect the invention includes far-western analyses, as described in Example 3. In yet another aspect of the invention ELISA assays can be used to measure protein levels in the presence or absence of a candidate inhibitor of heat shock protein dependent virus replication. The invention also includes immunohistochemical and immunofluorescence assays to compare HSP levels in the presence or absence of a candidate inhibitor.

In another embodiment the function or activity of an HSP can be measured to identify the effects of candidate inhibitors of heat shock protein dependent virus replication. The present disclosure provides for assays to measure function which include binding ability, ATPase activity, ability to fold other proteins, and the ability to support virus replication. The invention should not be construed to be limited to measuring the function or activity of only one HSP, but should be construed to include assays to measure functions or activities of other HSPs as well.

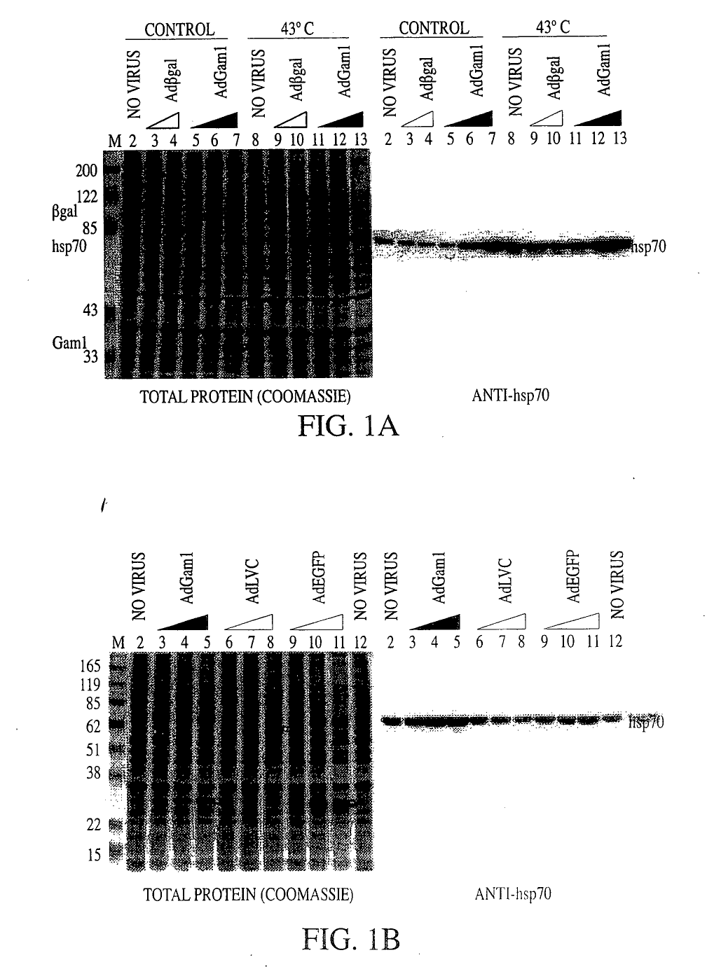

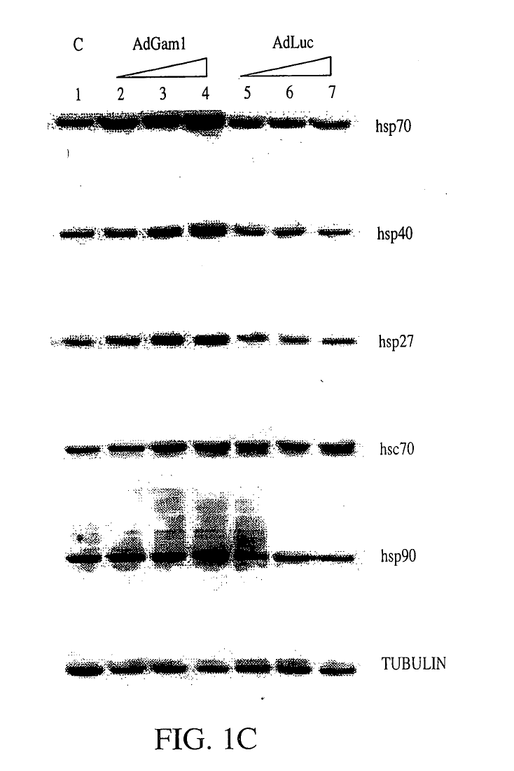

In another embodiment, assays and technique of the invention include molecular methods to identify inhibitors of heat shock protein dependent virus replication and to test the effects of candidate inhibitors on heat shock protein function and on heat shock protein interactions. In one aspect the invention discloses methods to analyze HSP levels by northern blot analyses. In another aspect the invention can be used to inhibit HSP function using antisense techniques, transfection techniques, and transgenic techniques. By way of example, molecular techniques of the invention used to assay the effects of candidate inhibitors of heat shock protein dependent virus replication are disclosed in FIGS. 1-21 and in Examples 1-3.

The invention should not be construed to be limited solely to the assays described herein, but should be construed to include other assays as well. One of skill in the art will know that other assays are available to measure protein activity and function.

Assays for Testing Inhibitors of Heat Shock Protein Dependent Virus Replication

The invention also discloses methods for measuring virus replication in the presence or absence of inhibitors of heat shock protein dependent virus replication. The methods of the invention include, but are not limited to, viral titer assays, viral focus quantitation assays, immunohistochemical analyses of viral antigens, viral antigen release assays, western blot analyses, TaqMan RT-PCR assays, particle release assays, and viral antigen capture ELISA assays.

Methods of Inhibiting or Treating Viral-Related Disease

The invention relates to inhibiting or treating viral-related diseases or disorders. Some examples of diseases which may be treated according to the methods of the invention are described herein. These viral-related diseases include, but are not limited to, acquired immune-deficiency syndrome (AIDS) and other retrovirus-induced diseases, mumps, measles, hepatitis, herpes, encephalitis, influenza, diarrhea, warts, anogenital warts, condyloma acuminata, cervical cancer and other papillomavirus related diseases, respiratory infections, conjunctivitus, hantavirus cardiopulmonary syndrome, Newcastle's disease, Kaposi's sarcoma, and Burkitt's lymphoma.

The invention should not be construed as being limited solely to these examples, as other viral-associated diseases which are at present unknown, once known, may also be treatable using the methods of the invention. In one aspect the treated disease is cancer. A cancer may belong to any of a group of cancers which have been described, as well as any other viral related cancer. Examples of such groups include, but are not limited to, leukemias and lymphomas.

The invention relates to the administration of an identified compound in a pharmaceutical composition to practice the methods of the invention, the composition comprising the compound or an appropriate derivative or fragment of the compound and a pharmaceutically-acceptable carrier. As used herein, the term “pharmaceutically-acceptable carrier” means a chemical composition with which an appropriate heat shock protein dependent virus replication inhibitor or derivative may be combined and which, following the combination, can be used to administer the appropriate inhibitor to an animal.

In one embodiment, the pharmaceutical compositions useful for practicing the invention may be administered to deliver a dose of between 1 ng/kg/day and 100 mg/kg/day.

Other pharmaceutically acceptable carriers which are useful include, hut are not limited to, glycerol, water, saline, ethanol and other pharmaceutically acceptable salt solutions such as phosphates and salts of organic acids. Examples of these and other pharmaceutically acceptable carriers are described in Remington's Pharmaceutical Sciences (1991, Mack Publication Co., New Jersey).

The pharmaceutical compositions may be prepared, packaged, or sold in the form of a sterile injectable aqueous or oily suspension or solution. This suspension or solution may be formulated according to the known art, and may comprise, in addition to the active ingredient, additional ingredients such as the dispersing agents, wetting agents, or suspending agents described herein. Such sterile injectable formulations may be prepared using a non-toxic parenterally-acceptable diluent or solvent, such as water or 1,3-butane diol, for example. Other acceptable diluents and solvents include, but are not limited to, Ringer's solution, isotonic sodium chloride solution, and fixed oils such as synthetic mono- or di-glycerides.

Pharmaceutical compositions that are useful in the methods of the invention may be administered, prepared, packaged, and/or sold in formulations suitable for oral, rectal, vaginal, parenteral, topical, pulmonary, intranasal, buccal, ophthalmic, or another route of administration. Other contemplated formulations include projected nanoparticles, liposomal preparations, resealed erythrocytes containing the active ingredient, and immunologically-based formulations.

The compositions of the invention may be administered via numerous routes, including, but not limited to, oral, rectal, vaginal, parenteral, topical, pulmonary, intranasal, buccal, or ophthalmic administration routes. The route(s) of administration will be readily apparent to the skilled artisan and will depend upon any number of factors including the type and severity of the disease being treated, the type and age of the veterinary or human patient being treated, and the like.

Pharmaceutical compositions that are useful in the methods of the invention may be administered systemically in oral solid formulations, ophthalmic, suppository, aerosol, topical or other similar formulations. In addition to the compound such as heparan sulfate, or a biological equivalent thereof, such pharmaceutical compositions may contain pharmaceutically-acceptable carriers and other ingredients known to enhance and facilitate drug administration. Other possible formulations, such as nanoparticles, liposomes, resealed erythrocytes, and immunologically based systems may also be used to administer, for example, hsp40 J domain peptides, fragments, or derivatives, and/or a nucleic acid encoding the same according to the methods of the invention. The method should not be construed to be limited to the hsp40 J domain, but should be construed to include other HSPs or proteins, fragments or derivatives thereof, as well as other types of molecules, agents, or compounds which exhibit heat shock protein dependent virus replication inhibiting activity.

Compounds which are identified using any of the methods described herein may be formulated and administered to a mammal for treatment of various viral related diseases described herein.

The invention encompasses the preparation and use of pharmaceutical compositions comprising a compound useful for treatment of various viral related diseases described herein. Such a pharmaceutical composition may consist of the active ingredient alone, in a form suitable for administration to a subject, or the pharmaceutical composition may comprise the active ingredient and one or more pharmaceutically acceptable carriers, one or more additional ingredients, or some combination of these. The active ingredient may be present in the pharmaceutical composition in the form of a physiologically acceptable ester or salt, such as in combination with a physiologically acceptable cation or anion, as is well known in the art.

As used herein, the term “pharmaceutically acceptable carrier” means a chemical composition with which the active ingredient may be combined and which, following the combination, can be used to administer the active ingredient to a subject.

As used herein, the term “physiologically acceptable” ester or salt means an ester or salt form of the active ingredient which is compatible with any other ingredients of the pharmaceutical composition, which is not deleterious to the subject to which the composition is to be administered.

The formulations of the pharmaceutical compositions described herein may be prepared by any method known or hereafter developed in the art of pharmacology. In general, such preparatory methods include the step of bringing the active ingredient into association with a carrier or one or more other accessory ingredients, and then, if necessary or desirable, shaping or packaging the product into a desired single- or multi-dose unit.

Although the descriptions of pharmaceutical compositions provided herein are principally directed to pharmaceutical compositions which are suitable for ethical administration to humans, it will be understood by the skilled artisan that such compositions are generally suitable for administration to animals of all sorts. Modification of pharmaceutical compositions suitable for administration to humans in order to render the compositions suitable for administration to various animals is well understood, and the ordinarily skilled veterinary pharmacologist can design and perform such modification with merely ordinary, if any, experimentation. Subjects to which administration of the pharmaceutical compositions of the invention is contemplated include, but are not limited to, humans and other primates, mammals including commercially relevant mammals such as cattle, pigs, horses, sheep, cats, and dogs.

Pharmaceutical compositions that are useful in the methods of the invention may be prepared, packaged, or sold in formulations suitable for oral, rectal, vaginal, parenteral, topical, pulmonary, intranasal, buccal, ophthalmic, intrathecal or another route of administration. Other contemplated formulations include projected nanoparticles, liposomal preparations, resealed erythrocytes containing the active ingredient, and immunologically-based formulations.

A pharmaceutical composition of the invention may be prepared, packaged, or sold in bulk, as a single unit dose, or as a plurality of single unit doses. As used herein, a “unit dose” is a discrete amount of the pharmaceutical composition comprising a predetermined amount of the active ingredient. The amount of the active ingredient is generally equal to the dosage of the active ingredient which would be administered to a subject or a convenient fraction of such a dosage such as, for example, one-half or one-third of such a dosage.

The relative amounts of the active ingredient, the pharmaceutically acceptable carrier, and any additional ingredients in a pharmaceutical composition of the invention will vary, depending upon the identity, size, and condition of the subject treated and further depending upon the route by which the composition is to be administered. By way of example, the composition may comprise between 0.1% and 100% (w/w) active ingredient.

In addition to the active ingredient, a pharmaceutical composition of the invention may further comprise one or more additional pharmaceutically active agents. Particularly contemplated additional agents include anti-emetics and scavengers such as cyanide and cyanate scavengers.

Controlled- or sustained-release formulations of a pharmaceutical composition of the invention may be made using conventional technology.

A formulation of a pharmaceutical composition of the invention suitable for oral administration may be prepared, packaged, or sold in the form of a discrete solid dose unit including, but not limited to, a tablet, a hard or soft capsule, a cachet, a troche, or a lozenge, each containing a predetermined amount of the active ingredient. Other formulations suitable for oral administration include, but are not limited to, a powdered or granular formulation, an aqueous or oily suspension, an aqueous or oily solution, or an emulsion.

As used herein, an “oily” liquid is one which comprises a carbon-containing liquid molecule and which exhibits a less polar character than water.

A tablet comprising the active ingredient may, for example, be made by compressing or molding the active ingredient, optionally with one or more additional ingredients. Compressed tablets may be prepared by compressing, in a suitable device, the active ingredient in a free-flowing form such as a powder or granular preparation, optionally mixed with one or more of a binder, a lubricant, an excipient, a surface active agent, and a dispersing agent. Molded tablets may be made by molding, in a suitable device, a mixture of the active ingredient, a pharmaceutically acceptable carrier, and at least sufficient liquid to moisten the mixture. Pharmaceutically acceptable excipients used in the manufacture of tablets include, but are not limited to, inert diluents, granulating and disintegrating agents, binding agents, and lubricating agents. Known dispersing agents include, but are not limited to, potato starch and sodium starch glycollate. Known surface active agents include, but are not limited to, sodium lauryl sulphate. Known diluents include, but are not limited to, calcium carbonate, sodium carbonate, lactose, microcrystalline cellulose, calcium phosphate, calcium hydrogen phosphate, and sodium phosphate. Known granulating and disintegrating agents include, but are not limited to, corn starch and alginic acid. Known binding agents include, but are not limited to, gelatin, acacia, pre-gelatinized maize starch, polyvinylpyrrolidone, and hydroxypropyl methylcellulose. Known lubricating agents include, but are not limited to, magnesium stearate, stearic acid, silica, and talc.

Tablets may be non-coated or they may be coated using known methods to achieve delayed disintegration in the gastrointestinal tract of a subject, thereby providing sustained release and absorption of the active ingredient. By way of example, a material such as glyceryl monostearate or glyceryl distearate may be used to coat tablets. Further by way of example, tablets may be coated using methods described in U.S. Pat. Nos. 4,256,108; 4,160,452; and 4,265,874 to form osmotically-controlled release tablets. Tablets may further comprise a sweetening agent, a flavoring agent, a coloring agent, a preservative, or some combination of these in order to provide for pharmaceutically elegant and palatable preparation.

Hard capsules comprising the active ingredient may be made using a physiologically degradable composition, such as gelatin. Such hard capsules comprise the active ingredient, and may further comprise additional ingredients including, for example, an inert solid diluent such as calcium carbonate, calcium phosphate, or kaolin.

Soft gelatin capsules comprising the active ingredient may be made using a physiologically degradable composition, such as gelatin. Such soft capsules comprise the active ingredient, which may be mixed with water or an oil medium such as peanut oil, liquid paraffin, or olive oil.

Liquid formulations of a pharmaceutical composition of the invention which are suitable for oral administration may be prepared, packaged, and sold either in liquid form or in the form of a dry product intended for reconstitution with water or another suitable vehicle prior to use.

Liquid suspensions may be prepared using conventional methods to achieve suspension of the active ingredient in an aqueous or oily vehicle. Aqueous vehicles include, for example, water and isotonic saline. Oily vehicles include, for example, almond oil, oily esters, ethyl alcohol, vegetable oils such as arachis, olive, sesame, or coconut oil, fractionated vegetable oils, and mineral oils such as liquid paraffin. Liquid suspensions may further comprise one or more additional ingredients including, but not limited to, suspending agents, dispersing or wetting agents, emulsifying agents, demulcents, preservatives, buffers, salts, flavorings, coloring agents, and sweetening agents. Oily suspensions may further comprise a thickening agent. Known suspending agents include, but are not limited to, sorbitol syrup, hydrogenated edible fats, sodium alginate, polyvinylpyrrolidone, gum tragacanth, gum acacia, and cellulose derivatives such as sodium carboxymethylcellulose, methylcellulose, hydroxypropylmethylcellulose. Known dispersing or wetting agents include, but are not limited to, naturally-occurring phosphatides such as lecithin, condensation products of an alkylene oxide with a fatty acid, with a long chain aliphatic alcohol, with a partial ester derived from a fatty acid and a hexitol, or with a partial ester derived from a fatty acid and a hexitol anhydride (e.g., polyoxyethylene stearate, heptadecaethyleneoxycetanol, polyoxyethylene sorbitol monooleate, and polyoxyethylene sorbitan monooleate, respectively). Known emulsifying agents include, but are not limited to, lecithin and acacia. Known preservatives include, but are not limited to, methyl, ethyl, or n-propyl-para-hydroxybenzoates, ascorbic acid, and sorbic acid. Known sweetening agents include, for example, glycerol, propylene glycol, sorbitol, sucrose, and saccharin. Known thickening agents for oily suspensions include, for example, beeswax, hard paraffin, and cetyl alcohol.

Liquid solutions of the active ingredient in aqueous or oily solvents may be prepared in substantially the same manner as liquid suspensions, the primary difference being that the active ingredient is dissolved, rather than suspended in the solvent. Liquid solutions of the pharmaceutical composition of the invention may comprise each of the components described with regard to liquid suspensions, it being understood that suspending agents will not necessarily aid dissolution of the active ingredient in the solvent. Aqueous solvents include, for example, water and isotonic saline. Oily solvents include, for example, almond oil, oily esters, ethyl alcohol, vegetable oils such as arachis, olive, sesame, or coconut oil, fractionated vegetable oils, and mineral oils such as liquid paraffin.

Powdered and granular formulations of a pharmaceutical preparation of the invention may be prepared using known methods. Such formulations may be administered directly to a subject, used, for example, to form tablets, to fill capsules, or to prepare an aqueous or oily suspension or solution by addition of an aqueous or oily vehicle thereto. Each of these formulations may further comprise one or more of dispersing or wetting agent, a suspending agent, and a preservative. Additional excipients, such as fillers and sweetening, flavoring, or coloring agents, may also be included in these formulations.

A pharmaceutical composition of the invention may also be prepared, packaged, or sold in the form of oil-in-water emulsion or a water-in-oil emulsion. The oily phase may be a vegetable oil such as olive or arachis oil, a mineral oil such as liquid paraffin, or a combination of these. Such compositions may further comprise one or more emulsifying agents such as naturally occurring gums such as gum acacia or gum tragacanth, naturally-occurring phosphatides such as soybean or lecithin phosphatide, esters or partial esters derived from combinations of fatty acids and hexitol anhydrides such as sorbitan monooleate, and condensation products of such partial esters with ethylene oxide such as polyoxyethylene sorbitan monooleate. These emulsions may also contain additional ingredients including, for example, sweetening or flavoring agents.

A pharmaceutical composition of the invention may be prepared, packaged, or sold in a formulation suitable for rectal administration. Such a composition may be in the form of, for example, a suppository, a retention enema preparation, and a solution for rectal or colonic irrigation.

Suppository formulations may be made by combining the active ingredient with a non-irritating pharmaceutically acceptable excipient which is solid at ordinary room temperature (i.e., about 20° C.) and which is liquid at the rectal temperature of the subject (i.e., about 37° C. in a healthy human). Suitable pharmaceutically acceptable excipients include, but are not limited to, cocoa butter, polyethylene glycols, and various glycerides. Suppository formulations may further comprise various additional ingredients including, but not limited to, antioxidants and preservatives.

Retention enema preparations or solutions for rectal or colonic irrigation may be made by combining the active ingredient with a pharmaceutically acceptable liquid carrier. As is well known in the art, enema preparations may be administered using, and may be packaged within, a delivery device adapted to the rectal anatomy of the subject. Enema preparations may further comprise various additional ingredients including, but not limited to, antioxidants and preservatives.

A pharmaceutical composition of the invention may be prepared, packaged, or sold in a formulation suitable for vaginal administration. Such a composition may be in the form of, for example, a suppository, an impregnated or coated vaginally-insertable material such as a tampon, a douche preparation, or gel or cream or a solution for vaginal irrigation.

Methods for impregnating or coating a material with a chemical composition are known in the art, and include, but are not limited to methods of depositing or binding a chemical composition onto a surface, methods of incorporating a chemical composition into the structure of a material during the synthesis of the material (i.e., such as with a physiologically degradable material), and methods of absorbing an aqueous or oily solution or suspension into an absorbent material, with or without subsequent drying.

Douche preparations or solutions for vaginal irrigation may be made by combining the active ingredient with a pharmaceutically acceptable liquid carrier. As is well known in the art, douche preparations may be administered using, and may be packaged within, a delivery device adapted to the vaginal anatomy of the subject. Douche preparations may further comprise various additional ingredients including, but not limited to, antioxidants, antibiotics, antifungal agents, and preservatives.

As used herein, “parenteral administration” of a pharmaceutical composition includes any route of administration characterized by physical breaching of a tissue of a subject and administration of the pharmaceutical composition through the breach in the tissue. Parenteral administration thus includes, but is not limited to, administration of a pharmaceutical composition by injection of the composition, by application of the composition through a surgical incision, by application of the composition through a tissue-penetrating non-surgical wound, and the like. In particular, parenteral administration is contemplated to include, but is not limited to, subcutaneous, intraperitoneal, intramuscular, intrasternal injection, and kidney dialytic infusion techniques.

Formulations of a pharmaceutical composition suitable for parenteral administration comprise the active ingredient combined with a pharmaceutically acceptable carrier, such as sterile water or sterile isotonic saline. Such formulations may be prepared, packaged, or sold in a form suitable for bolus administration or for continuous administration. Injectable formulations may be prepared, packaged, or sold in unit dosage form, such as in ampules or in multi-dose containers containing a preservative. Formulations for parenteral administration include, but are not limited to, suspensions, solutions, emulsions in oily or aqueous vehicles, pastes, and implantable sustained-release or biodegradable formulations. Such formulations may further comprise one or more additional ingredients including, but not limited to, suspending, stabilizing, or dispersing agents. In one embodiment of a formulation for parenteral administration, the active ingredient is provided in dry (i.e., powder or granular) form for reconstitution with a suitable vehicle (e.g., sterile pyrogen-free water) prior to parenteral administration of the reconstituted composition.

The pharmaceutical compositions may be prepared, packaged, or sold in the form of a sterile injectable aqueous or oily suspension or solution. This suspension or solution may be formulated according to the known art, and may comprise, in addition to the active ingredient, additional ingredients such as the dispersing agents, wetting agents, or suspending agents described herein. Such sterile injectable formulations may be prepared using a non-toxic parenterally-acceptable diluent or solvent, such as water or 1,3-butane diol, for example. Other acceptable diluents and solvents include, but are not limited to, Ringer's solution, isotonic sodium chloride solution, and fixed oils such as synthetic mono- or di-glycerides. Other parentally-administrable formulations which are useful include those which comprise the active ingredient in microcrystalline form, in a liposomal preparation, or as a component of a biodegradable polymer system. Compositions for sustained release or implantation may comprise pharmaceutically acceptable polymeric or hydrophobic materials such as an emulsion, an ion exchange resin, a sparingly soluble polymer, or a sparingly soluble salt.

Formulations suitable for topical administration include, but are not limited to, liquid or semi-liquid preparations such as liniments, lotions, oil-in-water or water-in-oil emulsions such as creams, ointments or pastes, and solutions or suspensions. Topically-administrable formulations may, for example, comprise from about 1% to about 10% (w/w) active ingredient, although the concentration of the active ingredient may be as high as the solubility limit of the active ingredient in the solvent. Formulations for topical administration may further comprise one or more of the additional ingredients described herein.

A pharmaceutical composition of the invention may be prepared, packaged, or sold in a formulation suitable for pulmonary administration via the buccal cavity. Such a formulation may comprise dry particles which comprise the active ingredient and which have a diameter in the range from about 0.5 to about 7 nanometers, and preferably from about 1 to about 6 nanometers. Such compositions are conveniently in the form of dry powders for administration using a device comprising a dry powder reservoir to which a stream of propellant may be directed to disperse the powder or using a self-propelling solvent/powder-dispensing container such as a device comprising the active ingredient dissolved or suspended in a low-boiling propellant in a sealed container. Preferably, such powders comprise particles wherein at least 98% of the particles by weight have a diameter greater than 0.5 nanometers and at least 95% of the particles by number have a diameter less than 7 nanometers. More preferably, at least 95% of the particles by weight have a diameter greater than 1 nanometer and at least 90% of the particles by number have a diameter less than 6 nanometers. Dry powder compositions preferably include a solid fine powder diluent such as sugar and are conveniently provided in a unit dose form.

Low boiling propellants generally include liquid propellants having a boiling point of below 65° F. at atmospheric pressure. Generally the propellant may constitute 50 to 99.9% (w/w) of the composition, and the active ingredient may constitute 0.1 to 20% (w/v) of the composition. The propellant may further comprise additional ingredients such as a liquid non-ionic or solid anionic surfactant or a solid diluent (preferably having a particle size of the same order as particles comprising the active ingredient).

Pharmaceutical compositions of the invention formulated for pulmonary delivery may also provide the active ingredient in the form of droplets of a solution or suspension. Such formulations may be prepared, packaged, or sold as aqueous or dilute alcoholic solutions or suspensions, optionally sterile, comprising the active ingredient, and may conveniently be administered using any nebulization or atomization device. Such formulations may further comprise one or more additional ingredients including, but not limited to, a flavoring agent such as saccharin sodium, a volatile oil, a buffering agent, a surface active agent, or a preservative such a methylhydroxybenzoate. The droplets provided by this route of administration preferably have an average diameter in the range from about 0.1 to about 200 nanometers.

The formulations described herein as being useful for pulmonary delivery are also useful for intranasal delivery of a pharmaceutical composition of the invention.

Another formulation suitable for intranasal administration is a coarse powder comprising the active ingredient and having an average particle from about 0.2 to 500 micrometers. Such a formulation is administered in the manner in which snuff is taken, i.e., by rapid inhalation through the nasal passage from a container of the powder held close to the nares.

Formulations suitable for nasal administration may, for example, comprise from about as little as 0.1% (w/w) and as much as 100% (w/w) of the active ingredient, and may further comprise one or more of the additional ingredients described herein.

A pharmaceutical composition of the invention may be prepared, packaged, or sold in a formulation suitable for buccal administration. Such formulations may, for example, be in the form of tablets or lozenges made using conventional methods, and may, for example, 0.1 to 20% (w/w) active ingredient, the balance comprising an orally dissolvable or degradable composition and, optionally, one or more of the additional ingredients described herein. Alternately, formulations suitable for buccal administration may comprise a powder or an aerosolized or atomized solution or suspension comprising the active ingredient. Such powdered, aerosolized, or aerosolized formulations, when dispersed, preferably have an average particle or droplet size in the range from about 0.1 to about 200 nanometers, and may further comprise one or more of the additional ingredients described herein.

A pharmaceutical composition of the invention may be prepared, packaged, or sold in a formulation suitable for ophthalmic administration. Such formulations may, for example, be in the form of eye drops including, for example, a 0.1-1.0%, (w/w) solution or suspension of the active ingredient in an aqueous or oily liquid carrier. Such drops may further comprise buffering agents, salts, or one or more other of the additional ingredients described herein. Other ophthalmically-adminiistrable formulations which are useful include those which comprise the active ingredient in microcrystalline form or in a liposomal preparation.

As used herein, “additional ingredients” include, but are not limited to, one or more of the following: excipients; surface active agents; dispersing agents; inert diluents; granulating and disintegrating agents; binding agents; lubricating agents; sweetening agents; flavoring agents; coloring agents; preservatives; physiologically degradable compositions such as gelatin; aqueous vehicles and solvents; oily vehicles and solvents; suspending agents; dispersing or wetting agents; emulsifying agents, demulcents; buffers; salts; thickening agents; fillers; emulsifying agents; antioxidants; antibiotics; antifungal agents; stabilizing agents; and pharmaceutically acceptable polymeric or hydrophobic materials. Other “additional ingredients” which may be included in the pharmaceutical compositions of the invention are known in the art and described, for example in Genaro, ed. (1985, Remington's Pharmaceutical Sciences, Mack Publishing Co., Easton, Pa.), which is incorporated herein by reference.

Typically, dosages of the compound of the invention which may be administered to an animal, preferably a human, will vary depending upon any number of factors, including but not limited to, the type of animal and type of disease state being treated, the age of the animal and the route of administration.

The compound can be administered to an animal as frequently as several times daily, or it may be administered less frequently, such as once a day, once a week, once every two weeks, once a month, or even lees frequently, such as once every several months or even once a year or less. The frequency of the dose will be readily apparent to the skilled artisan and will depend upon any number of factors, such as, but not limited to, the type and severity of the disease being treated, the type and age of the animal, etc.

As used herein, “alleviating a viral disease or disorder symptom” means reducing the severity of the symptom.

As used herein, “treating a viral disease or disorder” means reducing the frequency with which a symptom of the viral disease or disorder is experienced by a patient. Viral disease or disorder is used interchangeably herein with virus-related disease or disorder and viral-related disease or disorder.

A “prophylactic” treatment is a treatment administered to a subject who does not exhibit signs of a disease or exhibits only early signs of the disease for the purpose of decreasing the risk of developing pathology associated with the disease.

A “therapeutic” treatment is a treatment administered to a subject who exhibits signs of pathology for the purpose of diminishing or eliminating those signs.

A “therapeutically effective amount” of a compound is that amount of compound which is sufficient to provide a beneficial effect to the subject to which the compound is administered.

A “disease” is a state of health of an animal wherein the animal cannot maintain homeostasis, and wherein if the disease is not ameliorated then the animal's health continues to deteriorate.

In contrast, a “disorder” in an animal is a state of health in which the animal is able to maintain homeostasis, but in which the animal's state of health is less favorable than it would be in the absence of the disorder. Left untreated, a disorder does not necessarily cause a further decrease in the animal's state of health.

A disease or disorder is “alleviated” if the severity of a symptom of the disease or disorder, the frequency with which such a symptom is experienced by a patient, or both, are reduced.

It will be recognized by one of skill in the art that the various embodiments of the invention as described above relating to methods of treating viral-related diseases encompasses adenovirus, hantavirus and HIV-1, as well as viruses not described herein. Thus, it should not be construed that embodiments described herein for adenovirus, hantavirus, and HIV-1, do not apply to other viruses.

Kits for Inhibiting Virus Replication

The method of the invention includes a kit comprising an inhibitor identified in the invention and an instructional material which describes administering the inhibitor or a composition comprising the inhibitor to a cell or an animal. This should be construed to include other embodiments of kits that are known to those skilled in the art, such as a kit comprising a (preferably sterile) solvent suitable for dissolving or suspending the composition of the invention prior to administering the compound to a cell or an animal. Preferably the animal is a human.

As used herein, an “instructional material” includes a publication, a recording, a diagram, or any other medium of expression which can be used to communicate the usefulness of the composition of the invention for its designated use. The instructional material of the kit of the invention may, for example, be affixed to a container which contains the composition or be shipped together with a container which contains the composition. Alternatively, the instructional material may be shipped separately from the container with the intention that the instructional material and the composition be used cooperatively by the recipient.

Definitions

The articles “a” and “an” are used herein to refer to one or to more than one (i.e., to at least one) of the grammatical object of the article. By way of example, “an element” means one element or more than one element.

As used herein, amino acids are represented by the full name thereof, by the three letter code corresponding thereto, or by the one-letter code corresponding thereto, as indicated in the following table:

| Full Name | Three-Letter Code | One-Letter Code |
|---|---|---|
| Aspartic Acid | Asp | D |
| Glutamic Acid | Glu | E |
| Lysine | Lys | K |
| Arginine | Arg | R |
| Histidine | His | H |
| Tyrosine | Tyr | Y |
| Cysteine | Cys | C |
| Asparagine | Asn | N |
| Glutamine | Gln | Q |
| Serine | Ser | S |
| Threonine | Thr | T |
| Glycine | Gly | G |
| Alanine | Ala | A |
| Valine | Val | V |
| Leucine | Leu | L |
| Isoleucine | Ile | I |
| Methionine | Met | M |
| Proline | Pro | P |
| Phenylalanine | Phe | F |
| Tryptophan | Trp | W |

The term “antibody,” as used herein, refers to an immunoglobulin molecule which is able to specifically bind to a specific epitope on an antigen. Antibodies can be intact immunoglobulins derived from natural sources or from recombinant sources and can be immunoreactive portions of intact immunoglobulins. Antibodies are typically tetramers of immunoglobulin molecules. The antibodies in the present invention may exist in a variety of forms including, for example, polyclonal antibodies, monoclonal antibodies, Fv, Fab and F(ab)₂, as well as single chain antibodies and humanized antibodies.

As used herein, the term “antisense oligonucleotide” or antisense nucleic acid means a nucleic acid polymer, at least a portion of which is complementary to a nucleic acid which is present in a normal cell or in an affected cell. “Antisense” refers particularly to the nucleic acid sequence of the non-coding strand of a double stranded DNA molecule encoding a protein, or to a sequence which is substantially homologous to the non-coding strand. As defined herein, an antisense sequence is complementary to the sequence of a double stranded DNA molecule encoding a protein. It is not necessary that the antisense sequence be complementary solely to the coding portion of the coding strand of the DNA molecule. The antisense sequence may be complementary to regulatory sequences specified on the coding strand of a DNA molecule encoding a protein, which regulatory sequences control expression of the coding sequences. The antisense oligonucleotides of the invention include, but are not limited to, phosphorothioate oligonucleotides and other modifications of oligonucleotides.

“Antiviral agent,” as used herein means a composition of matter which, when delivered to a cell, is capable of preventing replication of a virus in the cell, preventing infection of the cell by a virus, or reversing a physiological effect of infection of the cell by a virus. Antiviral agents are well known and described in the literature. By way of example, AZT (zidovudine, Retrovir® Glaxo Wellcome Inc., Research Triangle Park, N.C.) is an antiviral agent which is thought to prevent replication of HIV in human cells.

A “coding region” of a gene consists of the nucleotide residues of the coding strand of the gene and the nucleotides of the non-coding strand of the gene which are homologous with or complementary to, respectively, the coding region of an mRNA molecule which is produced by transcription of the gene.

“Complementary” as used herein refers to the broad concept of subunit sequence complementarity between two nucleic acids, e.g., two DNA molecules. When a nucleotide position in both of the molecules is occupied by nucleotides normally capable of base pairing with each other, then the nucleic acids are considered to be complementary to each other at this position. Thus, two nucleic acids are complementary to each other when a substantial number (at least 50%) of corresponding positions in each of the molecules are occupied by nucleotides which normally base pair with each other (e.g., A:T and G:C nucleotide pairs). Thus, it is known that an adenine residue of a first nucleic acid region is capable of forming specific hydrogen bonds (“base pairing”) with a residue of a second nucleic acid region which is antiparallel to the first region if the residue is thymine or uracil. Similarly, it is known that a cytosine residue of a first nucleic acid strand is capable of base pairing with a residue of a second nucleic acid strand which is antiparallel to the first strand if the residue is guanine. A first region of a nucleic acid is complementary to a second region of the same or a different nucleic acid if, when the two regions are arranged in an antiparallel fashion, at least one nucleotide residue of the first region is capable of base pairing with a residue of the second region. Preferably, the first region comprises a first portion and the second region comprises a second portion, whereby, when the first and second portions are arranged in an antiparallel fashion, at least about 50%, and preferably at least about 75%, at least about 90%, or at least about 95% of the nucleotide residues of the first portion are capable of base pairing with nucleotide residues in the second portion. More preferably, all nucleotide residues of the first portion are capable of base pairing with nucleotide residues in the second portion.

A “compound,” as used herein, refers to any type of substance or agent that is commonly considered a drug, or a candidate for use as a drug, as well as combinations and mixtures of the above.

“Cytokine,” as used herein, refers to intercellular signaling molecules, the best known of which are involved in the regulation of mammalian somatic cells. A number of families of cytokines, both growth promoting and growth inhibitory in their effects, have been characterized including, for example, interleukins, interferons, and transforming growth factors. A number of other cytokines are known to those of skill in the art. The sources, characteristics, targets and effector activities of these cytokines have been described.

“Encoding” refers to the inherent property of specific sequences of nucleotides in a polynucleotide, such as a gene, a cDNA, or an mRNA, to serve as templates for synthesis of other polymers and macromolecules in biological processes having either a defined sequence of nucleotides (i.e., rRNA, tRNA and mRNA) or a defined sequence of amino acids and the biological properties resulting therefrom. Thus, a gene encodes a protein if transcription and translation of mRNA corresponding to that gene produces the protein in a cell or other biological system. Both the coding strand, the nucleotide sequence of which is identical to the mRNA sequence and is usually provided in sequence listings, and the non-coding strand, used as the template for transcription of a gene or cDNA, can be referred to as encoding the protein or other product of that gene or cDNA.

Unless otherwise specified, a “nucleotide sequence encoding an amino acid sequence” includes all nucleotide sequences that are degenerate versions of each other and that encode the same amino acid sequence. Nucleotide sequences that encode proteins and RNA may include introns.

As used herein, the term “fragment” as applied to a nucleic acid, may ordinarily be at least about 20 nucleotides in length, typically, at least about 50 nucleotides, more typically, from about 50 to about 100 nucleotides, preferably, at least about 100 to about 200 nucleotides, even more preferably, at least about 200 nucleotides to about 300 nucleotides, yet even more preferably, at least about 300 to about 350, even more preferably, at least about 350 nucleotides to about 500 nucleotides, yet even more preferably, at least about 500 to about 600, even more preferably, at least about 600 nucleotides to about 620 nucleotides, yet even more preferably, at least about 620 to about 650, and most preferably, the nucleic acid fragment will be greater than about 650 nucleotides in length.

“Heat shock protein inhibitor,” as used herein, refers to any agent, the application of which results in the inhibition of a heat shock protein function or heat shock protein pathway function.

By “heat shock protein interaction” is meant a functional relationship of one HSP with another HSP, or of an HSP with a component of its heat shock response pathway, and includes, but is not limited to, binding of one type of HSP to another type of HSP, such as hsp40 with hsp70. The interaction results in a modification of an activity or the gain or loss of activity. For example, it is known that hsp40 is a cofactor of hsp70 and that the hsp40 J domain appears to be required for the protein-protein interaction. The resulting interaction enhances hsp70 function.

As used herein, “heat shock response” refers to a stress response by a cell to various perturbations, including viruses and heat shock. The response includes, but is not limited to elevation and relocalization of a variety of HSPs, as well as interactions of HSPs.

“Homologous” as used herein, refers to the subunit sequence similarity between two polymeric molecules, e.g., between two nucleic acid molecules, e.g., two DNA molecules or two RNA molecules, or between two polypeptide molecules. When a subunit position in both of the two molecules is occupied by the same monomeric subunit, e.g., if a position in each of two DNA molecules is occupied by adenine, then they are homologous at that position. The homology between two sequences is a direct function of the number of matching or homologous positions, e.g., if half (e.g., five positions in a polymer ten subunits in length) of the positions in two compound sequences are homologous then the two sequences are 50% homologous, if 90% of the positions, e.g., 9 of 10, are matched or homologous, the two sequences share 90% homology. By way of example, the DNA sequences 3′ATTGCC5′ and 3′TATGGC share 50% homology.

As used herein, “homology” is used synonymously with “identity.”

The determination of percent identity between two nucleotide or amino acid sequences can be accomplished using a mathematical algorithm. For example, a mathematical algorithm useful for comparing two sequences is the algorithm of Karlin and Altschul (1990, Proc. Natl. Acad. Sci. USA 87:2264-2268), modified as in Karlin and Altschul (1993, Proc. Natl. Acad. Sci. USA 90:5873-5877). This algorithm is incorporated into the NBLAST and XBLAST programs of Altschul, et al. (1990, J. Mol. Biol. 215:403-410), and can be accessed, for example at the National Center for Biotechnology Information (NCBI) world wide web site having the universal resource locator “http://www.ncbi.nlm.nih.gov/BLAST/”. BLAST nucleotide searches can be performed with the NBLAST program (designated “blastn” at the NCBI web site), using the following parameters: gap penalty=5; gap extension penalty=2; mismatch penalty=3; match reward=1; expectation value 10.0; and word size=11 to obtain nucleotide sequences homologous to a nucleic acid described herein. BLAST protein searches can be performed with the XBLAST program (designated “blastn” at the NCBI web site) or the NCBI “blastp” program, using the following parameters: expectation value 10.0, BLOSUM62 scoring matrix to obtain amino acid sequences homologous to a protein molecule described herein. To obtain gapped alignments for comparison purposes, Gapped BLAST can be utilized as described in Altschul et al. (1997, Nucleic Acids Res. 25:3389-3402). Alternatively, PSI-Blast or PHI-Blast can be used to perform an iterated search which detects distant relationships between molecules (Id.) and relationships between molecules which share a common pattern. When utilizing BLAST, Gapped BLAST, PSI-Blast, and PHI-Blast programs, the default parameters of the respective programs (e.g., XBLAST and NBLAST) can be used. See http://www.ncbi.nlm.nih.gov.

The percent identity between two sequences can be determined using techniques similar to those described above, with or without allowing gaps. In calculating percent identity, typically exact matches are counted.

As used herein, an “instructional material” includes a publication, a recording, a diagram, or any other medium of expression which can be used to communicate the usefulness of the peptide of the invention in the kit for effecting alleviation of the various diseases or disorders recited herein. Optionally, or alternately, the instructional material may describe one or more methods of alleviating the diseases or disorders in a cell or a tissue of a mammal. The instructional material of the kit of the invention may, for example, be affixed to a container which contains the identified compound invention or be shipped together with a container which contains the identified compound. Alternatively, the instructional material may be shipped separately from the container with the intention that the instructional material and the compound be used cooperatively by the recipient.

An “isolated nucleic acid” refers to a nucleic acid segment or fragment which has been separated from sequences which flank it in a naturally occurring state, e.g., a DNA fragment which has been removed from the sequences which are normally adjacent to the fragment, e.g., the sequences adjacent to the fragment in a genome in which it naturally occurs. The term also applies to nucleic acids which have been substantially purified from other components which naturally accompany the nucleic acid, e.g., RNA or DNA or proteins, which naturally accompany it in the cell. The term therefore includes, for example, a recombinant DNA which is incorporated into a vector, into an autonomously replicating plasmid or virus, or into the genomic DNA of a prokaryote or eukaryote, or which exists as a separate molecule (e.g, as a cDNA or a genomic or cDNA fragment produced by PCR or restriction enzyme digestion) independent of other sequences. It also includes a recombinant DNA which is part of a hybrid gene encoding additional polypeptide sequence.

A “ligand” is a compound that specifically binds to a target receptor.

A “receptor” is a compound that specifically binds to a ligand.

A ligand or a receptor (e.g., an antibody) “specifically binds to” or “is specifically immunoreactive with” a compound when the ligand or receptor functions in a binding reaction which is determinative of the presence of the compound in a sample of heterogeneous compounds. Thus, under designated assay (e.g., immunoassay) conditions, the ligand or receptor binds preferentially to a particular compound and does not bind in a significant amount to other compounds present in the sample. For example, a polynucleotide specifically binds under hybridization conditions to a compound polynucleotide comprising a complementary sequence; an antibody specifically binds under immunoassay conditions to an antigen bearing an epitope against which the antibody was raised. A variety of immunoassay formats may be used to select antibodies specifically immunoreactive with a particular protein. For example, solid-phase ELISA immunoassays are routinely used to select monoclonal antibodies specifically immunoreactive with a protein. See Harlow and Lane (1988, Antibodies, A Laboratory Manual, Cold Spring Harbor Publications, New York) for a description of immunoassay formats and conditions that can be used to determine specific immunoreactivity.

“Linker” refers to a molecule that joins two other molecules, either covalently, or through ionic, van der Waals or hydrogen bonds, e.g., a nucleic acid molecule that hybridizes to one complementary sequence at the 5′ end and to another complementary sequence at the 3′ end, thus joining two non-complementary sequences.

The term “nucleic acid” typically refers to large polynucleotides.

The term “oligonucleotide” typically refers to short polynucleotides, generally, no greater than about 50 nucleotides. It will be understood that when a nucleotide sequence is represented by a DNA sequence (i.e., A, T, G, C), this also includes an RNA sequence (i.e., A, U, G, C) in which “U” replaces “T.”

The term “nucleic acid construct,” as used herein, encompasses DNA and RNA sequences encoding the particular gene or gene fragment desired, whether obtained by genomic or synthetic methods.

By describing two polynucleotides as “operably linked” is meant that a single-stranded or double-stranded nucleic acid moiety comprises the two polynucleotides arranged within the nucleic acid moiety in such a manner that at least one of the two polynucleotides is able to exert a physiological effect by which it is characterized upon the other. By way of example, a promoter operably linked to the coding region of a gene is able to promote transcription of the coding region.

A “polynucleotide” means a single strand or parallel and anti-parallel strands of a nucleic acid. Thus, a polynucleotide may be either a single-stranded or a double-stranded nucleic acid.

“Polypeptide” refers to a polymer composed of amino acid residues, related naturally occurring structural variants, and synthetic non-naturally occurring analogs thereof linked via peptide bonds, related naturally occurring structural variants, and synthetic non-naturally occurring analogs thereof.

“Synthetic peptides or polypeptides” means a non-naturally occurring peptide or polypeptide. Synthetic peptides or polypeptides can be synthesized, for example, using an automated polypeptide synthesizer. Various solid phase peptide synthesis methods are known to those of skill in the art.

“Primer” refers to a polynucleotide that is capable of specifically hybridizing to a designated polynucleotide template and providing a point of initiation for synthesis of a complementary polynucleotide. Such synthesis occurs when the polynucleotide primer is placed under conditions in which synthesis is induced, i.e., in the presence of nucleotides, a complementary polynucleotide template, and an agent for polymerization such as DNA polymerase. A primer is typically single-stranded, but may be double-stranded. Primers are typically deoxyribonucleic acids, but a wide variety of synthetic and naturally occurring primers are useful for many applications. A primer is complementary to the template to which it is designed to hybridize to serve as a site for the initiation of synthesis, but need not reflect the exact sequence of the template. In such a case, specific hybridization of the primer to the template depends on the stringency of the hybridization conditions. Primers can be labeled with, e.g., chromogenic, radioactive, or fluorescent moieties and used as detectable moieties.

As used herein, the term “promoter/regulatory sequence” means a nucleic acid sequence which is required for expression of a gene product operably linked to the promoter/regulator sequence. In some instances, this sequence may be the core promoter sequence and in other instances, this sequence may also include an enhancer sequence and other regulatory elements which are required for expression of the gene product. The promoter/regulatory sequence may, for example, be one which expresses the gene product in a tissue specific manner.

A “constitutive” promoter is a promoter which drives expression of a gene to which it is operably linked, in a constant manner in a cell. By way of example, promoters which drive expression of cellular housekeeping genes are considered to be constitutive promoters.

An “inducible” promoter is a nucleotide sequence which, when operably linked with a polynucleotide which encodes or specifies a gene product, causes the gene product to be produced in a living cell substantially only when an inducer which corresponds to the promoter is present in the cell.

A “tissue-specific” promoter is a nucleotide sequence which, when operably linked with a polynucleotide which encodes or specifies a gene product, causes the gene product to be produced in a living cell substantially only if the cell is a cell of the tissue type corresponding to the promoter.

The term “protein” typically refers to large polypeptides.

The term “peptide” typically refers to short polypeptides.

“Recombinant polynucleotide” refers to a polynucleotide having sequences that are not naturally joined together. An amplified or assembled recombinant polynucleotide may be included in a suitable vector, and the vector can be used to transform a suitable host cell.

A recombinant polynucleotide may serve a non-coding function (e.g., promoter, origin of replication, ribosome-binding site, etc.) as well.

A host cell that comprises a recombinant polynucleotide is referred to as a “recombinant host cell.” A gene which is expressed in a recombinant host cell wherein the gene comprises a recombinant polynucleotide, produces a “recombinant polypeptide.”

A “recombinant polypeptide” is one which is produced upon expression of a recombinant polynucleotide.

“Polypeptide” refers to a polymer composed of amino acid residues, related naturally occurring structural variants, and synthetic non-naturally occurring analogs thereof linked via peptide bonds, related naturally occurring structural variants, and synthetic non-naturally occurring analogs thereof. Synthetic polypeptides can be synthesized, for example, using an automated polypeptide synthesizer.

The term “protein” typically refers to large polypeptides.

The term “peptide” typically refers to short polypeptides.

Conventional notation is used herein to portray polypeptide sequences: the left-hand end of a polypeptide sequence is the amino-terminus; the right-hand end of a polypeptide sequence is the carboxyl-terminus.

A “receptor” is a compound that specifically binds to a ligand.

A “ligand” is a compound that specifically binds to a target receptor.

As used herein, the term “reporter gene” means a gene, the expression of which can be detected using a known method. By way of example, the Escherichia coli lacZ gene may be used as a reporter gene in a medium because expression of the lacZ gene can be detected using known methods by adding the chromogenic substrate o-nitrophenyl- -galactoside to the medium (Gerhardt et al., eds., 1994, Methods for General and Molecular Bacteriology, American Society for Microbiology, Washington, D.C., p. 574).

A “recombinant cell” is a cell that comprises a transgene. Such a cell may be a eukaryotic or a prokaryotic cell. Also, the transgenic cell encompasses, but is not limited to, an embryonic stem cell comprising the transgene, a cell obtained from a chimeric mammal derived from a transgenic embryonic stem cell where the cell comprises the transgene, a cell obtained from a transgenic mammal, or fetal or placental tissue thereof, and a prokaryotic cell comprising the transgene.

A “subject” of diagnosis or treatment is a mammal, including a human. Non-human animals subject to diagnosis or treatment include, for example, livestock and pets.

By “tag” polypeptide is meant any protein which, when linked by a peptide bond to a protein of interest, may be used to localize the protein, to purify it from a cell extract, to immobilize it for use in binding assays, or to otherwise study its biological properties and/or function.

As used herein, the term “transgene” means an exogenous nucleic acid sequence which exogenous nucleic acid is encoded by a transgenic cell or mammal.

As used herein, the term “transgenic mammal” means a manual, the germ cells of which comprise an exogenous nucleic acid.

The term to “treat,” as used herein, means reducing the frequency with which symptoms are experienced by a patient or subject or administering an agent or compound to reduce the frequency with which symptoms are experienced.

A “vector” is a composition of matter which comprises an isolated nucleic acid and which can be used to deliver the isolated nucleic acid to the interior of a cell. Numerous vectors are known in the art including, but not limited to, linear polynucleotides, polynucleotides associated with ionic or amphiplilic compounds, plasmids, and viruses. Thus, the term “vector” includes an autonomously replicating plasmid or a virus. The term should also be construed to include non-plasmid and non-viral compounds which facilitate transfer or delivery of nucleic acid to cells, such as, for example, polylysine compounds, liposomes, and the like. Examples of viral vectors include, but are not limited to, adenoviral vectors, adeno-associated virus vectors, retroviral vectors, recombinant viral vectors, and the like. Examples of non-viral vectors include, but are not limited to, liposomes, polyamine derivatives of DNA and the like.

“Expression vector” refers to a vector comprising a recombinant polynucleotide comprising expression control sequences operatively linked to a nucleotide sequence to be expressed. An expression vector comprises sufficient cis-acting elements for expression; other elements for expression can be supplied by the host cell or in an in vitro expression system. Expression vectors include all those known in the art, such as cosmids, plasmids (e.g., naked or contained in liposomes) and viruses that incorporate the recombinant polynucleotide.

A “virus replication-inhibiting amount” as used herein means the amount of an inhibitor necessary to detectably inhibit or reduce virus replication in a cell or an animal, compared with the level of virus replication when the inhibitor is not present. It also means the amount of inhibitor required to reduce virus replication when the inhibitor is added to an animal or cell in which virus replication has already begun, compared to the amount of virus replication in the absence of the inhibitor.

### EXPERIMENTAL EXAMPLES

The invention is now described with reference to the following Examples. These Examples are provided for the purpose of illustration only and the invention should in no way be construed as being limited to these Examples, but rather should be construed to encompass any and all variations which become evident as a result of the teaching provided herein.

### Example 1

### CELO Adenovirus

Gam1, a histone deacetylase inhibitor encoded by the avian adenovirus CELO, was identified in an anti-apoptosis screen (Chiocca et al., 1997, J. Virol. 71:3168-3177). The nuclear location of Gain I suggested that the protein might influence the expression of genes whose products modulate apoptosis. Among these, hsp70 expression has been correlated with increased cell survival under stress (Gahai et al., 1997, J. Biol. Chem. 272:18033-18037; Mosser et al., 1997, Mol. Cell. Biol. 17:5317-5327). In the results described herein, the influence of Gam1 on hsp40 and hsp70 expression was examined. The results of these studies establish that expression of Gam1 induces the elevation and relocalization of hsp70 and hsp40. Gam1-negative CELO is replication-defective; however, the present invention demonstrates that Gam1 function can be partially replaced by either heat shock or forced hsp40 expression. Thus, an essential function of Gam1 during virus replication may be to activate host heat shock responses with hsp40 as a primary target. By activating hsp40, which can act at least partially as a substitute for Gam1, the virus may be ensuring that replication is not limited by the amount of available Gam1.

The requirement of HSPs for virus replication does not appear to be restricted to the CELO adenovirus described herein. Data presented herein also demonstrate that hantavirus replication is associated with HSPs and with stress responses and further demonstrate that blocking hsp70 induction with an HSP inhibitor also blocks hantavirus replication. The data also suggest that HSPs, in some circumstances, may be rate-limited determinants for virion production.

The Materials and Methods used in the present study are now described.

CELO Adenovirus and General Methods

Modified Adenoviruses

To generate AdGam1, the Gam1 coding sequence (nucleotides 37,391-38,239 in the CELO Genome) (Chiocca et al., 1996, J. Virol. 70:2939-2949) plus an amino-terminal Myc tag were amplified from pSG9mGam1 (Chiocca et al., 1997, J. Virol. 71:3168-3177) using PCR and were transferred into an E1/E3 negative adenovirus 5 genome using homologous recombination in bacteria (Chartier et al., 1996, J. Virol. 70:4805-48 10; Michou et al., 1999, J. Virol. 73:1399-1410). The final virus bears an expression unit containing a cytomegalovirus (CMV) promoter, the Myc-tagged Gam1 coding sequence and a rabbit β-globin intron poly(A) signal embedded in the E1 region. Similar methods were used to construct Adhsp40 (containing a human hsp40 cDNA) (Ohtsuka, 1993, Biochem. Biophys. Res. Commun. antibodies (Dako Corp., Carpinteria, Calif.) and ECL reagents (Amersham, Arlington Heights, Ill.). An adenovirus bearing a human hsp 70 coding sequence (Hunt and Morimoto, 1985, Proc. Natl. Acad. Sci USA 82:6455-6459) with a tet-repressible CMV promoter will be described elsewhere. Control E1-negative AdS viruses expressing luciferase (AdLuc) and enhanced green fluorescent protein (eGFP) (AdEGFP), or β-galactosidase (AdRSVβgal) (Stratford-Perricaudet et al., 1992, J. Clin. Invest. 90:626-630), and the purification of adenovirus on CsCl gradients have been described (Hunt and Morimoto, 1985, Proc. Natl. Acad. Sci. USA 82:6455-6459).

CELOdg Construction

CELO with deleted Gam1 gene (CELOdG) was constructed using a deletion/recombination method (Michou et al., 1999, J. Virol. 73:1399-1410). In brief, a fragment of the CELO genome was manipulated to delete a SmaI/BglIII fragment (CELO nucleotides 36,818 to 37,972) and insert a luciferase expression cassette. This deletion removes the first 602 bp of the 875 bp Gam1 coding sequence. The modified fragment was assembled into a complete CELO genome to generate pCELOdG (paIM65). The luciferase expressing, wild-type Gam1 (CELOwt) and the CELO virus purification on CsCl gradients have been described (CELO AIM46; Michou et al., 1999, J. Virol. 73:1399-1410). The CELOdGhsp40 and CELOdGHsp70 genomes were constructed by exchanging the CMV/luciferase/β-globin cassette for CMV/hsp40/β-globin or CMV/hsp70/β-globin cassettes.

Analysis of the replication of CELOdG was performed by transfecting the CELOdG genome into LMH cells alone, or followed by infection with 1,000 particles per cell of AdGam1 after 24 hours. After 5 days, cells were collected and assayed for luciferase activity. An additional culture was used to infect a fresh set of LMH cultures either alone, or with AdGam1. Cell collection, luciferase assay and passages were repeated every 5 days for 5 passages.

Immunoblotting Analysis

A549 cells were lysed in lysis buffer (150 mM NaCl, 50 mM Tris, pH 7.5, 5 mM EDTA, 1% NP-40 containing protease inhibitor cocktail; Sigma). Cells were agitated at 4° C. for 30 minutes, passaged through a 25 gauge needle 5 times, sonicated in a bath sonicator for 5 minutes, and centrifuged at 14,000 r.p.m. (Eppendorf) for 5 minutes, and the supernatant was used for immunoblotting analysis. Equal quantities of protein (measured by Bradford reagent, Pierce Chem. Co., Rockford, Ill.) were resolved by PAGE, transferred to nitrocellulose and probed with the indicated antiserum preparations (see below). Antibody binding was revealed using the appropriate peroxidase-conjugated secondary antibodies (Dako Corp., Carpinteria, Calif.) and ECL reagents (Amersham, Arlington Heights, Ill.).

Immunofluorescence

Cells were plated on glass cover slips (12×12 mm) in 6-well dishes 24 hours before transfection or infection. Cells were fixed one day after transfection/infection in 4% parafornaldehyde for 15 minutes, rinsed 3 times with PBS, permeabilized with PBS/0.1% Triton X-100 (PBT) for 15 minutes, blocked in 5% BSA/PBT for 30-60 minutes, incubated with primary antibody for 15 minutes in 5% BSA/PBT, washed 3 times with PBT, incubated with secondary antibody for 15 minutes in 5% BSA/PBT, washed 2 times with PBT, washed 2 times with PBS, and mounted in 50% glycerol/PBS, 10 mM Tris pH 8.5, 4% n-propyl gallate (Sigma Chemical Corp., St. Louis, Mo.); all incubations were done at room temperature. DNA was stained with Hoechst dye. Images were acquired with a cooled CCD camera (Spot II: Diagnostic Instruments, mounted on an Axiovert microscope (Zeiss, Thornwood, N.Y.) equipped with 63×/1.4 lens with filters from Chroma Tech and processed using Adobe Photoshop software.

The following antiserum preparations were used: murine monoclonal 9E10 recognizing the Myc epitope; murine monoclonal RPN-1197 recognizing hsp70 (Amersham, Arlington Heights, Ill.) or goat polyclonal sera recognizing hsp70, hsp40, hsp90α, hsc70 and hsp27 (Santa Cruz Biotechnology, Santa Cruz, Calif.); anti-tubulin (Clone DMIA, Sigma Chemical Corp. St, Louis, Mo.); and a rabbit polyclonal directed against total capsid proteins. The following secondary antibodies were used at 1:100 dilutions: DTAP conjugated donkey anti mouse, DTAP conjugated donkey anti rabbit and Cy3 conjugated donkey anti goat (Jackson Laboratories, Bar Harbor, Me.).

Transfection of cells was conducted using a double PEI technique as described (Michou et al., 1999, J. Virol. 73:1399-1410). LMH cells (Kawaguctu et al., 1987, Cancer Res. 47:4460-4464) and A549 cells (ATCC CCL-185) were cultured in DMEM plus 10% FCS (DMEM, 2 mM glntamine, 100 IU penicillin, 100 μg/ml streptomycin and 100% (v/v) fetal 30 calf serum). The 293 cell line (Graham et al., 1977, J. Gen. Virol. 36:59-74) was cultured in MEMalpha with 10% newborn calf serum.

Other methods which were used but not described herein are well known and within the competence of one of ordinary skill in the art of virology and of cellular and molecular biology.

The Results of the experiments described in this example are now presented.

An E1-defective adenovirus 5 vector (AdGam1) was constructed to direct substantial levels of Gam1 expression (FIGS. 1a and 1b). To determine whether Gam1 expression influences hsp70 protein levels, A549 cells were infected with either AdGam1 or control adenoviruses encoding nuclear-targeted β-galactosidase (Adβgal), luciferase (AdLuc) or enhanced green fluorescent protein (AdEGFP). The virus-encoded transgene products were expressed to various levels and were detected by Coomassie staining (FIGS. 1a and 1b, left panels). A substantial increase in hsp70 protein occurred in cells that expressed Gam1 (FIGS. 1a and 1b, right panel) or that were exposed to heat shock (FIG. 1a, right panel), but not in cells that were infected with the same amount of control adenoviruses Adβgal, AdLuc, or AdEGFP in the absence of heat shock (FIGS. 1a and 1b). Gam1-directed hsp70 induction was similar to that obtained with heat shock (FIG. 1b, right panel). AdGam1 infection, but not AdLuc infection, also led to increases of hsp40 and hsp27 but not to significant increases in hsc70 or hsp90α (FIG. 1c).

These results demonstrate that vector infection itself, background gene expression from the vector, and exogenous protein expression per se do not alter hsp70 levels.

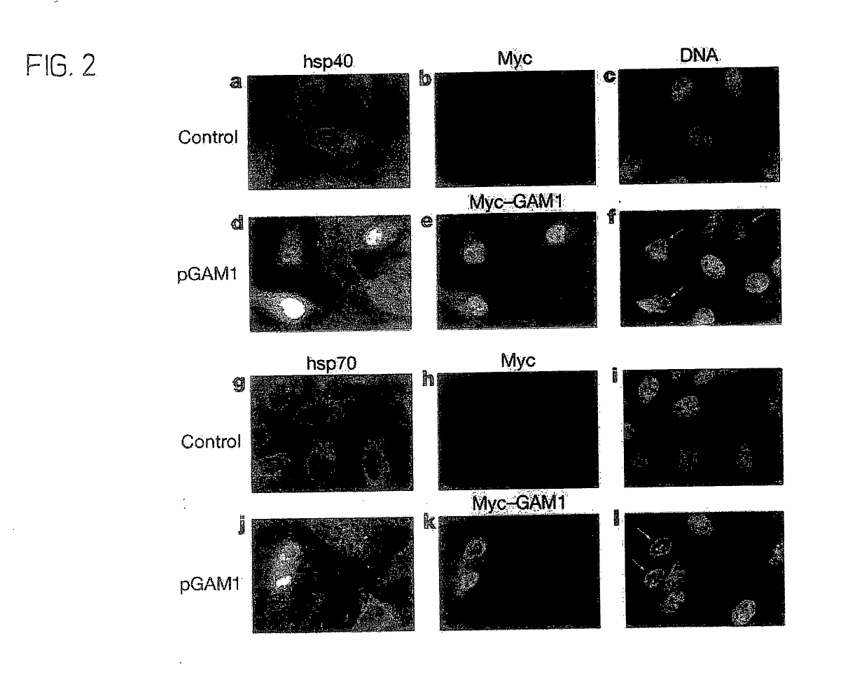

Hsp40 and hsp70 are known to translocate to the nucleus following heat shock (Welch and Feramisco, 1984, J. Biol. Chem. 259:4501-45 13; Hattori et al., 1992, Cell. Struct. Funct. 17:77-86). The effect of the adenovirus Gam1 protein on this function was not known. It was next determined whether Gam1 transfected into cells could influence both hsp40 expression and hsp70 expression, and whether Gam1 influenced intracellular movement of these proteins. Cells (A549) were either non-transfected (controls) or were transfected with pGam1. Then, hsp40 and hsp70 expression were determined bit immunofluorescence analysis. As demonstrated in the photomicrographs, non-transfected (control) A549 cells presented predominantly cytoplasmic hsp40 and hsp70 (FIGS. 2a-2c and 2g-2i). However, it was found that expression of Gam1 protein in transfected A549 cells caused an elevation and nuclear relocalization of both hsp40 (FIG. 2d-2f) and hsp70 (FIGS. 2j-2l).

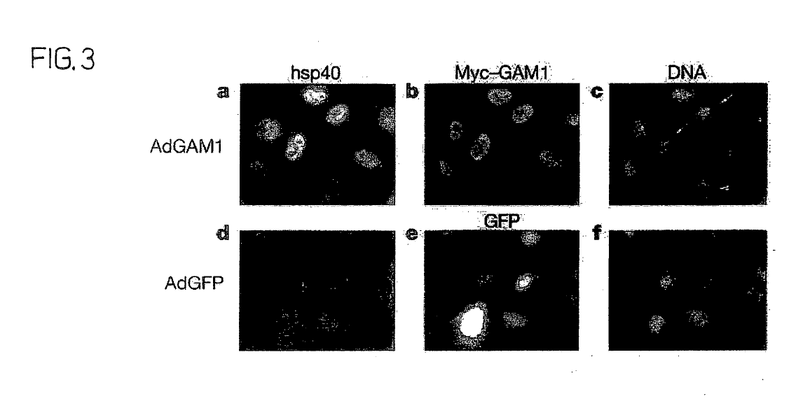

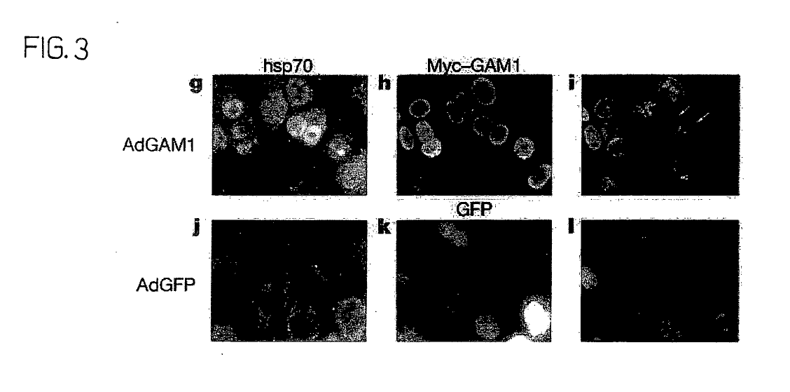

It was next determined whether expression of Gam1 protein following actual infection with a Gam1-containing virus would have an effect on hsp40 and hsp70 expression and localization similar to the effects demonstrated in Example 2. Cells (A549) were infected with AdGam1 or a control, AdEGFP. Infection with AdGam1, as with transfection with pGam1 in FIG. 2, led to elevation and nuclear relocation of hsp40 (FIGS. 3a-3c) and hsp70 (FIGS. 3g-3i). Neither transfection with a control plasmid (pEGFP) nor infection of cells with a control AdEGFP expressing comparable levels of protein (FIG. 1b) altered hsp40 or hsp70 levels or localization (FIGS. 3d-3f and 3j-3l).

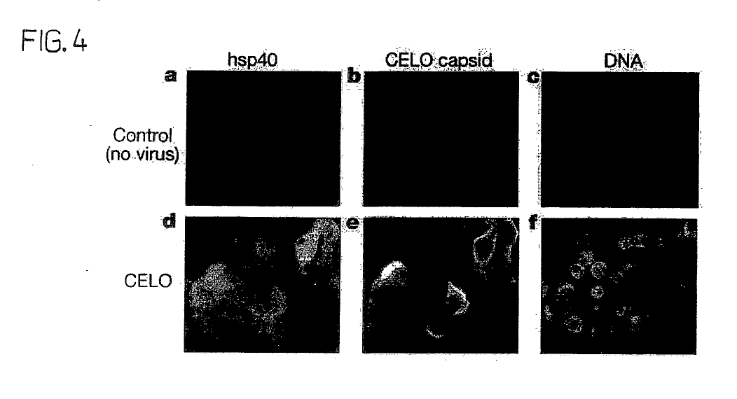

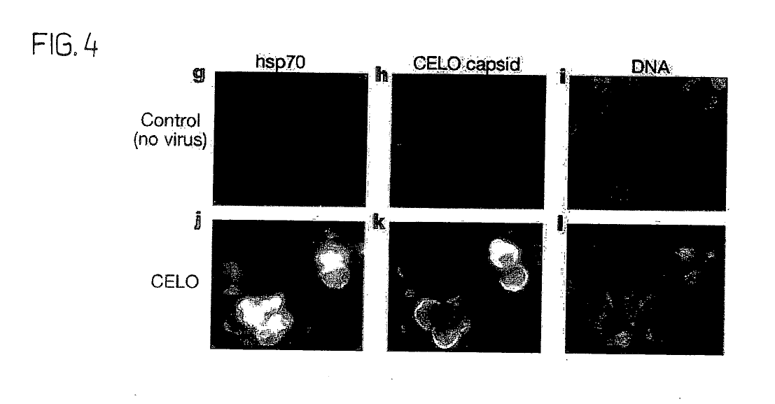

The effects of the parent CELO adenovirus infection and Gam1 expression on hsp40 and hsp70 expression were next tested on LMH cells. Control (non-infected) and infected cells were subjected to immunofluorescence analysis. The photomicrographs reveal that hsp40 and hsp70 levels increased during CELO replication in LMH cells compared to non-infected cells (FIG. 4), as they did in the A549 cells described in FIG. 3.

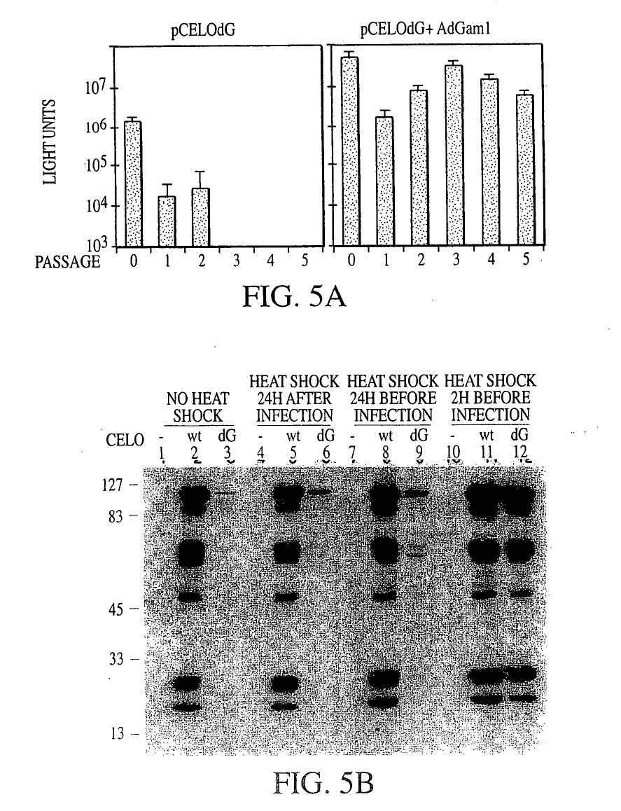

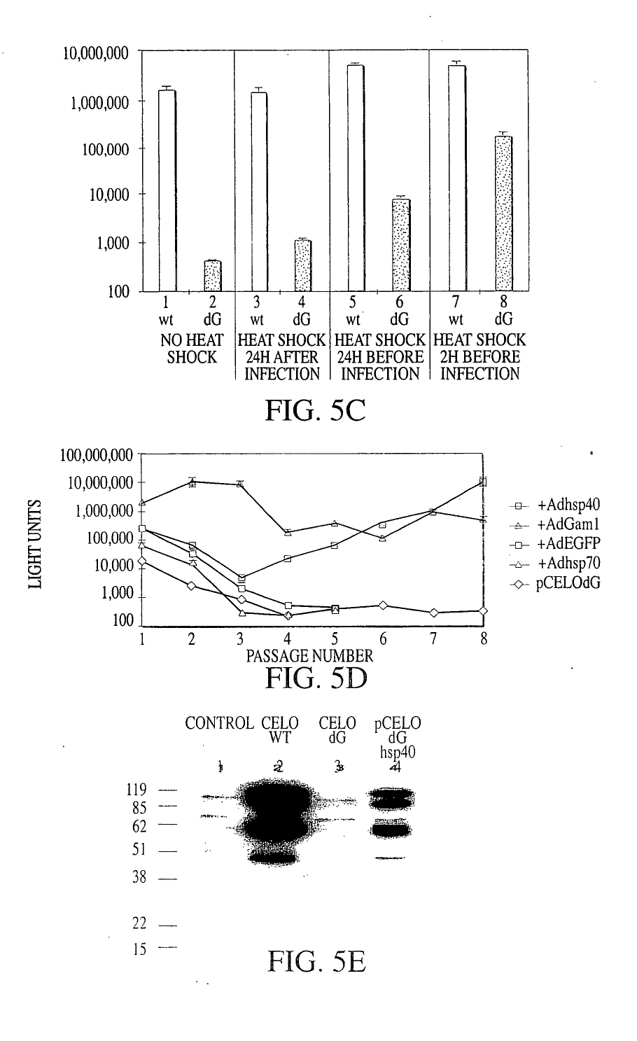

To examine the requirement for Gam1 in CELO replication, a Gam1-negative CELO genome bearing a luciferase gene (CELOdG; see Methods) was prepared. The CELOdG genome yielded replicating virus when complemented with AdGam1 (FIG. 5a), but not with a control AdEGFP (FIG. 5c). Thus, the Gam1 gene is required for CELO growth in LMH cells. In chicken embryos, wild-type CELO (CELOwt) produces a maximum virus yield with inocula of 4×10¹ particles and higher; full replication of CELOdG is only observed with inocula of 4×10⁷ particles and greater, demonstrating the requirement for Gam1 during in vivo virus replication.

To determine whether activating a heat shock response is a necessary function of Gam1, heat shock was tested for its ability to replace Gam1 in CELO replication. When the genome of CELOdG was transfected into LMH cells and the culture exposed to 45° C. for 90 minutes, viral growth was observed. Heat shock complementation was used to amplify CELOdG, and then purified CELOdG virus was compared with CELOwt in a single cycle of virus replication by measuring the production of capsid proteins (FIG. 5b) or the production of virus particles capable of delivering a luciferase gene (FIG. 5c). The production of CELOdG, which is clearly defective in non-heated cultures (FIG. 5b, lane 3; FIG. 5c, lane 2), was stimulated by heat shock (FIG. 5b, lanes 6, 9 and 12; and FIG. 5c, lanes 4, 6 and 8), with the greatest stimulation observed when the heat shock was applied 2 hours before virus infection (FIG. 5b, lane 12; FIG. 5c, lane 8).

The heat shock used for complementation might influence a variety of cellular proteins in addition to hsp40 and hsp70. To determine whether either or both hsp40 and hsp70 are essential Gam1 targets for CELO replication, recombinant adenoviruses were prepared directing the expression of these two heat shock proteins (Adhsp40, Adhsp70) and tested them for complementing CELOdG growth in LMH cells. Overexpression of hsp40, but not hsp70, was found to support CELOdG growth (FIG. 5e). CELOdG genomes were also constructed that contained either hsp40 or hsp70 expression cassettes in place of the Gam1 gene (CELOdGhsp40, CELOdGhsp70). Introduction of the CELOdGhsp40 genome into LMH cells yielded replicating virus: the CELOdGhsp70 genome, like the parental CELOdG, was not viable (two independent genomes were tested. In a single cycle of replication, CELOdGhsp40 was found to replicate at roughly one-twentieth of the level of CELOwt, whereas CELOdG growth was not detectable (FIG. 5f). Thus, hsp40 overexpression alone is sufficient to at least partially replace Gam1 function.

The present invention demonstrates that the CELO Gam1 protein increases the cellular levels of hsp40 and hsp70 and relocates these proteins to the nucleus. To date, the CELO virus is unique in having evolved a replication strategy using a single gene product, Gam1, which induces a heat shock response in the host cells, essential for the replication of the virus. Gam1 is required for virus replication, and, moreover, the requirement for Gam1 in virus replication can be partially met either by heat shock or by overexpression of hsp40. The data support the idea that the induction of a heat shock response is an essential viral function for virus replication and is not merely a cellular adaptive response to infection. The relocalization of hsp40 and hsp70 to the nucleus by Gam1 probably facilitates the protein reorganization required early in virus replication. Heat shock induction is also observed during the replication of several viruses including herpes simplex, cytomegalovirus and adenovirus, suggesting that heat shock protein function in virus replication may serve as a common mechanism for all of these viruses.

### Example 2

### Hantavirus

Hantavirus Methods

Hantavirus

Sin Nombre (SN) virus is the primary etiologic agent of hantavirus cardiopulmonary syndrome (HCPS) in the New World (Mertz et al., 1998, Dis. Mon. 44:85-138; Schmaljohn and Hjelle, 1997, Emerg. Infect. Dis., 3:95-104). This laboratory utilizes the SN virus isolate SN77734 (Botten et al., 2000, Proc. Natl. Acad. Sci. USA 97:10578-10583). This isolate was obtained through passage of tissue homogenates from a seropositive mouse collected at the same site as were the colony founders. Although SN virus is notoriously difficult to isolate, by passage 2 stocks with titers of >1×10⁴ had been prepared that would infect 100% of deer mice inoculated by the intramuscular route.

Deer Mice

The deer mouse (Peromyscus maniculatus) is the reservoir host for the Sin Nombre virus (hantavirus). The mice were maintained in a containment facility comprising an outdoor colony of artificial burrows as described (Botten et al., 2000, Proc. Natl. Acad. Sci. USA 97:10578-10583; Botten et al., 2000, J. Mammal. 81:250-259). Mice were maintained in the outdoor laboratory at ambient temperature.

Infecting Mice

Mice were generally inoculated intramuscularly in groups of 5-7 with 5 deer mouse ID₅₀ of SN hantavirus isolate SN77734. Mice were inoculated in a separate quarantined laboratory that is isolated from control mice. SN virus distribution has been shown to include virtually all tissues as assessed by quantitative TaqMan RT-PCR and immunohistochemical detection of the viral N antigen. However the SN virus distributes most quickly and at highest titer to lung, heart, and brown adipose tissue (Botten et al., 2000, Proc. Natl. Acad. Sci USA 97:10578-10583).

Immunohistochemistry

The general techniques are described above. Hantavirus (SN) N antigen was detected by immunohistochemical staining using 1:10,000 to 1:5000 rabbit anti-SN N after antigen retrieval, followed by biotinylated anti-rabbit IgG, then SA-HRP with AEC developer. The antibody was prepared in this laboratory. A semiquantitative scoring system was used to establish the amount of viral antigen expression in infected animals. A scoring matrix was utilized with the scores 1+, 2+, 3+, and 4+. These scores were based, respectively, on signals of 1-5 cells/HPF, 6-5, 16-40, and >40.

Western Blot Analyses

Hantavirus studies were generally performed as described above. For tissue culture studies, after the specified intervals, the cells were trypsinized and counted and subjected to lysis with a Beadbeater in standard SDS-βmercaptoethanol lysis buffer. Typically, proteins from the equivalent of 2×10⁴ cells were loaded per lane and subjected to 12.5% SDS-PAGE and transferred to nitrocellulose. Membranes were probed with antibodies as described. For virion release experiments, measured by SN virus N antigen, typically 12 μl of supernatant was subjected to SDS-PAGE, then transferred to nitrocellulose and probed for N antigen using rabbit anti-SN N antigen (1:5000). A control lane loaded with 40 ng of recombinant N antigen was used as a control.

Antibodies

Rabbit anti-SN N antigen antibody was prepared in this laboratory. Heat shock protein antibodies were obtained from Stressgen, Victoria, B.C., Canada. Antibodies recognizing the hsp70 group include: the SPA812 rabbit polyclonal and the SPA810 mouse monoclonal, both of which recognize hsp72, an inducible form of hsp70; and SPA815 (rat monoclonal) and SPA822 (hsp72+hsc70) which recognize constitutively expressed forms of hsp70, namely hsc70 or hsp73. Other Stressgen antibodies against heat shock proteins used in the hantavirus studies include SPA400 (hsp40/DNA J), SPA804 (hsp60), SPA800 (small hsps-hsp27/28), and SPA830 (hsp90 (HtpG)).

Inducing Heat Shock/Stress

Heat shock was induced by subjecting confluent monolayers of control or persistently infected (SN virus) Vero E6 cells to 43° C. for 1.5 hours (Oglesbee et al., 1993, Virology 1 92:556-567). The cells were then harvested at various time points.

Inducing Cold Stress

Mice were maintained in an outdoor quarantine laboratory at ambient temperature. Temperatures were monitored to help establish daily fluctuations in temperature and records were kept of daily highs, lows, and average daily temperatures. Animals were sacrificed after cold snaps and the SN virus N antigen was measured to determine changes that occur due to cold stress.

Phenylephrine-Induced Stress

In the rat and mouse models it is possible to model the signals that activate brown adipose tissue during cold acclimation by administration of adrenergic agents such as phenylephrine. Phenylephrine was injected intraperitoneally at 25 μg/kg and animals were sacrificed by euthanasia at various time points.

Confinement Induced Stress

As an alternative to phenylephrine, some mice were subjected instead to six (6) hours of confinement in a metabolic cage, to induce a stress that might be less harmful to the mice than the potential hypothermia problems that might arise when stressed by phenylephrine.

Cell Culture

The Vero E6 African Green Monkey (AGM) cell line was used to study hantavirus in vitro and was maintained by standard techniques.

TaqMan RT-PCR Assay

The technique used was based on that previously described (Bharadwaj et al., 2000, J. Infect. Dis. 182:43-48; Botten et al., 2000, Proc. Natl. Acad. Sci. USA 97:10578-10583). Briefly, primers closely spaced together on the SN virus S segment were used and an internal 6-fam (6-carboxyfluorescein)-labeled (FAM-labeled) oligonucleotide probe with carboxytetramethylrhodamnine (TAMRA) quencher on the 3′ end. Threshold cycle number, C_{T}, was measured, which is inversely related to the log-linear copy number, as assessed by a standard curare of template. The dynamic range of this assay is from 5 to ≧5×10⁷ copies. The reactions were conducted in two tubes, one for RT using random hexamer primers, and then a separate tube for PCR using specific primers. The assays ere performed in triplicate and the results averaged. For a run to be valid, the R-value between −log (copies of std curve target) and C_{T} must be −0.995 or less. Hexamers inhibit PCR, so an aliquot (5%) of the RT mix for the PCR tube wvas used, to reduce the sensitivity after dilution to ˜500 copies per mg of tissue.

Viral Focus Quantitation

Four-fold dilutions of clarified supernatant were added in duplicate to confluent monolayers of Vero E6 cells in 48 well plates. After one hour the cells were overlaid with complete media that had been made viscous with 1.2% methylcellulose. After 7 days the cells were fixed in 100% methanol containing 0.1% H₂O₂, then stained with rabbit anti-N (1:5000), followed by HRP-conjugated goat anti-rabbit Ig and then DAB substrate (Bharadwaj et al., 2000, J. Infect. Dis. 182:43-48; Botten et al., 2000, Proc. Natl. Acad. Sci. USA 97:10578-10583). The number of foci in a well is multiplied by the dilution factor of the supernatant in that well to determine the titer of focus forming units/ml.

Hantavirus (Sin Nombre) Examples

Another goal was to determine whether hsp70, or other heat shock proteins or pathways, directly regulates the replication of hantaviruses in vivo and in vitro. One of the goals in developing the deer mouse infection model was to study the mechanisms by which SN virus establishes persistent infection, and how the virus might reactivate after establishing persistence.

The genome of hantaviruses consists of three segments of negative polarity: a large L segment that encodes the RNA-dependent RNA polymerase, an M segment that encodes the envelope glycoproteins G1 and G2, and an S (small) segment that encodes the viral nucleocapsid (N) antigen. To determine whether SN virus (hantavirus) persisted in the tissues of infected deer mice, juvenile deer mice were inoculated with 5 deer mouse ID₅₀ of SN77734, and necropsies conducted at 60, 90, 120 and 180 days. Immunohistochemical analyses were used for the viral N antigen in conjunction with a semiquantitative scoring system to establish the degree to which viral N antigen was downregulated or extinguished in the course of persistent infection. In the 14 tissues examined, the mean antigen staining scores did not decline substantially between 35 and 60 days, but between 60 and 90 clays, there was a marked diminution in antigen score. Viral antigen was routinely detected only in heart after 90 days, although, as described previously, viral RNA was present in brown fat as well. This pattern persisted at the 120 day time point, but four days before the 180 day time point, there was a cold snap at the outdoor quarantine lab, during which the nighttime temperatures declined by nearly 10° C. At this time point, there was a marked increase of SN virus antigen scores, which were tentatively attributed to the precipitous change in ambient temperature.

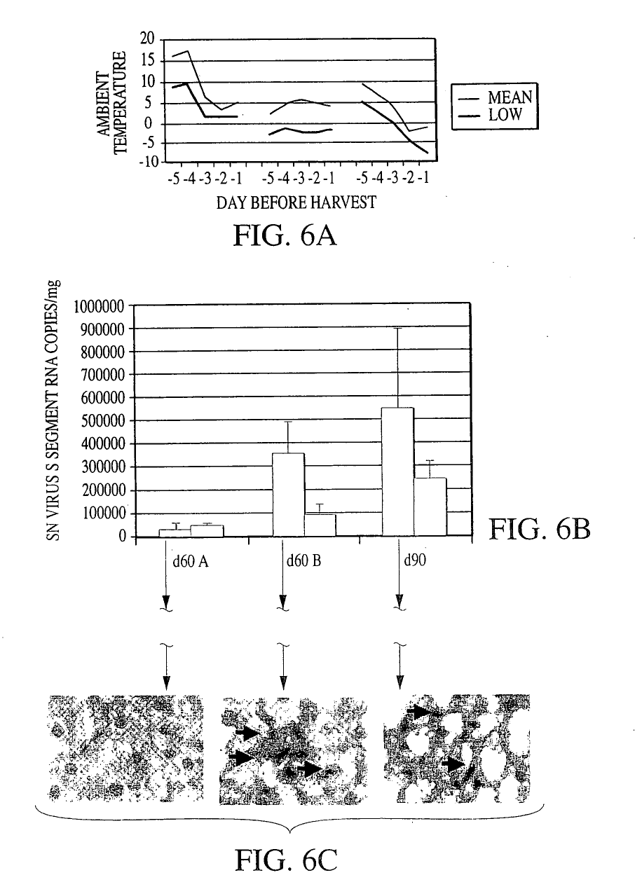

By comparison to deer mice that are inoculated as juveniles, those that are inoculated as adults reach the persistency stage more rapidly. By 60 days, adult deer mice show very low antigen expression and markedly reduced levels of SN virus RNA in tissues. At the time, tissues had been collected from a set of adults at 60 days post-infection during the fall (October), at a time when the mean temperature was 10° C. To test the effects of temperature on viral antigen expression, groups of adult deer mice were inoculated during the winter when the temperatures had markedly declined, for harvesting at 60 and 90 days post-infection. For this study, TaqMan RT-PCR assays were conducted on BAT and heart tissues, as was IHC staining for N antigen (FIG. 6). The temperature readings from a weather station 100 meters from the quarantine facility for the 5 days preceding the harvest of the tissues showed that there was a significant difference in ambient temperature between the first group of animals examined at 60 days (group d60A) compared with the other two groups. Whereas the median antigen score in BAT was 1+ in the warmer group d60A, it had increased to 3+ in the two colder groups d60B and d90.

FIGS. 6A and 6B are aligned vertically to allow the temperature plot in A to be linked to specific experimental groups in B. FIG. 6A shows the ambient temperature at the laboratory in the 5 days immediately preceding the point at which the animals were euthanized. The ambient temperatures experienced by group 60A remained warm through the experimental period, never going below 0° C. The antigen load in this group remained low (a representative (negative) field from IHC is shown in C (top micrograph, 400× in original). However, the ambient temperatures experienced in groups d60B and d90 were substantially lover, with overnight lows below freezing. In these latter groups, viral RNA load (FIG. 6B) was very high, at least equivalent to that seen in acute infection. The induction of SN virus replication was most prominent in BAT but reactivation was also seen in heart, either as a primary event or possibly as a result of secondary spread of virus from BAT or other tissues.

HSPs are activated in deer mice by signals that favor activation of brown adipose tissue (BAT). In the rat and mouse models, it is possible to model the signals that activate BAT during cold-acclimation by administration of adrenergic agents such as phenylephrine. To test whether this observation is true also in the deer mouse model, adult deer mice in the University of New Mexico (UNM) colony were exposed to 25 μg/kg phenylephrine via the intraperitoneal route. As an alternative stressor, perhaps less likely to cause fatal hypothermia in deer mice housed at the quarantine lab, deer mice were also subjected to the stress of being in a confined space (a metabolic cage) for 6 hours. Six (6) hours after the stress, necropsies were conducted and tissues stained for hsp25, 47, 60, and 70, as well as with an antibody (Stressgen) that reacted with both hsc70 and hsp70. Of the antigens studied, only hsp70 was induced in the BAT and liver of stressed deer mice, as assessed by IHC staining and/or western blotting. The antibody that also detects hsc70 showed widespread staining in all tissues and induction could not be discerned. Hsp25 showed an induction of approximately 2-fold in lung, and hsp70 showed a 2-3 fold induction in the large airways in stressed deer mice.

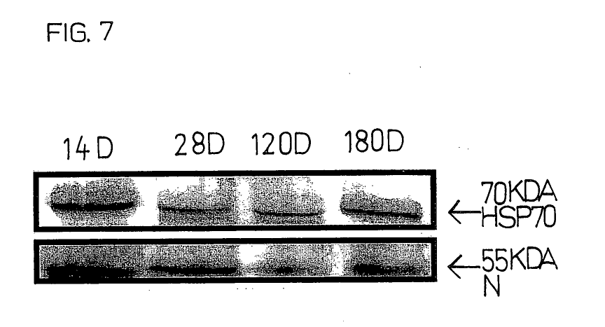

Next it was determined whether an HSP response occurred in BAT of infected mice in response to cold stress. In FIG. 7 it can be seen that hsp70 levels and viral N antigen levels increased in BAT of infected deer mice following cold stress. Viral N antigen levels were high in acute infection but had declined by 120 days. However, temperatures declined markedly 4 days before the 180 day timepoint, and SN viral antigen levels increased as measured by IHC.

While it appears that hsp70 and N antigen levels moved in lockstep in these studies of single animals, it is possible that HSP levels peaked before virus replication was stimulated and N antigen began to increase.

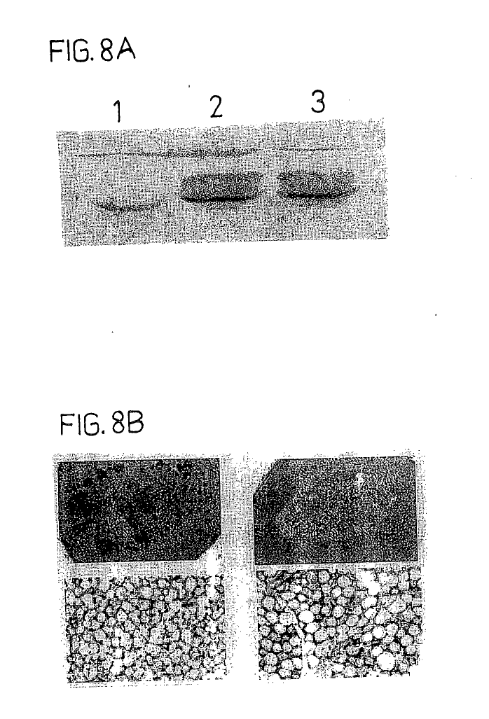

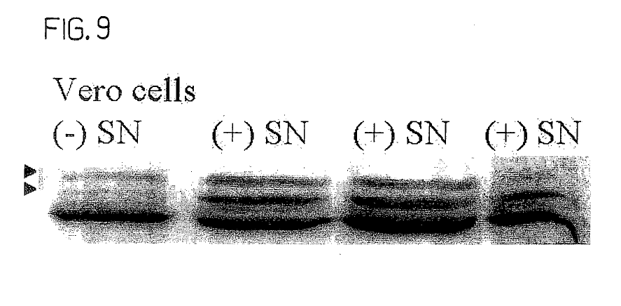

It was then determined whether HSPs could be experimentally induced in other tissues of mice. Mice ere treated with the stressor phenylephrine (25 μg/kg) or were subjected to stress by placement in a metabolic cage. Western blot analyses showed that after 6 hours of phenylephrine-induced stress or metabolic cage-induced stress hsp72 expression had increased in the liver compared to untreated mice (FIG. 5A). Immunohistochemical analyses of hsp70 expression demonstrated increased levels of expression in adrenal glands and BAT after 6 hours of phenylephrine-induced stress (FIG. 8B). DAB stain was noticeably darker in adrenal glands and BAT (FIG. 8B, right panels) of stressed animals compared to those tissues in control animals (FIG. 8B, left panels). These and other data presented herein suggest that several kinds of stress may induce a heat shock type of response, i.e., an increase in HSPs.

Experiments were performed to determine the effects of hantavirus infection (strain SN) on HSP expression in cultured cells. To address the hypothesis that infection with SN virus induces hsp70 or hsc70 expression in vitro, the expression of hsp70 in uninfected African Green Monkey (AGM) Vero E6 cells was compared with that in Vero E6 cells that were persistently infected with either SN77734 or the California isolate of SN virus, CC107. Persistent infection with both isolates was associated with a several-fold induction of what was tentatively identified as the constitutive form of hsp70, hsc70, which, in Vero E6 cells as elsewhere, migrates at slightly higher molecular mass than does the inducible form, hsp70 (FIG. 6). Western blot analyses, using an hsp70 specific antibody which does not bind to hsc70 under the conditions used, demonstrated that there was no reaction with the middle band, suggesting that the induced form of hsp70 in infected Vero E6 cells is hsc70. However, in a separate study it was found that hsp72 itself is induced in infected or uninfected Vero E6 cells from the same very low basal level by heat shock. The observation that heat induces hsp72 but the virus induces hsc70 would not be totally unexpected, because hsc70 is induced or associated with virus in preference to hsp70 in several types of viral infections (Sainis et al., 1994, FEBS Lett. 355:282-286; Saphire et al., 2000, J. Biol. Chem. 275:4298-4304). Thus, hsc70 is constitutively overexpressed in persistently infected Vero E6 cells.

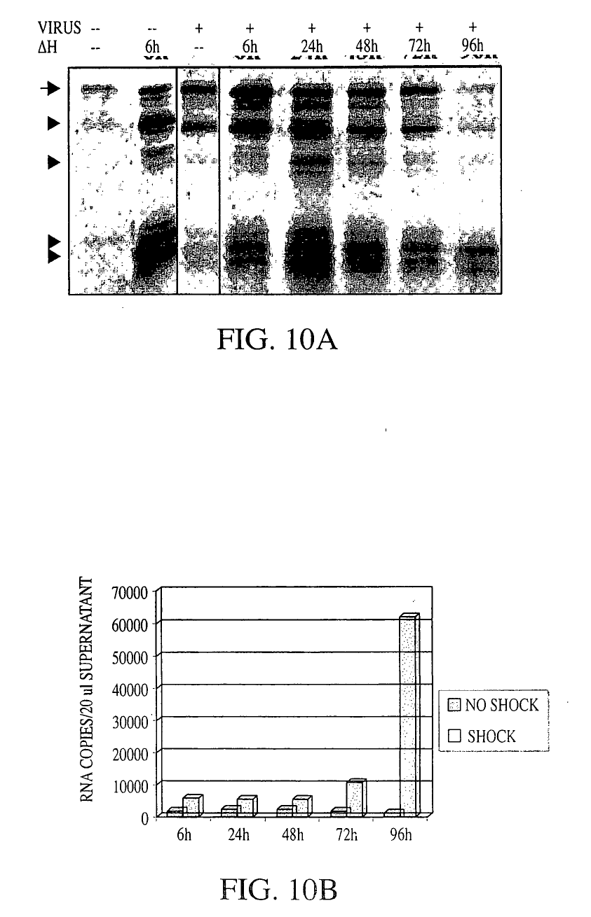

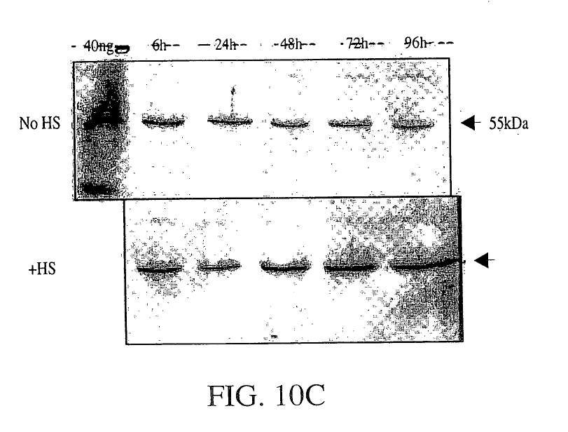

A series of experiments were performed to determine whether persistent hantavirus infection of Vero E6 cells influenced basal and/or inducible levels of HSPs and whether infected cells responded to heat stress by activation of virus. FIG. 10A shows that hsp70 can be induced after heat shock of Vero E6 cells, with or without the presence of SN virus. Thus, HSP is induced by thermal stress, even in persistently infected cells. Unfortunately, there appears to have been proteolysis during this particular study despite the presence of aprotinin and iodoacetamide, so the hsp70 antibody reactivity of the breakdown products (short arrows) should be “added” to that of the 70 kDa band (long arrow) in interpreting the overall hsp70 signal. These results show that SN virus does not inhibit the HSP response to thermal shock, but that its presence sets the background level of HSP higher.

To address the question of whether induction of hsp70 causes reactivation of SN virus from cells, persistently-infected (SN77734) cells were subjected to heat shock at 43° for 1.5 hours, and then supernatant was sampled for viral RNA by TaqMan quantitative RT-PCR assay. There was an approximately 2-3 fold increase in viral RNA at 6, 24, and 48 h, but by 72 hours the increase was approximately 6-10-fold, and by 96 hours there was an approximately 30-fold increase (FIG. 10B). This experiment shows that persistent infection and reactivation of SN virus in cultured cells can be modeled, and that SN virus is reactivated by heat stress. To the extent that this reactivation can be ascribed to an hsp70-class chaperone, these results implicate hsp72, the heat shock-inducible form, rather than hsc70, which is not considered to be heat-responsive. Western blot analyses also showed virion (SN virus N antigen) release into the supernatant following heat shock treatment (FIG. 10C, lower panel) compared to untreated cells (FIG. 10C, upper panel). Increased levels of N antigen are apparent by 72-96 hours. No increase in released hsp70 was detected.

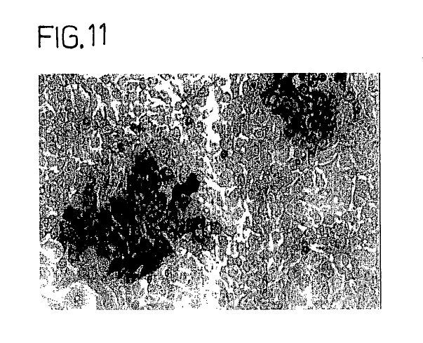

Inhibitors of heat shock protein pathways could potentially inhibit both infection of uninfected cells and reactivation of virus in infected cells. Assays to measure infectious SN virus would be useful for determining amounts of infectious virus present and changes resulting from manipulating HSPs or HSP regulatory pathways. These assays include western blot analyses, ELISA with anti-nucleocapsid antibody, quantitative TaqMan RT-PCR for viral RNA, and focus assays for virus production in the supernatant. TaqMan assays are currently in use (Botten et al., 2000, Proc. Natl. Acad. Sci. USA 97:10578-10573), and here it is shown that infectious SN virus can be quantitated by focus assay using antibody to N antigen. SN viral foci can be seen as dark brown clusters in the micrograph in FIG. 11. Thus, both TaqMan and focus assays can be used to measure infectious SN hantavirus and the effects of various inhibitors on the virus (Botten et al., 2000, Proc. Natl. Acad. Sci. USA 97:10578-10573).

Because the new data described herein suggested a relationship between heat shock, HSPs, viral infection, and reactivation, the question of whether a known inhibitor of HSP induction would effect the induction of hantavirus RNA expression was addressed. The ability to inhibit virus infection or virus reactivation by inhibiting HSPs would be of great benefit. It can be seen in FIG. 12 that the flavonoid quercetin, an inhibitor of HSP induction, inhibits the heat shock induced reactivation of SN hantavirus. Infected Vero E6 cells were subjected to heat shock (43°, 1.5 hours) with or without quercetin at 100 μM. SN viral RNA released into the medium was measured every 2 days for 10 days. It can be seen that heat shock induced high levels of viral RNA titers by day 10, but cells that were heat shocked and treated with quercetin did not have increased levels of viral RNA titers. In fact, the titers in the heat shocked cells treated with quercetin were similar to those cells that were not subjected to heat shock. There was a statistically significant 7.3 fold inhibition (p=0.0003) of viral RNA titers in quercetin treated cells, compared to the statistically significant (p=0.013) 4.7 fold difference between cells subjected to heat and those not subjected to heat shock. Temporal changes in induction of viral RNA compared to other experiments may be due to slight differences in experimental conditions such as differences between freshly infected cells and persistently infected cells.

### Example 3

### Human Immunodeficiency Virus 1 (HIV-1)

HIV-1 Methods

Cell Lines and Antibodies

HeLa CCL-2 cells (American Type Tissue Culture Collection) were used for analysis of HIV-1 particle release. Cells were grown in monolayer cultures at 37° C. in Dulbecco's modified Eagle's medium (DMEM; GIBCO, Gaithersburg, Md.) supplemented with 5% fetal bovine serum (FBS; Hyclone Laboratories, Logan, Utah) and maintained using standard techniques. Polyclonal antibodies to HIV Gag and UBP were described previously (Callahan et al., 1998, J. Virol. 72:5189-5197, published erratum appears in 1998, J. Virol. 72:8461). The anti-hsp70 and anti-hsp90 monoclonal antibodies were purchased from Stressgen (Victoria, Canada). The anti-GST polyclonal antibody was obtained from Santa Cruz Biotechnology (Santa Cruz, Calif). Goat anti-rabbit and anti-mouse antibodies conjugated with alkaline phosphatase were purchased from Sigma Immunochemicals (St Louis, Mo.).

DNA Constructions and Proteins

Several of the plasmids used including pGST-UBP have been described previously (Callahan et al., 1998, J. Virol. 72:5189-5197, published erratum appears in 1998, J. Virol. 72:8461). Derivatives of pGST-UBP contained the following UBP segments: Δ1-93, Δ95-195, Δ288-313, N1/2 (a.a. 1-145), C1/2 (a.a. 145-313), and TPR2-4 (a.a. 288-313). For yeast UBP knockout studies, the entire y-UBP gene (Genbank Accession: U43491) was directly cloned into cloned into the Bam HI site of pRS316. Yeast UBP including the flanking non-translated regions was amplified with the following primers: upstream, 5′-CGCGGATCCAGAAGATTCCAGGTTCAAG-3′, SEQ ID NO: 4, downstream, 5′-GCTGGATCCAGTTCTATACAGATTTACAT-3′, SEQ ID NO: 5, where the Barn HI sites are underlined. The resulting construct was referred to as pRS316-y-UBP. To generate the y-UBP gene targeting construct, pRS316-y-UBP was digested with Bgl II at nt 751 of the UBP ORF and a histidine biosynthetic marker gene flanked by Bam HI sites was directly ligated to the Bgl II ends. This resulted in a targeting construct (pRS316-His-y-UBP) in which the His marker was flanked by more than 1 Kb of UBP DNA on each end. To produce purified UBP, a vector expressing histidine tagged-UBP (pHis-UBP) was created by cloning the UBP ORF directly into the pQE30 vector via the Bam HI and Hind III restriction sites. For dominant interference experiments, either wt UBP, N1/2 (a.a. 1-145), or a fragment containing TPR 2-4 with flanking charged residues (FTPR; a.a. 81-209) were co-expressed. All of these plasmids were similarly constructed, for example, the FTPR plasmid was created by amplification of the region from nt 241-627 with the following primers: upstream, 5′-GACTGCGCGCAGAAGGAGAGAGATGACCCCGCCTTCCGAG-3′, SEQ ID NO:6, downstream, 5′-GCAGGCTAGCTTAGGCCTCCCGCAGCTTC-3′, SEQ ID NO:7, where underlined nucleotides represent Bss HII and Nhe I sites respectively. All of the derivatives were cloned into pBG139 such that they were under the control of the LTR as described previously (Callahan et al., 1998, J. Virol. 72:5189-5197, published erratum appears in 1998, J. Virol. 72:8461). For particle release experiments a full-length HIV Vpu⁺ construct (MSMBA) or Vpu⁻ construct (pDF101) were used.

Protein Purification

GST-UBP, used for far-western blots, was expressed in E. coli by standard techniques. Bacteria pelleted from a one liter culture were treated with 10 ml of lysis buffer (100 mM NaCl, 10 mM Tris [pH 8.0], 0.1 mM EDTA, and 0.5% Triton X-100). Lysozyme was added to a final concentration of 1.8 mg/ml and the mixture incubated for 30 minutes at 37° C. The lysate was then subjected to three freeze-thaw cycles. The protein was centrifuged at 10,000×g in a Beckman J2-21 centrifuge and the supernatant was recovered. The protein fraction was then applied to a glutathione-Sepharose column (Pharmacia, Upsala Sweden), washed, and eluted according to manufacturer's specifications. For ATPase and luciferase refolding assays, purified His-UBP was used. Similarly, His-UBP expressed in bacteria was applied to a 1 ml nickel-NTA resin column in the appropriate buffer as prescribed by the manufacturer (Qiagen, Chatsworth, Calif.). The column was washed and eluted with 400 mM imidazole. All purified proteins were analyzed by Coomassie gel and quantified by the Bradford assay (Bio-Rad, Hercules, Calif). Pure hsp70, hsc70 and hsp90 used in several experiments were purchased from Stressgen.

Western and Far-Western Blotting

Western blots were performed using standard techniques. Briefly, 30 μg HeLa cytoplasmic extract or 20 ng of hsc70, hsp70, or hsp90 were denatured in 1×SDS sample buffer. The samples were loaded on a 10% polyacrylamide gel. Separated protein was transferred to nitrocellulose membranes. The membranes were blocked in 5% dry milk protein in TBS (20 mM Tris [pH 7.4], 100 mM NaCl) for 45 min. The gels with proteins separated on them were transferred to nitrocellulose and blocked against 5% dry milk protein in TBS for 45 min. Primary antibody, either anti-hsp70 or hsp90 monoclonal antibodies, or anti-UBP rabbit polyclonal antibody was incubated with the blots at appropriate concentrations (1:1000 dilutions for each) in 5% milk protein in TBS for 2 hours at 4° C. with gentle rocking. The membranes were washed with TBS for 10 minutes. Secondary antibody, either alkaline phosphatase conjugated goat anti-mouse IgG or alkaline phosphatase conjugated goat anti-rabbit IgG diluted at 1:5000 in 5% Milk in TBS, was incubated with the blots for 2 hours at 4° C. with gentle rocking. The blots were washed in TBS then rinsed in AP buffer (100 mM Tris [pH 9.5], 100 nm NaCl, 5 mM MgCl₂). The blots were developed by incubation with NBT (nitroblue tetrazolium) and BCIP (5-bromo-4-chloro-3-indolyphosphate) [sigma] at concentrations of 0.33 mg/ml and 0.17 mg/ml respectively in 5 ml of AP buffer.

For the far-western blots, after the blocking step, GST-UBP was incubated with the blot in I% milk protein in TBS overnight at 4° C. with gentle rocking. Subsequently, the far-western was developed by incubation with anti-GST mouse monoclonal antibody followed by incubation with alkaline phosphatase conjugated goat anti-mouse IgG (Angletti and Engler, 1996, J. Virol. 70:3060-3067; Angeletti and Engler, 1998, J. Virol. 72:2896-2904).

Yeast UBP Gene Disruption

The diploid yeast strain DG401 which was used in gene-knockout experiments was the gift of David Brow (UW-Madison). DG401a has the following genotype: mat a/α, ura3-52/ura3-52, his3-200/his3-200, lys2-801/lys2-801, trp1-901/trp1-901, ade2-101/ade2-101, met⁻/met⁻, gal4-542/gal4-542, gal80-538/gal180-538. UBP gene disruptions were carried out essentially as described (Rothstein, 1991, Methods Enzymol. 194:281-301). The targeting construct was excised from pRS316-His-y-UBP with a Bam HI digest. Approximately 200 ng of the linear fragment was recovered and directly transformed into 10⁵ yeast cells using the standard LiAc method (Schiestl et al., 1993, A Companion to Methods in Enzymology 5:79-85). Transformnants were plated on media lacking His and colonies were isolated after 2-3 days. Correct integrants were identified by PCR amplification using a primer in the chromosomal region upstream of UBP (5′-CTAATCACAACACTTAGC-3′, SEQ ID NO:8) and a primer internal to the His biosynthetic gene (5′-ACTAGAGGAGGCCAAGAG-3′, SEQ ID NO:9). Correct integrants gave rise to a 1.4 kb PCR product, whereas non-homologous integrants gave rise to no product. Positive integrants were subjected to tetrad analysis. To induce to sporulation, the diploid strain was grown in nitrogen-deficient sporulation media and the asci were analyzed for viability by tetrad analysis. Haploid sporulates were then analyzed for the presence of UBP disruption by PCR.

Heat Shock Analysis of y-UBP

Haploid yeast, either wild type or those containing a UBP deletion, ere grown in liquid culture in YPD to a density of 0.5 at OD₆₀₀. The yeast were then subjected to heat treatment at 55° C. for 1 hour. As a control, a duplicate aliquot of cells remained untreated. The yeast were then plated on YPD at dilutions appropriate for counting colonies. After 3 days colonies were counted and the data were expressed as the percent total survivors after heat treatment normalized to wild type.

Hsc70 ATPase Assays

ATPase assays were performed essentially as described (Liberek et al., 1991, J. Biol. Chem. 266:14491-14496). In a reaction volume of 25 μl, pure Hsc70 at a final concentration of 28 nM was incubated with 1 μCi (13 nM) [α-³²P]ATP (Amersham) and various amounts of pure UBP or BSA. The reactions were carried out at 30° C. in ATPase buffer (30 mM 4-(-2 hydroxymethl)-1-piperazineethanesulfonic acid (HEPES) buffer pH 7.5, 40 mM KCl, 50 mM NaCl, 5 mM MgCl₂, and 2 mM dithiothreitol). For the dose curve experiment (FIG. 15A), 0, 12, 58, 118 or 176 nM BSA or UBP were incubated with hsc70 for 1 hour. A 5 μl aliquot of each reaction was directly loaded onto a polyethylamine-cellulose (PEI)-thin layer sheet (Selecto Scientific, Suwanee, Ga.). The products were separated from the substrate by chromatography using 1 M formic acid/1 M LiCl (1:1, vol/vol) as a vehicle. The location of ADP and ATP on the plates was visualized by use of a Phosphorimager (Molecular Dynamics, San Jose, Calif.). The data were quantified and expressed as the percent total hsc70 ATPase activity normalized to the control reactions lacking either BSA or UBP. In the time course experiment (FIG. 15B), hsc70 (28 nM) was incubated with UBP at a final concentration of 28 nM. Five μl aliquots of each reaction were taken at time points of 0, 15, 30, 45, 60, 75 minutes. The samples were analyzed as previously described. Data were quantified and expressed as the percent ATP hydrolysis.

Luciferase Refolding Assays

The assay for the refolding of heat-denatured luciferase was performed as described (Ballinger et al., 1999, Mol. Cell. Biol. 19:4535-3545; Lu and Cyr, 1998, J. Biol. Chem. 273:27S24-27830). Pure luciferase was diluted to 129 nM in refolding buffer (25 mM HEPES, pH 7.4, 50 mM KCl, 5 mM MgCl₂). Luciferase was denatured by incubation at 42° C. for 20 minutes. Refolding reactions contained 28 nM hsc70 and 1 mM ATP in the presence or the absence of pure UBP at a final concentration of 28 mM. Two μl of denatured luciferase was added to each reaction and incubations were carried out for 0 to 100 minutes at 30° C. Samples were analyzed for luciferase activity using a Monolight luminometer (Analytical Luminescence Laboratory, Ann Arbor, Mich.). Data were compiled and graphed using Microsoft Excel.

GST UBP/Hsc70 Pull Down Assay Glutathione sepharose beads (4B; Pharmacia) were equilibrated in GST binding buffer (100 mM NaCl, 20 mM Tris [pH 7.9], 1 mM EDTA, 5% glycerol, 0.02% NP40+1 mM PMSF). Either GST or GST-UBP protein was incubated with a 100 μl bed volume of beads in a total volume of 300 μl of binding buffer at 4° C. with gentle rocking for 2 hours. The beads were washed and then blocked with 5% BSA in binding buffers. The beads were washed in binding buffer then incubated with 200 ng of hsc70 in binding buffer for 2 hours at 4° C. The beads were again washed with binding buffer then 20 μl of beads were added to individual tubes. Each tube was washed 3 times with binding buffer containing 100, 200, 300, or 500 mM NaCl. The beads were resuspended in 1× Laemmli buffer. The samples were heated to 80° C. for 3 minutes then supernatants recovered by centrifugation were analyzed by western blot for hsc70.

Anti-UBP and Anti-hs70 Co-Immunoprecipitation

HeLa cells (5×10⁵/plate) were transfected with 1 μg of Vpu⁺ (MSMBA) or Vpu⁻ (DF101) proviral genomes by calcium phosphate precipitation. A 100 μl bed volume of protein-A sepharose beads (CL4B; Pharmacia) were mixed with either anti-UBP or anti hs70 antibodies in a volume of 300 μl of 1×TBS+1 mM PMSF. The antibodies were allowed to bind with gentle rocking for 1 hour at 4° C. Excess antibody was removed by pelleting and washing the beads twice with 1×TBS+1 mM phenylmethylsulfonylfluoride (PMSF; Sigma). Antibody coated beads were then blocked with 5% BSA in TBS+1 mM PMSF for 1 hour at 4° C. then washed thoroughly. A 20 μl bed volume of antibody coated beads were incubated with 50 μg of the described HeLa cell extracts. Lysates were incubated with gentle rocking at 4° C. for 2 hours. The beads were then pelleted at 5000×g in a microfuge for 3 minutes, and washed with RIPA buffer (50 mM Tris [pH 7.5], 300 mM NaCl, 0.1% SDS, 1% Triton-X 100). The content of p24 Gag co-immunoprecipitated with UBP and his70 was determined by p24 antigen capture ELISA described in the following section.

HIV-1 Particle Release and P24 Antigen Capture ELISA Assays

Triplicate plates of 5×10⁵ HeLa cells were transfected with 1 μg of Vpu⁺ (MSMBA) or Vpu⁻ (DF101) proviral genomes and 10 μg of pHIV-UBP, pHIV-UBP-N, pHIV-FTPR or pHIV-TARluc. Thirty-six hrs after transfection, media were harvested and centrifuged at low-speed to remove cellular debris. The cells were washed with 1×PBS, resuspended in 1 ml of PBS, then pelleted at 5000×g in microfuge tubes. The cell pellets and media were treated with lysis buffer and applied to an HIV-1 p24 antigen assay kit following the instructions of the manufacturer (Coulter, Westbrook, Maine). Samples were processed along with positive controls to give quantitative levels of p24 Gag. Data were plotted as a function of the relative p24 units.

HIV Examples

Recently, a protein which binds to Viral protein U was discovered. Viral protein U is a protein encoded by human immunodeficiency virus type 1 (HIV-1) that promotes degradation of the virus,receptor, CD4, and enhances the release of virus particles from cells. This Viral protein U (Vpu) binding protein was named U binding protein (UBP) (Callahan et al., 1998, J. Virol. 72:5189-5197 [published erratum appears in 1998, J. Virol. 72:8461]). It was found that overexpression of UBP in virus-producing cells resulted in a significant reduction in HIV-1 virion release (Callahan et al., 1998, J. Virol. 72:5189-5197 [published erratum appears in 1998, J. Virol. 72:8461]). It was also found that UBP interacts directly with HIV-1 Gag protein. UBP is a member of the tetratricopeptide repeat (TPR) co-chaperone protein family containing four copies of the 34-amino acid TPR motif. The ubp gene is highly conserved evolutionarily and is ubiquitously expressed in human tissues. SGT (“small glutamine-rich protein”; Genbank Accession: XM_{—}009137), is identical to UBP (Bankit416179) and was independently identified as a rat cellular protein that interacts with the nonstructural protein, NS-1 from autonomous Parvovirus H-1 (Cziepluch et al., 1998, J. Virol. 72:4149-4156). NS-1 is required for viral DNA replication and is found together with SGT in nuclear foci that are the site of H-1 DNA synthesis (Cziepluch et al., 2000, J. Virol. 74:4807-4815).

To elucidate the normal role of UBP in cells, and to understand how Vpu-UBP and UBP-Gag interaction is related to HIV particle exit, it was attempted to identify other cellular proteins that stably interact with UBP. To this end, HeLa cell lysates were subjected to “far-western” analysis. This entailed separation of the cell proteins on SDS gels and electrophoretic transfer to an Immobilon P membrane. The membrane was then incubated with GST-UBP and stable association of the UBP with transferred protein was detected using anti-GST antibody as in conventional western analysis. Data from this experiment indicated that a protein of about 70 kD was the primary peptide that stably interacted with UBP (FIG. 13A).

Using a yeast two hybrid screen for proteins that interact with hsp70, Liu et al. found that hs70 (referring to both hsp70 and hsc70) interacts with UBP (Liu et al., 1999, J. Biol. Chem. 274:34425-34432). Moreover, UBP contains TPR motifs in an array similar to that found in co-chaperones such as Hip, Hop, and Chip (Ballinger et al., 1999, Mol. Cell. Biol. 19:4535-4545; Chen and Smith, 1998, J. Biol. Chem. 272:35194-35200; Ha and McKay, 1995, Biochemistry 34:11635-11644). Thus, purified hsp70 and hsc70 were used in far-western analyses in parallel with HeLa cell proteins to see whether the 70 kD protein was hs70. This indicated that UBP does stably associate with both hsp70 and hsc70 in this in vitro assay (FIG. 13B). To examine the stability of this UBP-hs70 interaction, the protein complexes were subjected to washes of increasing NaCl concentrations. This indicated that the protein was maintained even in the presence of relatively high salt concentrations (0.5 M NaCl) and is consistent with a robust interaction between UBP and hs70 (FIG. 13C).

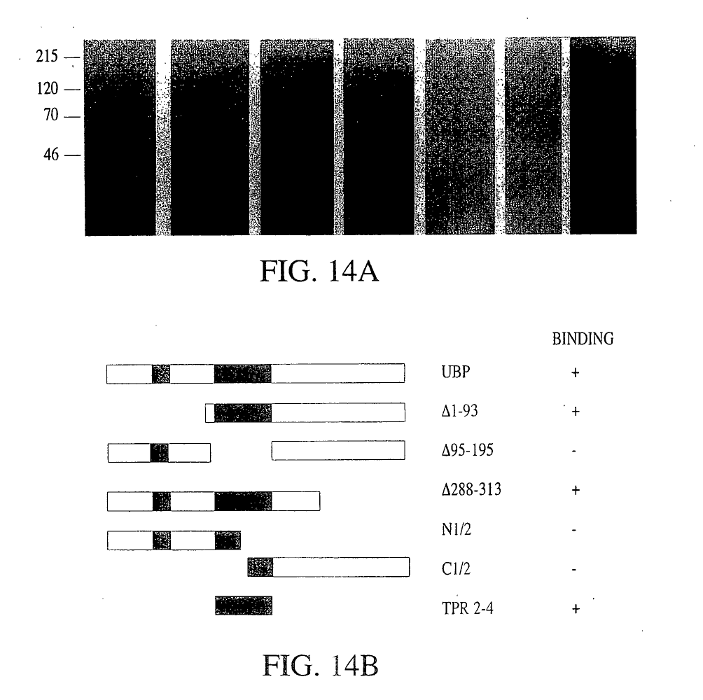

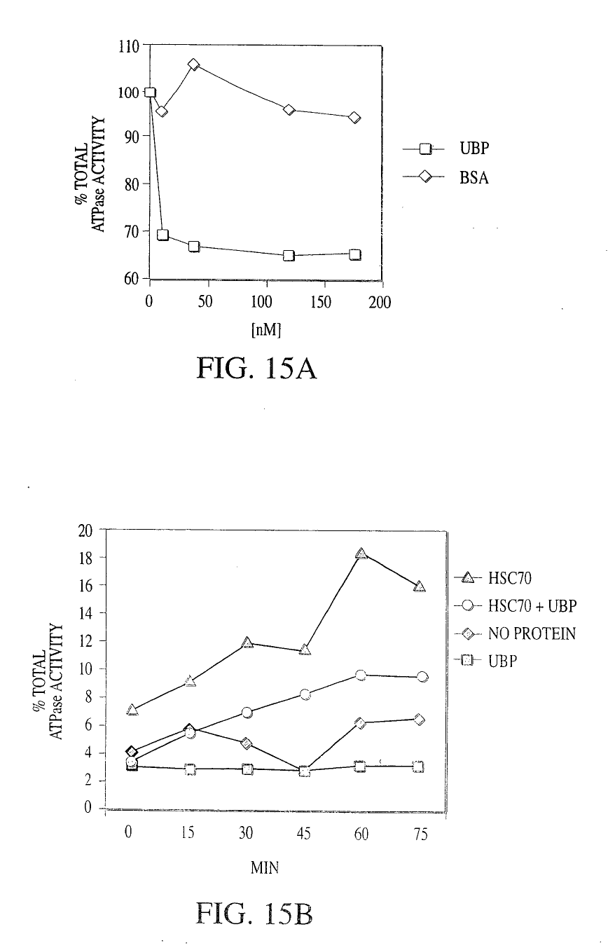

Several co-chaperones that mediate the activity of hs70 contain TPR motifs and interact with hs70 by way of their TPR motifs. TPRs are generally considered to be motifs that mediate intermolecular interaction by way of a signature alpha helix (Das et al., 1998, EMBO J. 17:1192-1199). Thus, the hypothesis tested next was that the TPRs in the N-terminal half of UBP are required for U BP-Hs70 interaction. Several deletion mutants of UBP were constructed that lack various segments of the protein and these were tested for interaction with hsc70 using far-western analysis (FIG. 14). These data indicate that the TPRs of UBP are indeed necessary for interaction with hs70. UBP mutants that maintained the three tandem TPRs (Δ1-93, Δ288-313, and TPR 2-4) were capable of stable interaction with hsc70 while those that lacked the three TPRs (Δ95-195, N 1/2, and C 1/2) did not detectably interact with hsc70. Further, the fact that N 1/2 and C/2, both of which contain a single intact TPR, were unable to associate with hs70, indicates that a single TPR is unlikely to be sufficient for association with hsc70. TPR 2-4 is a mutant that expresses only the three tandem TPRs (a.a. 95-195). Since this mutant was able to stably interact with hsc70, it appears that these three contiguous TPRs (a.a. 95-195) are sufficient for UBP-hs70 interaction. However, in additional experiments this interaction was found to enhance when a fragment containing TPR 2-4 as well as flanking charged residues (a.a. 81-209).

Hs70 contains an intrinsic ATPase activity that is important in the activity and function of the multiprotein chaperone complex. The ATP-bound form of hs70 has relatively low affinity for protein substrates whereas the ADP-bound form of hs70 has relatively high affinity for protein substrates. Regulatory co-chaperones that associate with hs70, such as hsp40, BAG-1, Hip and Chip, often exert their effect by positively or negatively affecting the ATPase activity of Hs70 (Ballinger et al., 1999, Mol. Cell. Biol. 19:4535-4545; Bimston et al., 1998, EMBO J. 17:6871-6878; Ha and McKay, 1995, Biochemistry 34:11635-11644). To determine whether UBP might similarly affect the ATPase activity of hsc70, in vitro ATPase assays were carried out in the presence of UBP. This analysis indicated that UBP negatively affected the hsc70-mediated hydrolysis of ATP (FIG. 15). The magnitude of this effect was similar to that of the co-chaperone Chip, a co-chaperone that negatively affects the ATPase activity of hs70. These data are consistent with the idea that UBP is also a co-chaperone that affects the activity of hs70.

Although hs70 functions as part of a multiprotein complex, the protein is able to independently promote the refolding of denatured protein in vitro in the presence of ATP. To see whether UBP can affect this refolding activity of hs70 we carried out an assay to detect the refolding of denatured luciferase in the presence and absence of UBP (FIG. 16). As expected, hsc70 was able to catalyze the refolding of heat-denatured luciferase to functional form. UBP inhibited the hs70-dependent refolding of luciferase by about 30%. This is consistent with the observed negative effect of UBP on the ATPase activity of hsc70, similar in effect and magnitude to Chip's affect on hsc70-mediated protein refolding, and again indicative of a likely role for UBP as a co-chaperone. Furthermore, the data described in FIG. 15 and in FIG. 16 demonstrate that the magnitude of UBP inhibition of hsc70 ATPase activity exactly mirrors the UBP-dependent inhibition of hsc70 refolding activity.

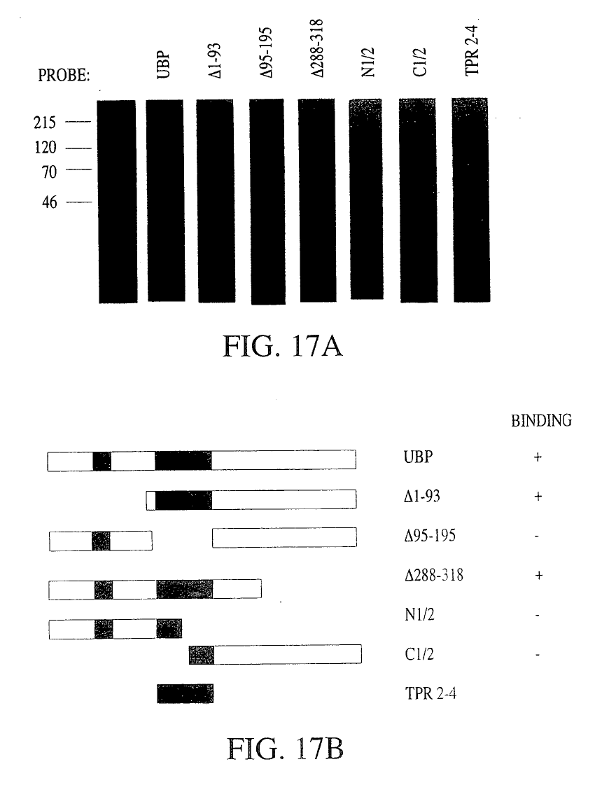

Hsp90 is another key protein that is found in the foldsome and works in conjunction with other members of the chaperone complex to facilitate correct substrate structure. Some TPR-containing co-chaperones, such as Hip and Hop, are able to interact with both hs70 and hsp90 by way of their TPRs (Johnson et al., 1998, J. Biol. Chem. 273:3679-3686). Additional hsp90-associated proteins such as the peptidylprolyl isomerases, Cyp40 and FI(BP52, and the protein phosphatase PP5, also interact with hsp90 by way of TPR motifs (Das et al., 1998, EMBO J. 17:1192-1199; Ratajczak and Carrello, 1996, J. Biol. Chem. 271:2961-2965). In the far-western analysis, it was noticed that UBP was able to associate with a 90 kD protein from HeLa cells (indicated with an asterisk in FIG. 13B), albeit the signal was weaker compared to that for hs70. To investigate this further, purified hsp90 was analyzed using far-western analysis with UBP as a probe. Based on this experiment, it appeared that UBP could associate with hsp90 in vitro (FIG. 17). The ability of the various UBP deletion mutants to stably interact with hsp90 was then evaluated. This analysis revealed that the three tandem TPRs of UBP were necessary for interaction between UBP and hsp90. However, in contrast to UBP-hs70, the interaction of hsp90 with the three TPRs was too weak to detect suggesting that the specificity provided by the flanking charged amino acids was required for this interaction.

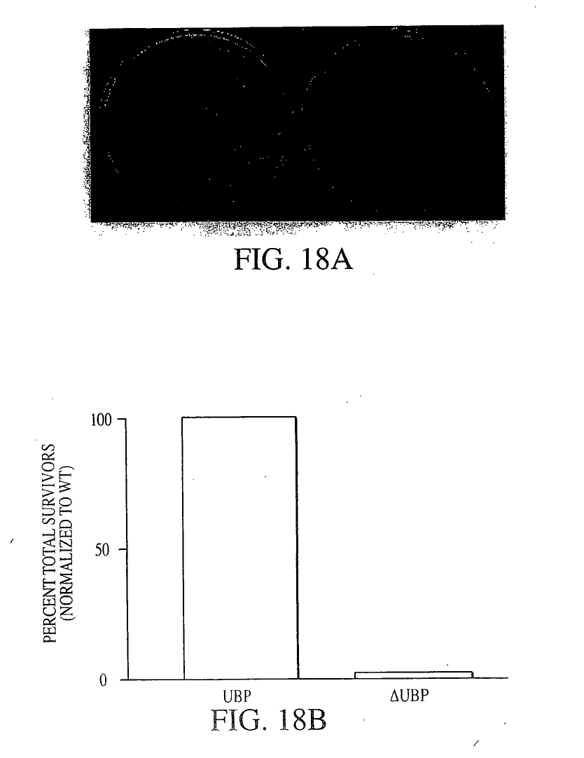

UBP appears to be a highly conserved gene, and diverse organisms including D. melanogaster, C. elegans, and S. cerevisiae, contain apparent homologs to human UBP (Callahan et al., 1998, J. Virol. 72:5189-5197 [published erratum appears in 1998, J. Virol. 72:8461]; Cziepluch et al., 1998, J. Virol. 72:4149-4156). To determine whether UBP may function as a co-chaperone in yeast, homologous recombination was used to generate a knockout strain containing a deletion in the yeast UBP gene (y-UBP). This y-UBP mutant was viable when grown on rich medium and at 37° C. Hsp70 and hsp90 are members of a large class of “heat shock” proteins that were originally identified by increased expression following heat treatment. The increased expression of many of these heat shock proteins results in a corresponding increase in chaperone activity and concomitant refolding of heat-denatured proteins. Not Surprisingly, many yeast strains that are deficient in appropriate co-chaperone activity and their regulation, are also deficient in recovery from various heat shock treatments. The ability of the y-UBP null mutant to recover from heat shock was then tested. The thermotolerance of the mutant was indistinguishable from that of wild type when the cells were incubated at 18° C., 32° C., and 42° C. However, the y-UBP deletion mutant exhibited a marked reduction in viability following a high temperature (55° C.) heat shock (FIG. 18). The viability of the mutant was reduced by at least 50 fold relative to wild type by this treatment. Such a phenotype is similar to that observed for some yeast strains containing lesions in genes encoding protein chaperone functions, such as the gene HSP104 (Sanchez and Lindquist, 1990, Science 248:1112-1115). Thus, the phenotype of the y-UBP knockout mutant is consistent with a role for UBP as a functional component of the protein chaperone complex in yeast.

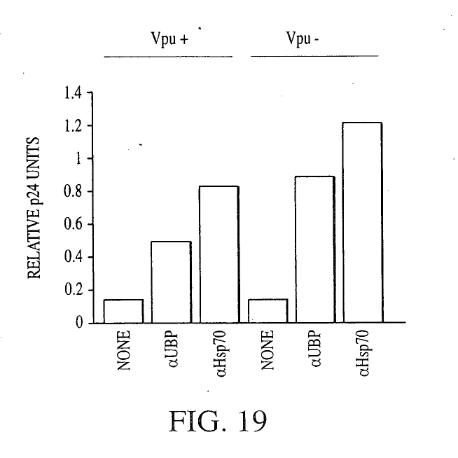

Given these observations, which indicated that UBP is likely to be a co-chaperone, it is possible that Vpu promotes HIV particle exit by way of interaction with UBP and modulation of the protein chaperone complex. The principal structural protein of the viral capsid, Gag, also interacts with UBP. Thus, it seemed likely that both Gag and Vpu would be associated with the protein chaperone complex by way of UBP. Co-immunoprecipitation experiments were performed to determine whether Gag was associated with the chaperone complex in the presence and absence of Vpu, and to see whether Vpu expression alters the in vivo association of UBP and Gag. HeLa cells were transfected in parallel with virus expression constructs that either express wild type Vpu or that are null for Vpu. Hs70 or UBP were then precipitated using anti-hs70 or anti-UBP antibody, and associated Gag protein was assayed using an antigen capture assay specific for p24, the major capsid protein derived from the Gag gene (FIG. 19). This experiment indicated that Gag was associated with hs70, consistent with the hypothesis that Gag is indirectly or directly associated with the protein chaperone complex by way of UBP. In addition, the association of Gag with both UBP and hs70 appeared not to be affected by simultaneous expression of Vpu.

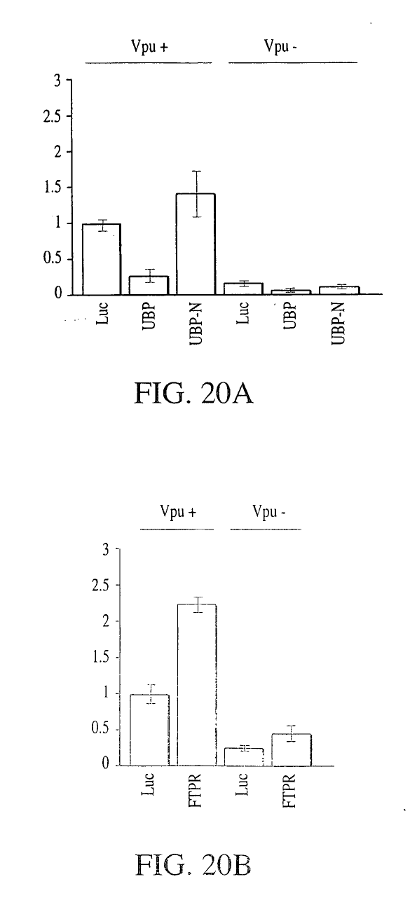

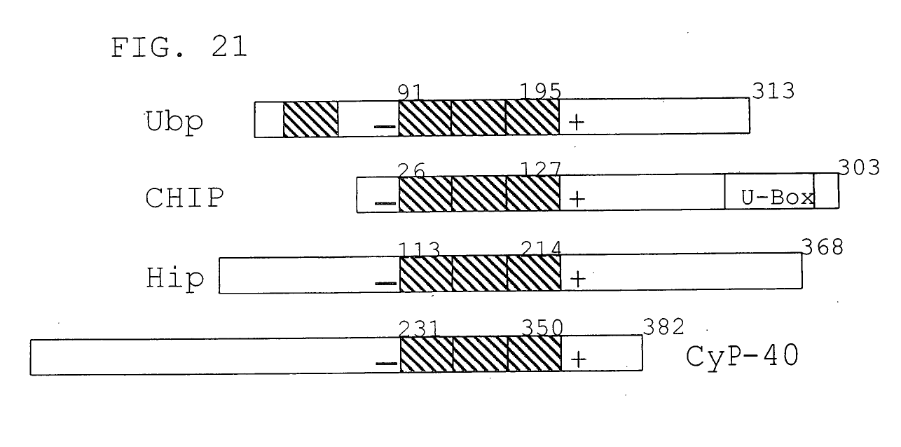

Overexpression of UBP in virus-producing cells was previously shown to have a negative effect on particle exit (Callahan et al., 1998, J. Virol. 72:5189-5197 [published erratum appears in 1998, J. Virol. 72:8461]). Since the three tandem TPRs of UBP appeared to be necessary and sufficient for interaction with hs70 in the far-western assay, it was determined whether high level expression of a UBP fragment containing the three TPRs might dominantly interfere with UBP-hs70 interaction and thereby affect particle exit. Expression constructs were generated in which either the three TPRs (pHIV-FTPR), or the entire N-terminal portion of UBP (pHIV-Ubp-N), were expressed from the HIV-1 promoter. These constructs were then co-transfected into cells along with virus expression plasmids that either contained or lack Vpu. As a control, cells were co-transfected with a construct that expresses the luciferase gene under the control of the HIV LTR (pHIV-TARluc). The efficiency of virus particle exit was then measured; where the efficiency of particle release is given by the ratio of extracellular to intracellular Gag. Vpu promotes efficient particle exit from cells and this is exemplified by comparison of the extracellular/intracellular p24 following cotransfection with pHIV-TARluc in the presence and absence of Vpu (FIG. 20A and FIG. 13B). As expected, based on previous observations, co-expression of full length UBP resulted in depression of particle exit (FIG. 20A). Interestingly, co-expression with either pHIV-Ubp-N (FIG. 20A) or pHIV-FTPR (FIG. 20B) resulted in a slight increase in the efficiency of virus particles when Vpu was present. However in the absence of Vpu, co-expression of either Ubp-N or FTPR did not significantly increase particle exit.

FIG. 21 provides a summary of the constructs of several TPR-containing co-chaperones used in the HIV studies. These include the Ubp, CHIP, Hip, and CyP-40 constructs.

The disclosures of each and every patent, patent application, and publication cited herein are hereby incorporated herein by reference in their entirety.

While this invention has been disclosed with reference to specific embodiments, it is apparent that other embodiments and variations of this invention may be devised by others skilled in the art without departing from the true spirit and scope of the invention. The appended claims are intended to be construed to include all such embodiments and equivalent variations.

1. A method of inhibiting virus replication in a cell wherein a heat shock protein is required for replication of said virus, said method comprising administering to said cell a virus replication-inhibiting amount of a heat shock protein inhibitor, thereby inhibiting virus replication in said cell.

2. The method of claim 1, wherein said cell is an avian cell.

3. The method of claim 1, wherein said cell is a mammalian cell.

4. The method of claim 3, wherein said mammalian cell is a human cell.

5. The method of claim 1, wherein said heat shock protein is selected from the group consisting of a heat shock protein 27, a heat shock protein 40, a heat shock protein 70, and a heat shock protein 90α.

6. The method of claim 5, wherein said heat shock protein is heat shock protein 40.

7. The method of claim 1, wherein said heat shock protein inhibitor inhibits a heat shock protein interaction required for virus replication.

8. The method of claim 7, wherein said heat shock protein inhibitor inhibits interaction of a heat shock protein 40 with a heat shock protein 70.

9. The method of claim 8, wherein said heat shock protein inhibitor is a peptide comprising a heat shock protein 40 J domain comprising SEQ ID NO:1.

10. The method of claim 8, wherein said heat shock protein inhibitor is a synthetic peptide comprising a heat shock protein 40 J domain.

11. The method of claim 9, wherein said heat shock protein 40 J domain comprises from about amino acid I to amino acid 70 of SEQ ID NO:1.

12. The method of claim 9, said method comprising administering an isolated nucleic acid encoding said heat shock protein 40 J domain, wherein when said nucleic acid is expressed in said cell said heat shock protein 40 J domain inhibits interaction of a heat shock protein 40 with a heat shock protein 70.

13. The method of claim 1, wherein said virus is selected from the group consisting of a papillomavirus, a cytomegalovirus, a measles virus, a Newcastle's disease virus, a respiratory syncitial virus, a herpes simplex virus, a human immunodeficiency virus 1, a hantavirus and an adenovirus.

14. The method of claim 13, wherein said adenovirus is chicken embryo lethal orphan (CELO) virus.

15. The method of claim 1, wherein said heat shock protein inhibitor is selected from the group consisting of an isolated nucleic acid, an expression vector, an antisense nucleic acid, a protein, a peptide, an antibody, a transcription inhibitor, a translation inhibitor, and an antiviral agent.

16. A method of inhibiting virus replication in an animal wherein a heat shock protein is required for said virus replication, said method comprising administering to said animal a virus replication-inhibiting amount of a heat shock protein inhibitor, thereby inhibiting virus replication in said animal.

17. The method of claim 16, wherein said heat shock protein required for said virus replication is selected from the group consisting of a heat shock protein 27, a heat shock protein 40, a heat shock protein 70, and a heat shock protein 90α.

18. The method of claim 17, wherein said heat shock protein is a heat shock protein 40.

19. The method of claim 16, wherein said heat shock protein inhibitor inhibits interaction of a heat shock protein 40 with a heat shock protein 70.

20. The method of claim 19, wherein said heat shock protein inhibitor is a peptide comprising a heat shock protein 40 J domain.

21. The method of claim 20, wherein said heat shock protein 40 J domain comprises from about amino acid 1 to amino acid 70 of SEQ ID NO:1.

22. A kit for inhibiting virus replication in a cell wherein a heat shock protein is required for said virus replication, said kit comprising a heat shock protein inhibitor, an applicator, and an instructional material for the use thereof.

23. The kit of claim 22, wherein said heat shock protein inhibitor is selected from the group consisting of a peptide comprising a heat shock protein 40 J domain, a nucleic acid encoding a heat shock protein 40 J domain, a nucleic acid complementary with a nucleic acid encoding a heat shock protein 40 J domain wherein said nucleic acid is in an antisense orientation, and an antibody that specifically binds with a heat shock protein 40 wherein when said antibody binds with said hsp40 binding of said hsp40 with hsp70 is inhibited.

24. A kit for inhibiting virus replication in an animal infected with a virus wherein a heat shock protein is required for said virus replication, said kit comprising a heat shock protein inhibitor, an applicator, and an instructional material for the use thereof.

25. A method of inhibiting virus replication in a cell wherein a heat shock protein is required for replication of said virus, said method comprising administering to said cell a virus replication-inhibiting amount of a flavonoid, thereby inhibiting virus replication in said cell.

26. The method of claim 25, wherein said flavonoid is selected from the group consisting of naringenin, naringin, morin, catechin, kaempferol, myricetin, phloretin, phlorizdin, rutin, 3-methylquercetin, and quercetin.

27. The method of claim 26, wherein said flavonoid is quercetin.

28. The method of claim 25, wherein said virus is selected from the group consisting of a papillomavirus, a cytomegalovirus, a measles virus, a Newcastle's disease virus, a respiratory syncitial virus, a herpes simplex virus, a human immunodeficiency virus 1, a hantavirus and an adenovirus.

29. The method of claim 28, wherein said virus is hantavirus.

30. The method of claim 29, wherein said virus is Sin Nombre hantavirus.

31. An isolated nucleic acid complementary to a nucleic acid encoding a heat shock protein, or a fragment thereof, said complementary nucleic acid being in an antisense orientation.

32. A vector comprising the isolated nucleic acid of claim 31.

33. A composition comprising the isolated nucleic acid of claim 31, and a pharmaceutically-acceptable carrier.

34. A non-human transgenic mammal comprising the isolated nucleic acid of claim 31.

35. A method of inhibiting virus replication in a cell wherein a heat shock protein is required for replication of said virus, said method comprising administering to said cell a virus replication-inhibiting amount of an isolated nucleic acid complementary to a nucleic acid encoding a heat shock protein, or a fragment thereof, said complementary nucleic acid being in an antisense orientation, thereby inhibiting virus replication in said cell.

36. The method of claim 35, wherein said heat shock protein is selected from the group consisting of heat shock protein 27, heat shock protein 40, heat shock protein 70, heat shock protein 72 and heat shock protein 90.

37. A method of treating a virus related disease in an animal wherein a heat shock protein is required for replication of said virus, said method comprising administering to said animal a virus replication-inhibiting amount of a composition comprising an inhibitor of heat shock protein dependent virus replication, said composition further comprising a pharmaceutically-acceptable carrier, thereby treating said virus related disease.

38. The method of claim 37, wherein said inhibitor is a flavonoid.

39. The method of claim 38, wherein said inhibitor is quercetin.

40. The method of claim 37, wherein said inhibitor is an isolated nucleic acid complementary to a nucleic acid encoding a heat shock protein, or a fragment thereof, said complementary nucleic acid being in an antisense orientation.

41. A method of inhibiting heat shock protein dependent virus replication in a cell wherein a heat shock protein is required for replication of said virus, further wherein said virus is hum immunodeficiency virus-1 (HIV-1), said method comprising administering to said cell a virus replication-inhibiting amount of a heat shock protein inhibitor, thereby inhibiting virus replication in said cell.

42. The method of claim 41, wherein said heat shock protein inhibitor comprises viral particle u binding protein (UBP), or a derivative or fragment thereof.

43. The method of claim 41, wherein said heat shock protein inhibitor inhibits a heat shock protein interaction required for virus replication.

44. The method of claim 41, wherein said beat shock protein inhibitor inhibits a heat shock protein function selected from the group consisting of heat shock protein ATPase activity and heat shock protein folding function activity.

45. A non-human transgenic mammal comprising an isolated nucleic acid encoding a viral particle u binding protein (UBP), or a derivative or fragment thereof.

46. A non-human transgenic mammal comprising an isolated nucleic acid encoding an inhibitor of heat shock protein dependent virus replication.

47. A method of inhibiting virus replication in a cell wherein a heat shock protein is required for replication of said virus, said method comprising administering to said cell a virus replication-inhibiting amount of an isolated nucleic acid encoding viral particle u binding protein (UBP) or derivatives or fragments thereof, further wherein when said nucleic acid is expressed in said cell, said UBP protein, derivatives or fragment thereof inhibit a heat shock protein, thereby inhibiting virus replication in said cell.

48. The method of claim 47, wherein said heat shock protein is heat shock protein 70.

49. The method of claim 47, wherein said heat shock protein is heat shock protein 90.

50. A method of treating a virus related disease or disorder in an animal wherein a heat shock protein is required for replication of said virus, said method comprising administering to said animal a virus replication-inhibiting amount of a composition comprising an isolated nucleic acid encoding viral particle u binding protein (UBP) or derivatives or fragments thereof, said composition further comprising a pharmaceutically-acceptable carrier, thereby treating said virus related disease.

51. A method of identifying a compound which inhibits heat shock protein dependent virus replication, said method comprising:
a. contacting a cell with a test compound;
b. comparing the level of heat shock protein function in said cell with the level of heat shock protein function in an otherwise identical cell not contacted with said test compound, wherein a lower level of said heat shock protein function in said cell contacted with said test compound compared with the level of heat shock protein function in said otherwise identical cell not contacted with said test compound is an indication that said test compound inhibits heat shock protein function;
c. when said test compound inhibits heat shock protein function, adding said test compound to a virus-infected cell and comparing the level of virus replication in said cell with the level of virus replication in an otherwise identical cell not contacted with said test compound, wherein a lower level of said virus replication in said virus-infected cell contacted with said test compound compared with the level of virus replication in said otherwise identical cell not contacted with said test compound is an indication that said test compound inhibits virus replication;
d. thereby identifying a compound which inhibits heat shock protein dependent virus replication.

52. The method of claim 51, wherein said compound inhibits a heat shock protein selected from the group consisting heat shock protein 27, heat shock protein 40, heat shock protein 70, and heat shock protein 90.

53. The method of claim 52, wherein said compound inhibits heat shock protein 40.

54. The method of claim 52, wherein said compound inhibits heat shock protein 70.

55. The method of claim 52, wherein said compound inhibits heat shock protein 90.

56. The method of claim 51, wherein said virus is selected from the group consisting of a papillomavirus, a cytomegalovirus, a measles virus, a Newcastle's disease virus, a respiratory syncitial virus, a herpes simplex virus, a human immunodeficiency virus 1, a hantavirus and an adenovirus

57. The method of claim 56, wherein said virus is selected from the group consisting of adenovirus, hantavirus, and human immunodeficiency virus-1.

58. The method of claim 51, wherein said heat shock protein function is a heat shock protein interaction.

59. The method of claim 51, wherein said heat shock protein function is ATPase activity.

60. The method of claim 51, wherein said heat shock protein function is folding activity.

61. The method of claim 51, wherein said cell is an avian cell.

62. The method of claim 51, wherein said cell is a mammalian cell.

63. The method of claim 62, wherein said mammalian cell is a human cell.

64. The method of claim 51, wherein when said heat shock protein function is a heat shock protein interaction, said method further comprises contacting a cell with a test compound and comparing the level of interaction of a first heat shock protein with a second heat shock protein in said cell contacted with said test compound with the level of interaction of said first heat shock protein with said second heat shock protein in an otherwise identical cell not contacted with said test compound, wherein a lower level of said interaction of said first heat shock protein with said second heat shock protein in said cell contacted with said test compound compared with said level of interaction of said first heat shock protein with said second heat shock protein in said otherwise identical cell not contacted with said test compound is an indication that said test compound inhibits a heat shock protein interaction.

65. The method of claim 64, wherein said first heat shock protein is selected from the group consisting of a heat shock protein 27, a heat shock protein 40, a heat shock protein 70, and a heat shock protein 90α.

66. A compound identified by the method of claim 64.


SEQUENCE LISTING (9 sequences)

SEQ ID NO: 1 - protein, 70 aa, Homo sapiens
MGKDYYQTLGLARGASDEEIKRAYRRQALRYHPDKNKEPGAEEKFKEIAEAYDVLSDPRKREIFDRYGEE

SEQ ID NO: 2 - protein, 340 aa, Homo sapiens
MGKDYYQTLGLARGASDEEIKRAYRRQALRYHPDKNKEPGAEEKFKEIAEAYDVLSDPRKREIFDRYGEEGLKGSGPSGGSGGGANGTSFSYTFHGDPHAMFAEFFGGRNPFDTFFGQRNGEEGMDIDDPFSGFPMGMGGFTNVNFGRSRSAQEPARKKQDPPVTHDLRVSLEEIYSGCTKKMKISHKRLNPDGKSIRNEDKILTIEVKKGWKEGTKITFPKEGDQTSNNIPADIVFVLKDKPHNIFKRDGSDVIYPARISLREALCGCTVNVPTLDGRTIPVVFKDVIRPGMRRKVPGEGLPLPKTPEKRGDLIIEFEVIFPERIPQTSRTVLEQVLPI

SEQ ID NO: 3 - DNA, 7246 bp, Homo sapiens
agatctgcctgctggatgctcctgatagttatttatgtaactatccgccatgatgctcctgatagttatttatgtaacttttttatggggaggctgagggtctcactgtcacccaggctggagtgcagtggcacaatcatagctgcctgcagcctcaaattcctgggctcaagagatcctcccgcctcaggaggcaggaggatggtcttcttcttcaggagtccctgtctgcccaggggcagctgtcactggaatcttttagctgccaggaatgaggtgatggctgaagagcccaccactaagaaaatggttagtcacccctttgacaatgcttcctttgtgccatgcctgctctgagcactttgcaaatgttgactcatataaacttcacagccctcatgatgtgggcacaactgctatcctcattcctcattgtgcagatgaggaagctaaggccagcaaaggcctagtaactaacccaaggttacccagccgggaaatgctggagctgggaatgaagcccagctgtctggttccagagccagtgttacatggctctgacctcccaggcagttcttccaggaacctcccccacttccctgggcccttcccaggtggaacccctttctgagcctccttttcccatcagctctgtccttgacagaggcaccccagactttcagagccaaacgcgatcgtgttgcctacaattgaggaaacagaggccttgaaaggtgtgtgacacacctatgtccccccaaattataaaggtagttggggaagaaaaagaagcacgtgtgtaagaacggggaatatttatgaggttctcactgcgttccaggcactggggacccagtgcagacaggacctggcccctgcccaggtggcgatgtcagactgggagggaagacacattgaaagccaagagacaagataaatttcaaatttagcggcaagtgatcttgagagggcagtaaagggaagaaggcgggactgggaggaccttgggagatgcgcttccgtccagatcctcaccttgctttaggaggtgagaacagaggcagtcttccgtcccaaaatccttctccccacggttgggagaatagcgctggtgggttttttgttcattttactttattttacttattttatttatttatgagacggagtctcgctctgttgccgaggctggagtgcaacggccggatctcggctcactgcaacctccgcctccccgattcaagctattctcaggcctcaatctcctgagtagctgggattacaggttcgtgccaccaacgcccgggtaatttttatatttttagcagagatggggtttcaccatgttggccaggctggtctcgaactcctgacctcaagtgatccgcccgcctcggcctcccaaaatgctgggattacaggcgtgagccaccgcgcccggcctgtttgttaatcttaaatagaaacggagtctccttatgttgcctaggctggtctccaactcctaggatcaagagatcctcctgtcttggcctcaggaagtgatgggattacaggcatgaggctccgcgcccggcctgataggttctcttattgccatgttgtagatagaagggggcccctgggttaggcagacacaggttaggtagttcgtccggcgtcaccgggcgggagcccaccgcaggccccacgagacaagggcgagcaggttcatcgcctcccggggaaaggactgggtagtcccaggccccaccccttcctggaacagcaagttcgctgtgggtcccgacctactgacccaggcggagggcgggacgcagggtagctggggccgcgtagagagggaaagaaggtcgcgattggctcgtcccgaaaggtgggcggggcctcctccgacctgtgcgcgcgcgccgcggggaggtgttgccgagggcggagcgggagggggcgtggccccgcgcgggcggccgttgacggcgaggccccgccccggatgtcgcgtgtcgctgaaagggcggcggcgattggccggcgccgcgggggcgggcggggcggaaggttctggagggggctggcgggctctggaagcttccgccggacgggtatatagagtccgggactggtcggcggcggagccgggggacggcgacagcgggtcggcgggccgcaggagggggtcatgggtaaagactactaccagacgttgggcctggcccgcggcgcgtcggacgaggagatcaagcgggcctaccgccgccaggcgctgcgctaccacccggacaagaacaaggagcccggcgccgaggagaagttcaaggagatcgctgaggcctacgacgtgctcagcgacccgcgcaagcgcgagatcttcgaccgctacggggaggaaggtgtgtgctcgcggccaggggcgcggccccgccttttgacagacagggaaactgaggcacgctggcccctctcggcggccccccgggaaggacgccccgggccgtggggccgagctgccccgccctccggtctcgcgggcctccgccctcggattggcggccgcgcggtgggaggaggagccttggcccagccgctcggccagaagcttctagcatgtctggggctgcccctccccgggcgcctcctcccgcggccccgagcagccccggggtgcggtgcatgggggtgggggagccgggggtgacgacggggacggcgcggcggagcccgctgcggacccgggctcacctgggctcggccgccggggtccgcggggcggcgcctccggctcagctgcggggcgaggggttgtgaatgcaggagacgaccccgttcgtgggcttgggggctgggttgggataattcccgggaagtgatgacctggccccggcagggcacgcaggaggcagcggccgcccaggtccggagagcggggccgcctcggagggtaaggagagcagggctttttgttcctgtgcccgttgatgattcaggctggcttctgggcttgatctggagctgactttttgtgcaaggatggtggcagtgaatgctaatgtgtgaggatttaaaaactccctatttatttttgttttttttaatcttctcttcctacgcaccattagccctaaaacgttgagagtaaacacttaccgaaagccgaccctggaccatgtgtctttagccatctcttggtgatgttgccaggaggatgggtcagtccaggagtggcagggcctaggtggcacctggaggtgaacacctaaccgcatgagtccttttccactgtcaggctacgcggaaatactgttggtaggggggcgtctctgctgttggcagtgagaggtagaaacttggggttcttccttgctctcccctgacagacaaaaaagttcctcccccatggcttccaagctgtttccccctgtaagggaagctcgagagagagtctgctgttcagccatctgactgcttctaaatctgcttttcaggcctaaaggggagtggccccagtggcggtagcggcggtggtgccaatggtacctctttcagctacacattccatggagaccctcatgccatgtttgctgagttcttcggtggcagaaatccctttgacaccttttttgggcagcggaacggggaggaaggcatggacattgatgacccattctctggcttccctatgggcatgggtggcttcaccaacgtgaactttggccgctcccgctctgcccaagagcccgcccgaaagaagcaagatcccccagtcacccacgaccttcgagtctcccttgaagagatctacagcggctgtaccaagaagatgaaaatctcccacaagcggctaaaccccgacggaaagagcattcgaaacgaagacaaaatattgaccatcgaagtgaagaaggggtggaaagaaggaaccaaaatcactttccccaaggaaggagaccagacctccaacaacattccagctgatatcgtctttgttttaaaggacaagccccacaatatctttaagagagatggctctgatgtcatttatcctgccaggatcagcctccgggaggtaaggtgccaggtggggcggtgtctgtagagggcgatggctgctttttgaagcagtttagtatttgctgaaccaatgtgttggggtgtgtggtgcgtgccaggcttggggcacaacagtgaacaggactgaccagggcgctgttgtggagctttcgtggtggggacgtgtagatttgggggccaggtcttagctgcagaggcctgatgggtcttatctatggcagacggcctctgggcaagagctgagcctcttgagccagcttccatctaatgctgtccttttgcttttcaaggctctgtgtggctgcacagtgaacgtccccactctggacggcaggacgatacccgtcgtattcaaagatgttatcaggcctggcatgcggcgaaaagttcctggagaaggcctccccctccccaaaacacccgagaaacgtggggacctcattattgagtttgaagtgatcttccccgaaaggattccccagacatcaagaaccgtacttgagcaggttcttccaatatagctatctgagctccccaaggactgaccagggacctttccagagctcaaggatttctggacctttctaccagttgtggaccatgagagggtgggagggcccagggagggctttcgtactgctgaatgttttccagagcatatattacaatctttcaaagtcgcacactagacttcagtggtttttcgagctatagggcatcaggtggtgggaacagcaggaaaaggcattccagtctgccccactgggtctggcagccctcccgggatgggcccacatccacctccagtccctggccaggggtgagaggcagaccagcagatggacttgatccctctgtgtctttttgcttctggctggtagataatgtcaacctgcagtcttgattcccagaccctgtacactcctccttttctgttgtgtgatcagtttgtgctttattctgtatttgtctcccatgtcttgctcttctcctggagaattctgtcttctctttggccatctcaaattgagaacctaaactattcctgcagaactgcctggttggcgtccacaagcaatacctctcgttccagcaggaccaagggagccagcctccagtgagtgactccagcaagtgcagccacctctcccttgatggtctgggagcctggcctcagcaaggggccttcctgacctctggctccagtgaagctgaatgtcctcactttgtgggtcacactctttacatttctgtaaggcaatcttggcacacgtggggcttaccagtggcccaggtaattttttgtttcatggactatggactctttcaaagggatctgatccttttgaattttgcacagccctagatacaatcccttttgataaaagggtctttgcttctgattacaggagcactgtggaacgtctgtaaatatgtttttataattccatgtatagttggtgtacactcaaaacctgtccccggcagccagtgctctctgtatagggccataatggaattctgaagaaatcttggggagggaaggggagttggaacaaatgtctgttccctggaggccagtccagtgctcagacctttagactcattgtaagttgccactgccaacatgagaccaaagtgtgtgactagtcaatgaagtgcgacagcattaaagactgatgctaaacctcaggggagcggtcctgtgactctgtttgagggttctgctggttttgggggtggagtggagagctgggcatccttccaaattcaatcaaggtgagagggtgggatgggcaggaggcaagtgccctgcaagggaaacgtaagtctcccttcctcagccataagtgggttggagaacttctacccagactcggggtccaaaaaaccagagacccaagcacagtggccttggggatcattttttgatgaaggttggagtcaaagtagcggtggggagaggcagggtctggctcctaattctgcctttcttcagggtccctggctttttcagcagaccttggctctggggccaaggggccaacccagggtctggcctcttaattccttatcactccttccctgctagaacgggctgggcgtcctgaaggctggccaaacccgagctcaaggtcctgcccaggactagccccactgtagagaggacccatttcctgccttggctttatttgcaggctactaaagctgcttttactttgtaactttctttaaaataactggttttattataaagtaattgctcaggcttaaaacaatgtattgctgctttgttacaaaatgttctaaagtggaaacactgtatatagaccaggagcagtggctcaggcatgtaatcccagtaccttgggaggccaaggtgggtagatcccttgagctcaggagtttgagaccagcctagacaacattgtgaaaccccacctctacgcaatctaaaaattcagccggagaatgcagcgtacacctgtagggccagctattatgggactgaggctggaggatcaattgagcccaagaggtggaggcagtagcaactgtgtttgtgccactgcactccaacctgggtgacaaagtgagaccctgtatcaagaaaaaaaaactaaaacacagtatccaaaagtcaaactgggccagacgcagtggctcacacctgtaatcccagcactttgggaggccaaggcgggcagatcacctgaggtcaggagtttcagaccagcctggccaacatggtgaaaccccgtctctattaaaacaaaaagccaggcgtggtgcacgtacctgtagtcccatttactcaggaggctgaggcaggagaatcacttgaacatgggaggcagaggttgcagtgagcaaagatcccaccactgcactccagcctgggtgacagagcgagtctgtgtctcacaaaaaagtgtcaagctaagagttgctttcccttcccactgaaacagttgaagcgctgaaaacaggctaggggcagccctgccagtccaggagaactttctgcaatgactagaacgttctatataccgtttacttaaacatcttacaaattcaataaaattagaatcagttcctcagtcctgccacacttcagttgcttagcagccacacatgtatgtggccagtggcaactgttggacaagtcaagtctaga

SEQ ID NO: 4 - DNA, 28 bp, Homo sapiens
cgcggatccagaagattccaggttcaag

SEQ ID NO: 5 - DNA, 29 bp, Homo sapiens
gctggatccagttctatacagatttacat

SEQ ID NO: 6 - DNA, 40 bp, Homo sapiens
gactgcgcgcagaaggagagagatgaccccgccttccgag

SEQ ID NO: 7 - DNA, 29 bp, Homo sapiens
gcaggctagcttaggcctcccgcagcttc

SEQ ID NO: 8 - DNA, 18 bp, Homo sapiens
ctaatcacaacacttagc

SEQ ID NO: 9 - DNA, 18 bp, Homo sapiens
actagaggaggccaagag